(19) United States
(12) Patent Application Publication
(10) Pub. No.: US 2025/0075249 A1
(43) Pub. Date: Mar. 6, 2025

(54) AND-GATE ALLOSTERIC PROTEIN-BASED SWITCHES

(71) Applicant: QUEENSLAND UNIVERSITY OF TECHNOLOGY, Brisbane (AU)

(72) Inventors: Kirill ALEXANDROV, Brisbane (AU); Zhong GUO, Brisbane (AU)

(21) Appl. No.: 18/724,023

(22) PCT Filed: Jan. 4, 2023
(86) PCT No.: PCT/IB2023/020001

§ 371 Date: Jun. 25, 2024

(30) Foreign Application Priority Data
Jan. 5, 2022 (GB) 2200051.7

(51) Int. Cl. C12Q 1/26; C07K 14/47; C12N 9/04; C12N 9/24; C12N 9/86; C12Q 1/32; C12Q 1/34

(52) U.S. Cl. CPC C12Q 1/26; C07K 14/4728; C12N 9/0006; C12N 9/2402; C12N 9/86; C12Q 1/32; C12Q 1/34; C12Y 101/01047; C12Y 302/01028; C12Y 305/02006

(57) ABSTRACT

The present invention relates to improved protein-based biosensors that are suitable for detection of one or more target molecules in a sample. The biosensors are fully-reversible with dynamic ranges suitable for analytic and diagnostic applications. The biosensors of the present invention may be used in synthetic biology, for example in constructing artificial cellular or extracellular signalling networks.

### FIELD OF THE INVENTION

The present invention relates to improved biosensors. In particular, the present invention relates to improved protein-based biosensors that are suitable for detection of one or more target molecules in a sample. The biosensors of the present invention may also relate to the field of synthetic biology such as for constructing artificial cellular or extracellular signalling networks. The biosensors are fully-reversible with dynamic ranges suitable for analytic and diagnostic applications.

### BACKGROUND

Construction of artificial protein switches with selectable input and output parameters is a key goal of synthetic biology and biotechnology. Protein biosensors have application in clinical and industrial analytics, particularly in point-of-need diagnostics. The direct conversion of analyte binding into a quantitative signal, in a simple, wash-free, homogeneous assay format is an un-met need in this field. Furthermore, artificial protein-based switches that interconvert different biochemical signals also enable construction of biochemical pathways with non-native proteins for metabolic, cellular and organismal engineering.

There are several competing approaches to the construction of protein biosensors, all of which aim to create an OFF state of the desired reporter domain that can be reversed by the analyte of choice. Previously described approaches to constructing protein-based biosensors include domain splitting, creation of reversible auto-inhibition, and insertion of regulatory domains. Regulatory domain insertion provides direct reversible coupling between ligand binding and reporter activity, but is reliant on small ligand-binding domains with global conformational changes that are sufficiently large to modulate the activity of the reporter. Previously described biosensors addressed this problem by using peptide-receptor domains with large conformational changes and then applying protein engineering to couple local concentration of the peptide ligand to that of the analyte. Such protein-based biosensors are described for example in WO 2016/191812, WO 2018/073588 and WO 2019/207356.

The key parameters determining the utility of the biosensor are selectivity, sensitivity, dynamic range and the rate of response. Dynamic range is particularly important as it determines the signal-to-noise ratio and the accuracy with which measurement of the target analyte can be conducted. Construction of proteins switches with practically relevant dynamic ranges is far from trivial and often requires extensive optimisation of constructs generated through screening. Given the ensemble nature of allostericity and an equilibrium nature of ligand binding it is typically expected that there will be trade-off between the dynamic range and reversibility of the system. Thus, systems that disfavour one of the states, (such as, e.g., split proteins), are expected to demonstrate large dynamic ranges but poor/slow reversibility. For example, in one exemplary system, a cysteine bond was introduced into the linker between a calmodulin regulatory domain insert and a glucose dehydrogenase reporter domain. Formation of a disulphate bond upon structural rearrangement of the receptor domain increased the dynamic range from 4 fold to nearly 100 fold, but at the expense of reversibility.

The present application addresses these problems. Described herein is an approach for construction of fully-reversible protein switches with exceptionally large dynamic ranges and fast activation times. This is achieved by constructing integrated protein AND gates.

### SUMMARY OF THE INVENTION

The present invention relates to improved reporter proteins, such as enzymes, which may be used in biosensors. In particular, the improved enzymes are particularly adapted for detection of target molecules in physiological conditions, for example in biological samples. The improved enzymes comprise multiple heterologous amino acid sensor domains, are fully reversible and have improved sensitivity, enhanced dynamic ranges and fast response times, making them specifically adapted for detection of target molecules that are typically at low concentrations in physiological conditions and allowing for simultaneous detection of multiple target molecules.

Thus, the present invention provides a reporter protein comprising a first heterologous amino acid sequence which is responsive to binding of a first regulator moiety and a second heterologous amino acid sequence which is responsive to binding of a second regulator moiety, wherein binding of the first regulator moiety to the first heterologous amino acid sequence and binding of the second regulator moiety to the second heterologous amino acid sequence, reversibly regulates the activity of the reporter protein.

Also provided are biosensors comprising a reporter protein as described herein and one or more (e.g., at least one) regulator moiety as described herein (such as a first regulator moiety as described herein and a second regulator moiety as described herein). Also provided are as described herein. Also provided herein is a method of detecting one or more target molecules, comprising contacting a reporter protein, or a biosensor, or a composition, or a kit, as described herein; with a sample under conditions suitable for detection of the presence or absence of the one or more target molecules in the sample.

The present invention further provides: (i) a method of diagnosis of a disease or condition in an organism, comprising contacting a reporter protein, a biosensor, a composition, or a kit, as described herein, with a sample obtained from the organism under conditions suitable for detection of the presence or absence of the one or more target molecules in the sample, wherein presence or absence of the one or more target molecules in the sample is indicative of whether the organism has, or is at risk of having, said disease or condition; (ii) a method of monitoring one or more target molecules in an organism, the method comprising contacting a reporter protein, a biosensor, a composition, or a kit, as described herein,, with a sample obtained from the organism under conditions suitable for detecting and/or quantifying the presence or absence of the one or more target molecules in the sample; (iii) a method of monitoring a metabolite of interest in an organism, the method comprising expressing a reporter protein, or a biosensor, as described herein, in said organism under conditions suitable for detecting and/or quantifying the metabolite of interest in the organism, optionally wherein the organism is a bacterial cell; or (iv) a method of assaying for protein-protein or protein-small molecule interactions comprising contacting a reporter protein, a biosensor, a composition, or a kit, as described herein, with a sample under conditions suitable for detection of the presence or absence of an interaction between binding moieties B1′ and B1″ and/or an interaction between binding moieties B2′ and B2″; or an interaction between binding moieties B1′ and B1″ and a target molecule TM1 and/or an interaction between binding moieties B2′ and B2″ and a target molecule TM2.

Also provided is a detection device that comprises a cell or chamber that comprises a reporter protein or a biosensor, as described herein. Also provided is one or more nucleic acids encoding a reporter protein or a biosensor, as described herein. Also provided is one or more expression vectors comprising said one or more nucleic acids operably linked to one or more promoters. Also provided is a host cell comprising a reporter protein, a biosensor, or one or more nucleic acids or one or more expression vectors, as described herein. Also provided is a method for expressing a reporter protein or a biosensor, as described herein, in a host cell, the method comprising incubating a host cell, as described herein, under suitable conditions for expression of the reporter protein or biosensor.

Further provided is a method for converting a constitutively active enzyme into a reversibly regulated enzyme whose catalytic activity is dependent on the presence of one or more target molecules, the method comprising: (a) generating a library of single insert enzyme mutants by inserting a heterologous amino acid sequence which is responsive to binding of a regulator moiety at a number of different locations in an enzyme sequence and selecting those single insert enzyme mutants showing a change in catalytic activity on binding of the regulator moiety; and (b) generating a library of double insert enzyme mutants by inserting an additional heterologous amino acid sequence which is responsive to binding of a regulator moiety at a second site within the enzyme sequence, and selecting those double insert enzyme mutants showing a change in catalytic activity in the presence of the regulator moiety and having an increased dynamic range as compared to the corresponding single insert enzyme mutants; optionally wherein the change in catalytic activity occurs within less than 10 minutes, optionally less than 5 minutes.

### DESCRIPTION OF THE FIGURES

FIG. 1. Design of artificial allosteric enzymes by domain insertion. (A) A schematic representation of a constitutively active reporter protein domain. (B) Construction of a chimeric protein by insertion of a receptor domain. exemplified by calmodulin, into the reporter protein. (C) Activation of the reporter protein's activity through interaction of the chimera with a ligand that induces conformational change in the receptor domain (exemplified by calmodulin binding peptide). (D) A schematic representation of an enzymatic cascade for conversion of trehalose to gluconolactone by trehalase (Tre) and PQQ-Glucose Dehydrogenase (PQQ-GDH).

FIG. 2. Construction of and activity analysis of Trehalase-CaM chimeras. (A) A schematic representation of chimeric Tre construction. (B) A schematic representation of Tre-CaM activation by Calmodulin binding peptide (CaM-BP). (C) Structure of trehalase (PDB: 2jg0) displayed in ribbon representation with insertion positions shown as balls. (D) Activity analysis of a solution of 50 nM Tre-CaM mutants mixed with 50 nM GDH in the presence of 50 mM Trehalose. 0.6 mM PMS as electron mediator and 60 μM DCPIP reporter dye in the presence (interrupted trace) or absence (solid trace) of 0.5 μM of M13 CaM-BP. The identity of mutants is indicated by their insertion position.

FIG. 3. Construction of Tre chimeras with two CaM regulatory domains. (A) A schematic representation of the design process based on a combination of the single insert variants identified using the screening approach outlined in FIG. 2D. (B) Activity analysis of a 50 nM solution of Tre G104_S440 CaM chimera in the presence or absence of 0.5 μM M13 CaM-BP. The assay was performed as in FIG. 2D. (C) As in (B), but using TreG104_P321 CaM chimera. (D) Activity of 50 nM solution of Tre G104_P321 chimera in the presence of different concentrations of M13 CaM-BP peptide. (E) Fit of the data from titration shown in (D) to an apparent K_{d} value of 65 nM. (F) A ribbon representation model of Tre G104_P321 CaM chimera. Calcium ions are displayed as balls while the M13 peptides are displayed as ribbons.

FIG. 4. Using 2CaM-Tre switch module to construct small molecule AND gate biosensors with large dynamic ranges. (A) Schematic representation of a rapamycin biosensor based on an AND gate switch, in which a 2CaM-Tre switch module is tagged on the C and N terminus with FKBP domains. Activating domain is composed of FRB fusion with a low affinity CaM-BP peptide. Addition of the rapamycin drives association of the components and activation of the trehalase activity. (B) Titration of 100 nM 2FKBP-Tre-2CaM and 200 nM FRB-CaM-BP with increasing concentrations of rapamycin. (C) A fit of the titration data shown in (B) to an apparent K_{d} of 89 nM. (D) Construction of a biosensor based on an AND gate Tre switch modulated by two inputs. Here a Tre-2CaM switch module is tagged with FKBP on the N-terminus and with Cyclophilin domain on the C-terminus. FRB-CaM-BP and a fusion of calcineurin A and B (CalA/B) with CaM-BP are used as the activator. (E) Activity of two input system shown in (D) in the presence of Rapamycin, Cyclosporine A or their mixture. In the experiment 100 nM solution of FKBP-Tre-2CaM-Cyclophilin were mixed with 100 nM FRB-CaM-BP and 100 nM CalA/B-CaM-BP and 50 nM wt GDH in the presence or absence of 350 nM of Rapamycin, Cyclosporine or their mixture, and changes in absorbance of DCIP dye were recorded over time. (F) In order to endow the system shown in D with an additional level of regulation the AND gate biosensor shown in (A) was combined with a two component GDH biosensor of Cyclosporine A. (G) A Cyclophilin (CyP) domain fused CaM-GDH chimera is activated by CalA/B fused with CaM-BP in the presence of by Cyclosporine A. (H) Time trace of GDH activity of the system shown in (F) and (G), where 100 nM FKBP-Tre-2CaM-FKBP were mixed with 200 nM of FRB-CaM-BP, 5 nM CaM-GDH-Cyclophilin domain and 30 nM CalA/B-CaM-BP in the presence or absence of 250 nM Rapamycin, Cyclosporine A or their mixture.

FIG. 5. Construction of β-lactamase single insert CaM chimeras (BLA-CaM). (A) A schematic showing th β-lactamase (BLA) assay. BLA degrades the beta-lactam ring nitrocefin. which can be detected by a colour change from yellow to red, for example using a wavelength λ=482 nm (wavelengths of λ=486 nm and λ=480 nm may also be used). (β) Structure of β-lactamase in ribbon representation (PDB: 3gmw) with calmodulin insertion position displayed as balls. (C) Activity of purified BLA-CaM chimeras in the presence (interrupted line) or absence (solid line) of saturating concentrations of M13 peptide. The insertion position is indicated by a number and a letter denoting deleted amino acid.

FIG. 6. Construction of B-lactamase double insert CaM chimeras (2CaM-BLA). (A) Activity analysis of a solution of 250 nM BLA-CaM 41G in the presence of 50 μM nitrocefin and in the presence (interrupted line) or absence (solid line) of 1 μM of M13 CaM-BP. (B) Activity of the reaction mixture from (A), but with 25 nM BLA-CaM 41G pre-incubated for the indicated periods of time before addition of nitrocefin. (C) Activity analysis of a solution of 250 nM BLA-CaM 197E in the presence of 50 μM nitrocefin and in the presence (interrupted line) or absence (solid line) of 1 μM of M13 CaM-BP. (D) Activity of the reaction mixture from (C) but with 25 nM BLA-CaM 197E pre-incubated for the indicated periods of time before addition of nitrocefin. (E) A schematic representation of chimeric BLA biosensor containing two CaM modules. (F) Activity of 250 nM BLA-CaM 41G_197E in the presence of 50 μM nitrocefin in the presence (interrupted line) or absence (solid line) of 1 μM of M13 CaM-BP. (G) Activity of the reaction mixture from (F), but with 10 nM BLA-CaM 41G_197E pre-incubated for the indicated periods of time before addition of nitrocefin. (H) A plot of activity of 10 nM of wild type β-lactamase, single CaM insertion β-lactamase and double CaM CaM41G and CaM197E insertion β-lactamase in the presence of saturating concentrations of M13 CaM-BP. (I) A plot of maximal activity of 10 nM of wild type β-lactamase, double CaM CaM41G and CaM197E insertion β-lactamase and single insertion CaM-BLA 253G chimera with large dynamic range of 207 fold.

FIG. 7. Rapamycin biosensor based on 2CaM-BLA switch. (A) A schematic representation of a two component biosensor of rapamycin based on a BLA-CaM 41G_197E switch unit. (B) Activity analysis of a solution of 25 nM BLA-2CaM-2FKBP fusion protein, and 100 nM FRB-CaM-BP (low affinity calmodulin binding peptide), in the presence of 50 μM nitrocefin, and increasing concentrations of rapamycin. (C) Fit of the data shown in (B) to a quadratic equation leading to an apparent K_{d} value of 11 nM. (D) Activity analysis of a solution of 25 nM BLA-2CaM-2FKBP fusion protein. 100 nM FRB-CaM-BP and 250 nM of rapamycin incubated before addition of 50 μM nitrocefin for the indicated periods of time.

FIG. 8. Tacrolimus biosensor based on 2CaM-BLA switch. (A) A schematic representation of a two component biosensor of Tacrolimus, based on BLA-CaM 41G_197E switch unit. (B) Activity analysis of a solution of 25 nM BLA-2CaM-2FKBP fusion protein and 100 nM CalcineurinA/B fusion-CaM-BP (low affinity calmodulin binding peptide), in the presence of 50 μM nitrocefin, and increasing concentrations of Tacrolimus. (C) Fit of the data shown in (B) to a quadratic equation leading to an apparent K_{d} value of 14 nM. (D) Activity analysis of a solution of 25 nM BLA-2CaM-2FKBP fusion protein. 100 nM FRB-CaM-BP and 250 nM of tacrolimus incubated before addition of 50 μM nitrocefin for the indicated periods of time.

FIG. 9. Methotrexate biosensor based on 2CaM-BLA switch. (A) A schematic representation of a two component biosensor of methotrexate (MTX), based on BLA-CaM 41G_197E switch unit. (B) A time trace of absorption changes of the following solutions, as indicated by arrows: 10 nM BLA-2CaM 41G197. 10 nM BLA-2CaM 41G197 and 1 μM M13 CaM-BP added at 10 minute mark. 10 nM VHH-BLA-2CaM 41G197-VHH mixed with 100 nM nanoCLAMP-BP. 10 nM VHH-BLA-2CaM 41G197-VHH mixed with 100 nM nanoCLAMP-BP and 250 nM methotrexate added at 10 minute mark. (C) Activity of 10 nM VHH-BLA-2CaM 41G197-VHH and 100 nM nanoCLAMP-BP solution supplemented with indicated concentrations of methotrexate. (D) Fit of the data shown in (C) to a quadratic equation leading to an apparent K_{d} value of 7.3 nM. (E) Activity analysis of a solution of 10 nM VHH-BLA-2CaM 41G197-VHH and 100 nM nanoCLAMP-BP. 250 nM MTX incubated before addition of 50 μM nitrocefin for the indicated periods of time. (F) Plot of the data shown in (E) fitted to a single exponential.

FIG. 10. Use of the developed 2CaM-BLA biosensor for construction of a clinically useful MTX assay. (A) A picture of a Beckman AU-480 clinical chemistry analyser used to test two component MTX biosensor based on β-lactamase double CaM CaM41G and CaM197E switch module. (B) Time trace of 20 mM Tris pH7.4. 100 mM NaCl buffer containing 10 nM VHH-BLA-2CaM 41G197-VHH and 100 nM nanoCLAMP-BP and 50 μM nitrocefin supplemented with indicated concentrations of methotrexate standards in human serum (Abbot Architect MTX calibration kit). (C) Plot of data shown in (B) fitted to a linear function. (D) Analysis of serum samples of 21 patients undergoing MTX therapy using 2CaM-BLA MTX biosensor (X-axis) or Abbot Architect immunochemistry station (Y-axis).

FIG. 11. Construction of a glucose dehydrogenase (GDH) AND gate biosensor. (A) GDH-CaM 48N activation by CaM-BP. activity analysis of a solution of 10 nM GDH-CaM 48N in the presence of 0.6 mM PMS as electron mediator and 60 μM DCPIP reporter dye in the presence of absence of 1 μM of M13 CaM-BP. (B) GDH-CaM 212N activation by CaM-BP. activity analysis of a solution of 10 nM GDH-CaM 212N in the presence of 0.6 mM PMS as electron mediator and 60 μM DCPIP reporter dye in the presence of absence of 1 μM of M13 CaM-BP. (C) GDH-CaM 48N_212N activation by CaM-BP. activity analysis of a solution of 10 nM GDH-CaM 48N_212N in the presence of 0.6 mM PMS as electron mediator and 60 μM DCPIP reporter dye in the presence of absence of 1 μM of M13 CaM-BP.

FIG. 12. Schematic representation of a biosensor design that requires only one ligand binding domain. (A) A biosensor where Binder 2 is operably linked to two CaM-BPs cach having low affinity for calmodulin. (B) A biosensor design where ligand binder 2 is operably linked between two fusion proteins, wherein cach fusion protein comprises a wild type CaM-BP and a calmodulin domain having low affinity for said CaM-BP. Addition of ligand (star) leads to an interaction between ligand binder 1 and ligand binder 2 with the ligand. Co-localisation of the ligand binder 2 fusion protein with the reporter domain (black square) leads to relocation of the CaM-BPs to the high affinity calmodulin modules on the reporter domain, thereby leading to activation of the biosensor.

FIG. 13. Use of 2CaM-reporter units to monitor activity of proteases. (A) A schematic showing a fusion protein wherein a CaM-BP is caged in a calmodulin mutant having low affinity for Cam-BP. The dashed line represents a protease cleavage site. (B) As in (A) but with unit duplication that leads to higher avidity of the component and reduces background activity. (C) A caged unit with duplicated cage designed to decrease background activity of the caged peptide. (D) As in (C) but with a protease binding domain designed to increase the local concentration of the protease, thereby increasing system's specificity for a particular protease and increasing the rate of the proteolysis.

FIG. 14. Electrochemical detection of β-lactamase activity. Cyclic voltammograms of 0.5 mM Nitrocefin (dashed line) and products of its enzymatically hydrolysis (solid line). Conditions: carbon paper-based electrode (0.5×0.5 cm dipped); scan rate 50 mV/s vs. Ag/AgCl (3M KCl); 100 mM PBS. pH 7.2; β-lactamase (150-300 U/mL); time of enzymatic reaction 10 min; all experiments were performed in dark at room temperature.

FIG. 15. Expression of BLA-CaM fusion proteins in E.coli and analysis of the ability of transgenic bacteria to survive on ampicillin. (A) A plasmid map of modified pACYDuet vector designed to constitutively express proteins of interest in E.coli cells. (B) Optical density of DH5-alpha E. coli strain transformed with pACYDuet-BLA-CaM253G grown overnight in LB medium containing Chloramphenicol at concentration of 34 μg/ml and ampicillin at concentration of 100 μg/ml in the presence of the indicated concentration of CaM-BP. (C) An agar plate containing 100 μg/ml ampicillin and 34 μg/ml chloramphenicol seeded with cells transformed with pACYDuet-BLA-CaM253G vector. After plating, the indicated concentrations of CaM-BP were spotted onto the indicated positions of the plate, and the plate was incubated at 37° C. overnight. Bacterial growth appears as dark spots.

FIG. 16. Using CaM-BP to control ampicillin resistance of bacteria transformed with BLA-CaM fusion. Agar plates containing 34 μg/ml chloramphenicol (left) or 100 μg/ml ampicillin and 34 μg/ml chloramphenicol (right), and seeded with DH5-alpha cells transformed with the indicated constructs and grown at 37° C. overnight.

FIG. 17. Constitutive expression of BLA-2CaM fusion proteins in E.coli and analysis of the ability of transgenic bacteria to survive on ampicillin. (A) A plasmid map of modified pACYDuet-modified-BLA-2CaM 41G 197E vector designed to express proteins of interest in DH5-alpha E. coli strain. (B, C) Agar plates containing 34 μg/ml chloramphenicol (B) or 34 μg/ml chloramphenicol and 100 μg/ml ampicillin (C) and seeded with cells transformed PACYDuet-modified-BLA-2CaM 41G 197E vector. (D) Optical density of DH5alpha cells transformed with pACYDuct-modified-BLA-2CaM 41G 197E vector grown overnight in LB medium containing Chloramphenicol at a concentration of 34 μg/ml and Ampicillin at a concentration of 100 μg/ml in the presence of indicated concentration of CaM-BP. (E) An agar plate containing 100 μg/ml ampicillin and 34 μg/ml chloramphenicol seeded with cells transformed PACYDuet-modified-BLA-2CaM 41G 197E vector. After plating indicated concentrations of CaM-BP were spotted onto the indicated positions of the plate and the plate was incubated at 37° C. overnight. Bacterial growth appears as dark spots.

FIG. 18. Inducible expression of BLA-2CaM fusion proteins in E.coli and analysis of the ability of transgenic bacteria to survive on ampicillin. (A) A plasmid map of modified PET28a-BLA-2CaM 41G 197E vector designed to inducibly express proteins of interest in BL21 (DE3) E.coli cells. (B) Optical density of BL21 (DE3) E.coli cells transformed with PET28a-BLA-2CaM 41G 197E grown overnight in LB medium containing kanamycin at a concentration of 50 μg/ml and ampicillin at a concentration of 100 μg/ml in the presence of indicated concentration of CaM-BP and without and with 1 mM of IPTG. (C) SDS-PAGE gel loaded with the cells from experiment shown in (B) the lane 1 is loaded with purified BLA-2CaM 41G 197E protein. An agar plate containing 100 μg/ml ampicillin and 50 μg/ml kanamycin seeded with cells transformed with the PET28a-BLA-2CaM 41G 197E vector. After plating. the indicated concentrations of CaM-BP and 1 mM IPTG were spotted onto the indicated positions of the plate and plate was incubated at 37° C. overnight. Bacterial growth appears as dark spots.

FIG. 19. Emission scan of mNeon chimera fluorescent protein with one or two calmodulin domains. A 1 μM solution of mNeon-CaM chimera (small grey diamonds) and mNcon-2CaM (black balls) were scanned for emission, keeping excitation wavelength at 500 nm±5 nm constant. The scan was repeated upon addition of 5 μM of M13 calmodulin binding peptide mNeon-CaM chimera (grey triangles) and mNeon-2CaM (black diamonds).

FIG. 20. Activity analysis of GDH affinity clamp single and double insert chimeras. (A) Activity analysis of a solution of 10 nM GDH-affinity clamp chimera where the affinity clamp was inserted at the position 46. The assay was performed in the absence (solid line) and in the presence (dashed line) of 1 μM of RGS ligand peptide. The assay was performed as described in the description for FIG. 11. (B) As in (A) but using a solution of GDH-affinity clamp chimera where the affinity clamp was inserted at the position 54. (C) As in (A) and (B), but using a solution of GDH-affinity clamp chimera where two affinity clamps were inserted at the positions 46 and 54.

FIG. 21. Using biosensors based on 2CaM-Tre and 2CaM-GDH switch modules to construct biosensor systems with large dynamic ranges regulated by two different inputs. (A) Schematic representation of the allosteric enzymatic cascade regulated at two different nodes. Here AND gate Tre A-based biosensor controlled by rapamycin is combined with AND gate GDH biosensor of Methotrexate (B) Activity plot of the enzymatic cascade shown in A where 100 nM FKBP-2CaM-Tre-FKBP were mixed with 200 nM of FRB-CaM-BP. 20 nM 2CaM-GDH-2VHH domain and 100 nM nanoCLAMP-CaM-BP in the presence or absence of 250 nM rapamycin. methotrexate or their mixture.

FIG. 22. Thermostabilised Methotrexate biosensor based on 2CaM-BLA switch (A) Activity of 10 nM solution of 2CaM-BLA-41G 197E in the presence of saturating concentrations of M13 CaM-BP after incubation at indicated temperatures for the following periods of time: 4° C.-3 hours. 25° C.-3 hours. 37° C.-1 hour. 50° C.-30 minutes. 60° C.-10 minutes and 80° C.-10 minutes. (B) as in A but using K55Q. S82A. G92D. T140K. H153R. V184A mutant of 2CaM-BLA-41G 197E. (€) Change of absorbance of 10 nM of 2CaM-BLA-41G/197E and its K55Q. S82A. G92D. T140K. H153R. V184A mutant in the absence and presence of 1 μM CaM-BP. (D) Relative activity of 10 nM of 2CaM-BLA-41G 197E and its K55Q. S82A. G92D. T140K. H153R, V184A mutant in the presence of saturating concentrations of M13 peptide. (E) The activity of 10 nM solution of methotrexate biosensor based on 2VHH 2CaM-BLA-41G 197E K55Q, S82A, G92D, T140K, H153R, V184A mutant mixed with 100 nM of nanoCLAMP-CaM-BP titrated with increasing concentrations of methotrexate. (F) A plot of Kobs rates from C fitted to a quadratic equation leading to a K_{d} value of 8.6±1.8 nM.

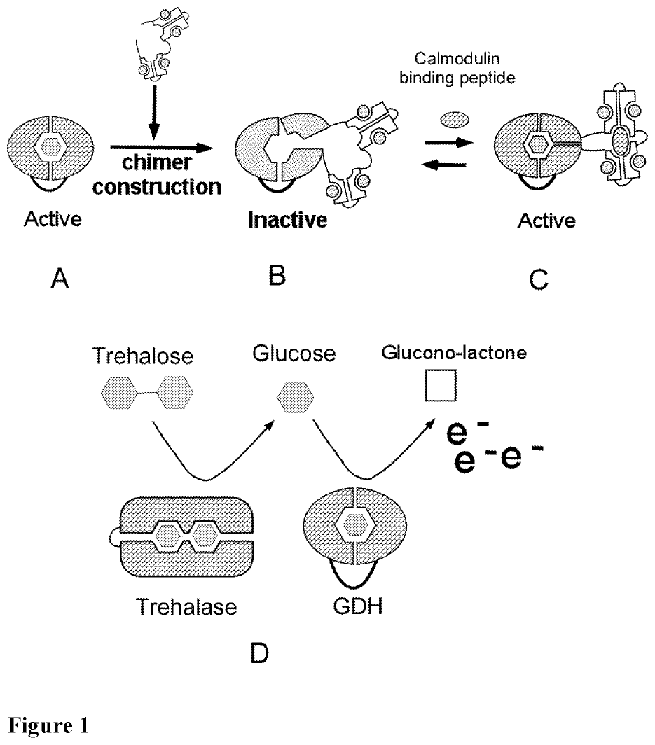

### DETAILED DESCRIPTION

The present invention relates to improved reporter proteins, which may be used in biosensors that are preferably capable of detecting a target molecule. Typically, the reporter proteins comprise two or more (i.e., more than one, a plurality of) heterologous amino acid sequences, which act as sensor domains and undergo a conformational change upon binding of a regulator moiety. The two or more heterologous amino acid sequences may be the same or different sequences. Binding of regulatory moieties to the two or more heterologous amino acid sequences induce conformational changes in the two or more heterologous amino acid sequences, which epistatically interact (i.e., have an additive effect) and confer a large-scale conformational change on the reporter protein to switch it from an “OFF” state to an “ON” state, or from an “ON” state to an “OFF” state. The reporter protein then produces a detectable signal which may be detected and/or quantified. By coupling the association of each of the heterologous amino acid sequences with their respective regulator moieties to be dependent on the presence of one or more target molecule, for example by linking the heterologous amino acid sequences and regulator moieties to a pair of binding moieties configured to bind a target molecule, then activation of the reporter protein may be dependent on the presence of one or more target molecules. The reporter proteins and biosensors described herein have the advantage of large dynamic ranges within clinically useful response times and also allow for multiple different target molecule inputs.

Thus, the present invention provides a reporter protein (such as an enzyme, a fluorescent protein or a binding domain, preferably an enzyme or a fluorescent protein, most preferably an enzyme) comprising a first heterologous amino acid sequence which is responsive to binding of a first regulator moiety and a second heterologous amino acid sequence which is responsive to binding of a second regulator moiety, wherein binding of the first regulator moiety to the first heterologous amino acid sequence and binding of the second regulator moiety to the second heterologous amino acid sequence, reversibly regulates (preferably reversibly activates) the activity of the reporter protein (such as the catalytic activity of the enzyme, or the fluorescence of the fluorescent protein). Typically, each heterologous amino acid sequence (i.e., the first and the second heterologous amino acid sequences) is provided as an insert within the amino acid sequence of the reporter protein (i.e., enzyme or fluorescent protein).

One advantage of the claimed reporter proteins and the biosensors comprising them, is a remarkably improved dynamic range, whilst still maintaining reversible regulation of the activity of the reporter protein. The dynamic range of the reporter protein (which is preferably an enzyme) may be at least 5 fold, 10 fold, 20 fold, 30 fold, 40 fold, 50 fold, 75 fold, 100 fold, or 150 fold. The dynamic range of the reporter protein is optionally at least 10 fold, preferably at least 20 fold. The dynamic range of a biosensor comprising the reporter protein as described herein may be at least 5 fold, 10 fold, 20 fold, 30 fold, 40 fold, 50 fold, 75 fold, 100 fold, or 150 fold. The dynamic range of a biosensor comprising the reporter protein as described herein is optionally at least 10 fold, preferably at least 20 fold. The dynamic range provides a measure of the ratio between the maximal achievable signal and the background activity of the biosensor/reporter protein under optimal conditions for detection. The larger the dynamic range the better the signal-to-noise ratio of the biosensor/reporter protein, i.e., the better the sensitivity of the biosensor/reporter protein, and the smaller the changes in the concentration of the target molecule that may be detected by the biosensor/reporter protein. The dynamic range of the biosensors/reporter proteins of the invention may be calculated by comparing the activity of the biosensor/reporter protein in the absence of target molecules to the activity of the biosensor/reporter protein when target molecules are present in saturating concentrations, i.e., a concentration of target molecules where further increases in concentration do not increase the activity of the biosensor/reporter protein any further. Thus, the dynamic range (X) may be defined as the maximum possible signal level of the biosensor/reporter protein (i.e., in the presence of saturating concentrations of target molecules, and under optimal reaction and detection conditions) (B), divided by the background activity level, which is the signal level of the biosensor/reporter protein when there is no target molecule present in the assay (A).

Thus, the dynamic range X =B/A. For example, if the maximum possible signal level, B, is 60, and the background activity level, A, is 30, the dynamic range, X=B/A=60/30=2, i.e., a dynamic range of 2 fold. For example, a dynamic range of 10 fold may be provided by a biosensor having background activity level of 1 and a maximum signal level of 10 (X=B/A=10/1=10fold). The signal level may be defined as the observed reaction rate (i.e., the initial reaction velocity) obtained by fitting the linear phase of the reaction curve.

### Definitions

The term “variant” as used herein (for example as used in connection with the reporter proteins, heterologous amino acid sequences, or regulator moieties described herein) may describe functional fragments of amino acid sequences, proteins or peptides of the invention, suitably retaining their relevant activity or function, catalytic activity or binding activity as applicable. Variants may include amino acid sequences comprising deletion or insertions as compared to any of the amino acid sequences disclosed herein. Such deletions or insertions may be 1, 2, 3, 4, 5, 6, 7, 8, 9 or 10 amino acids in length, or between 1-5, 1-10, 1-20, 1-30, 1-40 or 1-50 amino acids in length, preferably between 1-10 amino acids in length. Optionally such deletions or insertions occur in loops or at the N- or C-terminus. Variants may preferably include amino acid sequences comprising mutations (i.e., substitutions, point mutations) relative to the corresponding wild type amino acid sequence or relative to the corresponding amino acid sequence disclosed herein. Variants may comprise up to 1, 2, 3, 4, 5, 6, 7, 8, 9, 10, 15, 20, 25, 30, 40, 50, 60, 70, 80, 90 or 100mutations, or between 1-5, 1-10, 1-15, 1-20, 1-30, 1-40 or 1-50 mutations, optionally between 1-20, preferably between 1-10, most preferably between 1-5, mutations relative to the corresponding wild type amino acid sequence or relative to the corresponding reference amino acid sequence disclosed herein. For example, typically, conservative amino acid variations may be made without an appreciable or substantial change in function. For example, conservative amino acid substitutions may be tolerated where charge, hydrophilicity, hydrophobicity, side chain “bulk”, secondary and/or tertiary structure (e.g. helicity), regulator moiety binding, target molecule binding, fluorescence, enzyme activity and/or inhibitory activity are substantially unaltered or are altered to a degree that does not appreciably or substantially compromise the function of the biosensor. Variants may include amino acid sequences having at least 80%, 85%, 90%, 91%, 92%, 93%, 94% 95%, 96%. 97%, 98% or 99%, preferably 80%, more preferably 90%, most preferably 95%, sequence identity with any of the amino acid sequences disclosed herein.

The term “fragment” as used herein (for example as used in connection with the reporter proteins, heterologous amino acid sequences, or regulator moieties described herein) typically describes functional fragments of amino acid sequences, proteins or peptides of the invention, suitably retaining their relevant activity or function, catalytic activity or binding activity as applicable. Fragments are typically N- and/or C-terminal truncations. Protein fragments may comprise up to 5%, 10%, 15%, 20%, 25%, 30%, 35%, 40%, 45%, 50%, 55%, 60%, 65%, 70%, 75%, preferably up to 80%, 85%, more preferably up to 90% or most preferably from 95% to 99% of an amino acid sequence disclosed herein. In some embodiments, the protein fragment may comprise up to 5, 10, 20, 40, 50, 70, 80, 90, 100, 120, 150, 180 200, 220, 230. 250, 280, 300, 330, 350, 400 or 450 amino acids of an amino acid sequence disclosed herein. In some embodiments, the protein fragment may include a deletion of 1-10, 1-20, 1-30, 1-40, 1-50, 1-75, 1-100, preferably 1-50, most preferably 1-100 amino acids relative to the wild type sequence of the protein, typically the deletion occurs at the N- and/or C-terminus. The fragment is typically a functional fragment, in that it retains the activity of interest (i.e., the activity relevant to the biosensor as described herein) of the corresponding wild-type protein. The fragment may comprise a domain comprised within a full-length protein, typically the protein domain comprised within the fragment is the protein domain responsible for the activity of interests in the wild-type protein. The fragment may comprise up to 5, 10, 20, 40, 50, 70, 80, 90, 100, 120, 150, 180 200, 220, 230. 250, 280, 300, 330, 350, 400 or 450 amino acids of an amino acid sequence disclosed herein.

“Sequence identity”, as used herein, may be determined by sequence comparison and alignment algorithms known in the art. To determine the percent identity of two protein/polypeptide/ peptide or two nucleic acid/polynucleotide sequences, the sequences are aligned for optimal comparison purposes (e.g., gaps can be introduced in the first sequence or second sequence for optimal alignment). The amino acid residues or nucleotide residues at corresponding positions are then compared. When a position in the first sequence is occupied by the same residue as the corresponding position in the second sequence, then the molecules are identical at that position. The percent identity between the two sequences is a function of the number of identical positions shared by the sequences (i.e., % homology= #of identical positions/total #of positions*100), optionally penalizing the score for the number of gaps introduced and/or length of gaps introduced. Sequence identity is typically measured over the length of the shorter sequence.

The comparison of sequences and determination of percent identity between two sequences can be accomplished using a algorithm. The alignment may be generated over a certain portion of the sequence aligned having sufficient identity but not over portions having low degree of identity (i.e., a local alignment). A non-limiting example of a local alignment algorithm utilised for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

The alignment may optimised by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. The alignment may be optimised by introducing appropriate gaps and percent identity may be determined over the entire length of the sequences aligned (i.e., a global alignment). A non-limiting example of an algorithm utilized for the global comparison of sequences is the algorithm of Myers and Miller, CABIOS (1989). Such an algorithm is incorporated into the ALIGN program (version 2.0) which is part of the GCG sequence alignment software package.

Sequence identity between two sequences is preferably determined using pairwise global sequence alignment, wherein the alignment is calculated over the length of the sequence of described herein (preferably over the length of the shorter sequence). Sequence identity may preferably be calculated using the Needleman-Wunsch alignment algorithm (for example as implemented through the online server EMBOSS Needle (EMBOSS: the European Molecular Biology Open Software Suite. (2000) Trends in genetics. 16(6):276-7) and applying the following parameters: Matrix: DNAfull; Gap open penalty: 10.00; Gap extension penalty: 0.5; End Gap penalty: false; End Gap open penalty: 10.00; End Gap extension penalty: 0.5.

The term “linker” as used herein (for example as used in connection with the reporter proteins, heterologous amino acid sequences, binding moieties, or regulator moieties described herein) may be understood to mean any means of linking together (i.e., joining or conjugating) two proteins, peptides, or amino acid sequences. A linker preferably comprises or consists of an amino acid sequence, and may typically be between 1-20, 1-15, 1-10 or 1-5 amino acids in length, preferably between 1-10 amino acids in length. Typically, such linkers comprise glycine and serine residues, preferably at least 50%, 60%, 70%, 80%, 90% glycine and serine.

The term “species of”, e.g., species of heterologous amino acid sequence, protein, regulator moiety, binding moiety or target molecule, may be understood to refer to the type or kind of protein/moiety. For example, the same species of proteins typically have the same or closely relate amino acid sequences, or comprise the same protein domain. Thus the same species of proteins may have at least 95% sequence identity, preferably at least 98% sequence identity, to each other, as measured over the length of the shorter sequence. the same species of small molecule or ion, may have the same chemical identity or the same structure or a closely related structure, such that they are recognised as variants of the same molecular structure. For example the same species of molecules may share a common molecular structure, such as at least 90%, preferably 95%, most preferably at least 98%, of the same molecular structure. Different species are any two proteins/molecules that would not be recognised as the same species, according to these definitions. Each species may comprise a plurality of instances of the particular protein/molecule.

### Reporter Proteins

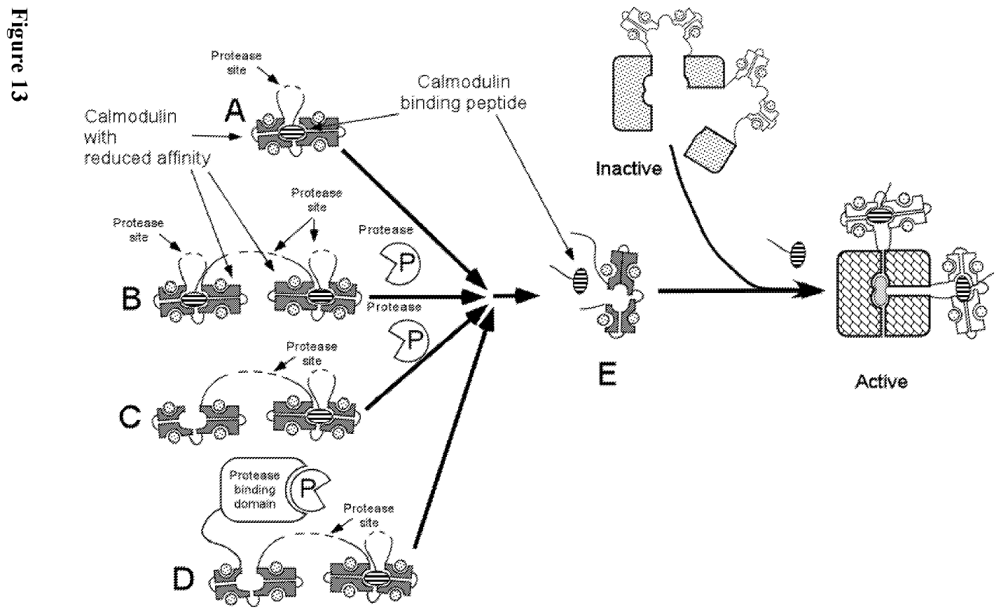

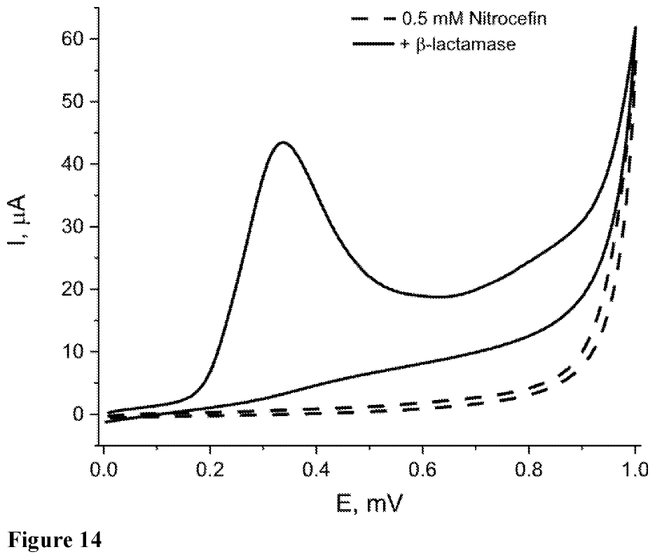

The reporter protein may be any protein capable of producing or generating a detectable and/or measurable signal. The reporter protein may be any protein capable of producing or generating a detectable and/or measurable signal, which is capable of being reversibly inactivated, such that conformational changes in the structure of the reporter protein reduce or eliminate the production or generation of the detectable and/or measurable signal. The reporter protein may be an enzyme, a fluorescent protein or a binding domain. The reporter protein is preferably an enzyme or a fluorescent protein, most preferably an enzyme. The detectable and/or measurable signal may be, for example, electrons, a change in redox state, protons (e.g., a change in pH), a colour change, antibiotic resistance, luminescence, fluorescence, and/or radioactivity. The detectable signal may be measured using electrochemical methods (see, e.g., FIG. 14), such that hydrolysis of an enzyme substrate to form a product may be measured electrochemically. A suitable electrochemical detection method, as would be well understood by the skilled person, may be measuring cyclic voltammograms, for example under the condition described in the description of FIG. 14. The electrochemical method may be capable of distinguishing between the substrate and the product, and optionally capable of detecting electrons generated during such a reaction. The electrochemical method may be capable of detecting electrons generated during conversion of a substrate to a product. The electrochemical method may be capable of being performed in a biological fluid such as serum. For example. β-lactamase activity may be detected using an electrochemical method to monitor hydrolysis of the substrate nitrocefin.

Typically, the enzyme, when catalytically active, is capable of reacting with or acting upon an enzyme substrate to thereby elicit a detectable signal. Non-limiting examples of suitable enzymes include trehalase, β-lactamase (ampicillin resistance protein), glucose dehydrogenase, an oxidoreductase or dehydrogenase (such as, e.g., flavin adenine dinucleotide-dependent glucose dehydrogenase (FADGDH), NAD-dependent glucose dehydrogenase (NAD-GDH), or pyranose dehydrogenase (PDH)), aminoglycoside phosphotransferase (kanamycin resistance protein), α-amylase, carbonic anhydrase, β-galactosidase, glucose oxidase, lysozyme, malate dehydrogenase, peroxidases (e.g., horseradish peroxidase (HRP)), phosphatases (e.g., alkaline phosphatase), luciferase, transferases, ATPases, nucleases (e.g., ribonucleases), kinases, synthases. Preferably, the enzyme is trehalase, β-lactamase, glucose dehydrogenase, an oxidoreductase or dehydrogenase (such as FADGDH or PDH), aminoglycoside phosphotransferase, a-amylase or carbonic anhydrase. Most preferably the enzyme is trehalase, β-lactamase, or glucose dehydrogenase. Non-limiting examples of suitable enzyme substrates include those that enable the generation of chromogenic, fluorescent, light (e.g., bioluminescent), electrical, pH, radioactive and other detectable signals such as for example antibiotic resistance when the enzyme is expressed in a suitable host cell such as a microbial cells, a yeast cell, or preferably a bacterial cell.

Typically, the fluorescent protein, when active, is capable of emitting light at a particular detectable wavelength, usually upon excitation at a particular wavelength. Non-limiting examples of suitable fluorescent proteins include mNeon, green fluorescent protein (GFP), yellow fluorescent protein (YFP), red fluorescent protein (RFP), Cherry, mCherry, mCFP, mTurquoise2, mOrange, mKate, dsRed, eqFP611, Dronpa, TagRFPs, KFP, EosFP/IrisFP, Dendra, bacterial phytochrome (BphP)-based fluorescent proteins (see, e.g., Matlashov, M. E., et al., Nat Commun 2020; 11, 239), or cyanobacteriochrome (CBCR)-derived fluorescent proteins (such as, e.g., miRFP670nano, see, e.g., Oliinyk O. S., et al., Nat Commun. 2019;10(1):279). BphP and CBCR-based fluorescent proteins may comprise a cofactor (such as biliverdin) for fluorescence. The fluorescent protein may be GFP or Cherry. The fluorescent protein may be mNeon, a BphP-based fluorescent protein or a CBCR-based fluorescent protein. The fluorescent protein may be mNeon. The reporter protein may be the fluorescent protein mNeon, in which case the detectable signal is the fluorescence emission of mNeon. An mNeon protein preferably comprises a sequence having at least 90%, optionally 100%, identity to the sequence of SEQ ID NO: 73, or a variant or functional fragment thereof. Variants and functional fragments typically retain fluorescent activity.

An enzyme is typically a protein capable of displaying catalytic activity towards a substrate molecule to thereby produce a detectable signal. The reporter protein may be the enzyme trehalase, in which case the substrate may be trehalose and the catalytic activity may be the conversion of trehalose to glucose. In the presence of an oxidoreductase or dehydrogenase enzyme, such as GDH, preferably a pyrroloquinoline quinone-GDH (PQQ-GDH), the glucose is converted to gluconolactone thereby producing electrons, in which case the detectable signal is the production of electrons. A trehalase preferably comprises a sequence having at least 90%, at least 91%, at least 92%, at least 93%, at least 94%, at least 95%, at least 96%, at least 97%, at least 98% or at least 99%, optionally 100%, identity to the sequence of SEQ ID NO: 1, or a variant or functional fragment thereof. The reporter protein may be the enzyme β-lactamase, in which case the substrate may be nitrocefin and the catalytic activity may be the degradation of the B-lactam ring of nitrocefin thereby resulting in a colour change from yellow to red, hence the detectable signal is the colour change from yellow to red. A β-lactamase preferably comprises a sequence having at least 90%, at least 91%, at least 92%, at least 93%, at least 94%, at least 95%, at least 96%, at least 97%, at least 98% or at least 99%, optionally 100%, identity to the sequence of SEQ ID NO: 3, or a variant or functional fragment thereof (for example SEQ ID NO: 4 or SEQ ID NO: 85). In some embodiments a β-lactamase is a thermostable β-lactamase, for example comprising a sequence having at least at least 98% or at least 99%, optionally 100%, identity to the sequence of SEQ ID NO: 85, The reporter protein may be the enzyme GDH, preferably PQQ-GDH, in which case the substrate may be glucose and the catalytic activity may be the conversion of glucose to gluconolactone thereby producing electrons, hence the detectable signal is the production of electrons. A PQQ-GDH preferably comprises a sequence having at least 90%, at least 91%, at least 92%, at least 93%, at least 94%, at least 95%, at least 96%, at least 97%, at least 98% or at least 99%, optionally 100%, identity to the sequence of SEQ ID NO: 5, or a variant or functional fragment thereof. A eGFP-GDH fusion protein may comprise a sequence having at least 90%. optionally 100%, identity to the sequence of SEQ ID NO: 78, or a variant or functional fragment thereof. The reporter protein may be the enzyme β-lactamase or the enzyme aminoglycoside phosphotransferase, in which case the substrate may be an antibiotic, such as ampicillin or kanamycin, respectively. The catalytic activity may be the degradation of the β-lactam ring of the antibiotic, and where the β-lactamase or the aminoglycoside phosphotransferase are expressed in a host cell, such a bacterial cell, the detectable signal may be to confer antibiotic resistance on the host cell (β-lactamase confers ampicillin resistance and aminoglycoside phosphotransferase confers kanamycin resistance). An aminoglycoside phosphotransferase preferably comprises a sequence having at least 90%, optionally 100%, identity to the sequence of SEQ ID NO: 8, or a variant or functional fragment thereof. The reporter protein may be the enzyme carbonic anhydrase, in which case the catalytic activity is typically the interconversion of carbon dioxide and water, and bicarbonate and hydrogen ions, hence the detectable signal is a change in pH. A carbonic anhydrase may comprise a sequence having at least 90%, optionally 100%, identity to the sequence of SEQ ID NO: 10, or a variant or functional fragment thereof. The reporter protein may be the enzyme α-amylase, in which case the catalytic activity is typically the hydrolysis of polysaccharides (e.g., amylose, starch, amylopectin, glycogen), most usually the hydrolysis of α-1,4 glycosidic linkages, to form intermediate oligosaccharides (e.g., dextrins), maltose and glucose.

An α-amylase may comprise a sequence having at least 90%, optionally 100%, identity to the sequence of SEQ ID NO: 83, or a variant or functional fragment thereof.

The reporter protein (preferably an enzyme) of the present invention comprises a first heterologous amino acid sequence which is responsive to binding of a first regulator moiety and a second heterologous amino acid sequence which is responsive to binding of a second regulator moiety. The reporter protein of the present invention may comprise more than one, optionally two or more, optionally a plurality of, heterologous amino acid sequences which are each responsive to binding a regulator moiety. The reporter protein of the present invention may comprise 1, 2, 3, 4, 5, 6, 7, 8, 9 or 10 heterologous amino acid sequences, optionally 1-5, preferably 1-3, most preferably 2, heterologous amino acid sequences, which are each responsive to binding a regulator moiety.

The first heterologous amino acid sequence may be provided as an insert within the amino acid sequence of the reporter protein and the second heterologous amino acid sequence may be provided as an insert within the amino acid sequence of the reporter protein. Preferably, the heterologous amino acid sequences are inserted at different locations in the amino acid sequence of the reporter protein. Each and preferably all of the heterologous amino acid sequences may be provided as an insert within (i.e., inserted into) the amino acid sequence of the reporter protein. Insertion of the two or more heterologous amino acid sequences in the amino acid sequence of the reporter protein typically reversibly inactivates the reporter protein, such that at least partial inactivation may be observed upon insertion of one heterologous amino acid sequences. Thus, insertion of two or more heterologous amino acid sequences in the amino acid sequence of the reporter protein may have an additive or epistatic effect.

Binding of a regulator moiety to each of (i.e., all of) the heterologous amino acid sequences reversibly regulates (preferably activates) the activity of the reporter protein (preferably the catalytic activity of the enzyme). Typically, binding of a first regulator moiety to the first heterologous amino acid sequence and binding of a second regulator moiety to the second heterologous amino acid sequence, reversibly activates the activity of the reporter protein. Typically binding of regulator moieties simultaneously at all of the heterologous amino acid sequences inserted in the reporter protein is required for full activation of the reporter protein. The reporter protein/enzyme is thus preferably engineered to be switchable from a state of reduced activity to a more active state, dependent on the binding of the regulator moieties. Typically binding of a regulator moiety to each and every heterologous amino acid sequence is required to fully activate the reporter protein, and in the absence of the regulator moieties the reporter protein typically does not exhibit any detectable activity.

It should be understood that wild-type activity may not be conferred on the reporter protein by binding of the regulator moieties. Typically, a reporter protein is active if a detectable signal, such as electrons, protons, light, fluorescence, antibiotic resistance, or a coloured product, is produced under appropriate reaction conditions and is able to be detected and/or measured. Where the reporter protein is an enzyme, the enzyme may be catalytically active if it is capable of displaying specific enzyme activity towards a substrate molecule to produce a detectable signal (such as electrons, protons, light, a colour change, a coloured product, antibiotic resistance or fluorescence), under appropriate reaction conditions. Where the reporter protein is a fluorescent protein, it may be active if it displays detectable fluorescence upon excitation at an appropriate wavelength or luminescence. As generally used herein, the term ‘inactive’ may refer to a reporter protein that is substantially incapable of displaying a detectable and/or measurable signal under appropriate reaction conditions. As generally used herein, the term ‘catalytically inactive’ may refer to an enzyme that is substantially incapable of displaying specific enzyme activity towards a substrate molecule under appropriate reaction conditions. Typically, the detectable signal (e.g., electrons, protons, light, a colour change, a coloured product, antibiotic resistance, or fluorescence) produced would be substantially less compared to that produced by a corresponding active reporter protein, or in the case where the reporter protein is an enzyme, as compared to that produced by a corresponding catalytically active enzyme, such as a corresponding wild-type enzyme. Production of the detectable signal (e.g., electrons, protons, light, a colour change, a coloured product, antibiotic resistance, or fluorescence) may be entirely absent for an inactive reporter protein, such as a reporter protein comprising two or more heterologous amino acid sequences.

The reporter proteins, preferably enzymes, and the biosensors comprising them, described herein, produce a detectable signal (such as electrons, protons, light, a colour change, a coloured product, antibiotic resistance, or fluorescence), by reacting with substrate molecules or an excitation wavelength, in response to binding, interacting with or otherwise detecting one or more target molecules. In this context react, reaction or reacting with a substrate molecule means enzymatically transforming the substrate molecule into one or more product molecules wherein the reaction produces a detectable signal or the product molecule may be directly or indirectly detected (for example the glucose product produced by trehalase may be indirectly detected by using for example a PQQ-GDH enzyme to convert the glucose into gluconolactone thereby producing electrons, which electrons may be detected). In this context react, reaction or reacting with an excitation wavelength means absorbing light at a particular excitation wavelength and emitting light at a different wavelength to produce a detectable fluorescence signal.

Where the detectable signal is electrons, the biosensor may act as an electron donor, whereby electrons produced by the reaction may flow either directly or via an electron shuttle (i.e., an electron mediator) such as, but not limited to, phenazine methosulfate or potassium ferrocyanide, to thereby act as an anode. The resulting change in potential between anode and cathode may be detected by an electronic detector. Where the reporter protein is an enzyme and the detectable signal is the production of electrons, the reporter protein or biosensor may be attached to an electrode. The mode of attachment may permit direct electron transfer from the oxidoreductase enzyme or biosensor to the electrode. Typically, the biosensor or enzyme acts as an electron donor and electrons produced by the reaction may flow directly to the electrode to form the anode. The electrode may be composed of gold, platinum, carbon nanotubes or graphene. The enzyme or biosensor may be attached to the electrode surface using 1-pyrenebutanoic acid succinimidyl ester (PBSE) as a hetero-bifunctional linker, wherein the active ester groups of the PBSE linker may react with the amino groups of lysine residues in the oxidoreductase enzyme or biosensor. The electrode may be a screen printed electrode layered with a dry mixture comprising the oxidoreductase enzyme and/or biosensor of the invention and preferably further comprising an electron mediator. The enzyme may be immobilised on the electrode via a modified co-factor (such as PQQ for example). The modified co-factor may be functionalised with a linker that is attached to the surface of the electrode either covalently or non-covalently, for example through an attached group (for example through a pyrene-carbon nanotube interaction.

The reporter proteins, enzymes, fluorescent proteins, or biosensors described herein may be lyophilised (i.e., freeze-dried, cryodessicated), for example by using a typical low temperature dehydration process that is well-known in the art. The reporter proteins, enzymes, fluorescent proteins, or biosensors described herein may be lyophilised and re-hydrated (i.e., reconstituted) and retain their activity, i.e., do not show a significantly reduced activity after lyophilisation and re-hydration, as compared to their activity prior to lyophilisation. The reporter proteins, enzymes, fluorescent proteins, or biosensors described herein may be lyophilised, re-hydrated and stored at room temperature for at least 7 days and retain their activity.

### Heterologous Amino Acid Sequence

Described herein is a reporter protein (preferably an enzyme) comprising two or more heterologous amino acid sequences. Described is reporter protein (preferably an enzyme) comprising a first heterologous amino acid sequence which is responsive to binding of a first regulator moiety and a second heterologous amino acid sequence which is responsive to binding of a second regulator moiety, wherein binding of the first regulator moiety to the first heterologous amino acid sequence and binding of the second regulator moiety to the second heterologous amino acid sequence, reversibly regulates the activity of the reporter protein. The reporter protein may further comprise one or more further heterologous amino acid sequences.

The heterologous amino acid sequences may be provided as inserts within the amino acid sequence of the reporter protein. Preferably, all of the heterologous amino acid sequences comprised in the reporter protein are provided as inserts within the amino acid sequence of the reporter protein. In some instances, the first heterologous amino acid sequence is provided as an insert within the amino acid sequence of the enzyme and the second heterologous amino acid sequence is provided as an insert within the amino acid sequence of the reporter protein. However, fusions of a heterologous amino acid sequence at the N- and/or C-terminus of the amino acid sequence of the reporter protein are also possible. Preferably, the reporter protein amino acid sequence and the two or more heterologous amino acid sequences (e.g., the first and the second heterologous amino acid sequences) are present in, or form at least part of a single, contiguous amino acid sequence.

When provided as an insert, the two or more (i.e., first and second and any further) heterologous amino acid sequences are contiguous with, respective portions, sub-sequences or fragments of the reporter protein. The two or more heterologous amino acid sequences may be inserted at the same location within the amino acid sequence of the reporter protein, for example in series (i.e., as a contiguous sequence, one after the other), with the C-terminus of one heterologous amino acid sequence linked to the N-terminus of the next heterologous amino acid sequence in the series. However, preferably, each heterologous amino acid sequence is inserted within the amino acid sequence of the reporter protein at a different insertion location. The insertion is typically made at a position in the amino acid sequence of the reporter protein which tolerates said insertion without steric clashes preventing stable folding of the reporter protein. Preferably, the heterologous amino acid sequences are inserted into the amino acid sequence of the reporter protein at a solvent exposed portion of the reporter protein. The heterologous amino acid sequences may be inserted into any of an a-helix, a B-sheet or a loop in the amino acid sequence of the reporter protein, preferably in the proximity of the active site; typically in the order of preference of insertion site location being loops most preferred, followed by B-sheets, followed by a-helices being least preferred. In the proximity of the active site may be understood to mean any site at which a perturbation in structure results in a structural change in the conformation of the amino acid residues making up the active site of the reporter protein (i.e., the site responsible for the activity producing the detectable or measurable signal). Thus, insertions may be made within 5 Å, 10 Å, 15 Å, 20 Å, 25 Å or within 50 Å, distance from the closest active site residue, preferably within 20 Å, most preferably within 10 Å. Each heterologous amino acid sequence may be provided as an insert within the amino acid sequence of the reporter protein flanked on either side by linkers. The linkers may be added between each heterologous amino acid insert and the sequence of the reporter protein to assist toleration of the insertion. Similarly, if two or more heterologous amino acid sequences are linked in series, they may be connected through linkers. The linkers may be as described herein.

The reporter protein may comprise each heterologous amino acid sequence at a loop or turn region in the structure of the reporter protein, which functionally tolerates insertion of a heterologous amino acid sequence. The reporter protein may comprise the two or more heterologous amino acid sequences at one or more locations in the reporter protein (such as a loop or turn) which comprise one or more amino acid residues which influence activity of the reporter protein. Where the reporter protein is an enzyme, the heterologous amino acid sequence inserts may thus displace one or more residues which influence substrate binding and/or catalytic activity of the enzyme, such that catalytic activity of the enzyme is regulated by the two or more heterologous amino acid sequences (e.g., the first and second, and optionally any further, heterologous amino acid sequences). Where the reporter protein is an enzyme, each heterologous amino acid sequence insert may displace one more residues which influence substrate binding and/or one or more residues which influence catalytic activity. Where the reporter protein is an enzyme, each heterologous amino acid sequence insert may prevent or reduce substrate binding to the enzyme and/or may switch the enzyme to a state of reduced catalytic activity or a catalytically inactive state. Where the reporter protein is a fluorescent protein, the heterologous amino acid sequence inserts may thus displace one or more residues which influence structure of the fluorophore of the fluorescent protein, such that fluorescence of the fluorescent protein is regulated by the two or more heterologous amino acid sequences. Where the reporter protein is a fluorescent protein, each heterologous amino acid sequence insert may prevent, or reduce the level of, fluorescence capable of being emitted by the fluorescent protein.

A heterologous amino acid sequence may be a protein. Preferably all of the heterologous amino acid sequences are proteins. Typically, each of the heterologous amino acid sequences are proteins that are capable of undergoing a conformational change in response to binding of a regulator moiety. Each heterologous amino acid sequence may comprise one more domains (such as one or two domains) which undergo structural rearrangement (i.e., conformational change) upon binding of a regulator moiety. Each heterologous amino acid sequence may comprise an unstructured or unfolded amino acid sequence which undergoes a structural rearrangement or conformational change upon binding of a regulator moiety, which optionally creates one or more folded protein domains. Each heterologous amino acid sequence may be a protein that undergoes a structural rearrangement or conformational change upon binding of a regulator moiety that changes its interaction with the reporter protein. For example, binding of a regulator moiety may induce a structural rearrangement in the heterologous amino acid sequence, forming a binding site for the reporter protein, such that the reporter protein and the heterologous amino acid sequence associate, thereby regulating the activity of the reporter protein. Preferably, each heterologous amino acid sequence is a protein that undergoes a structural rearrangement or conformational change upon binding of a regulator moiety that increases or decreases the distance in space between the N- and C-termini of the heterologous amino acid sequence. Thus, binding of a regulator moiety to a heterologous amino acid sequence may cause a conformational change in the structure of the heterologous amino acid sequence. Each heterologous amino acid sequence may be a different protein, but preferably each of the heterologous amino acid sequences is the same protein, or a functional variant or fragment thereof. For example, the first and second heterologous amino acid sequences may be different proteins, or preferably may be the same protein, or a functional variant or fragment thereof. The first heterologous amino acid sequence may be a first species of protein. or a functional fragment thereof, and the second heterologous amino acid sequence may be second species of protein, or a functional fragment thereof. In some instances, at least one (preferably all) of the heterologous amino acid sequences is a calmodulin protein, or a variant or functional fragment thereof. In some instances, at least one (preferably all) of the heterologous amino acid sequences is an affinity clamp, as further described herein. In some instances, at least one (preferably all) of the heterologous amino acid sequences is a solute binding protein, as further described herein. The first heterologous amino acid sequence may be a calmodulin protein and the second heterologous amino acid sequence may be an affinity clamp. Preferably all of the heterologous amino acid sequences (e.g., the first and second, and optionally any further, heterologous amino acid sequences) are each a calmodulin protein, or a functional fragment thereof. In preferred instances, at least one, preferably each, of the heterologous amino acid sequences is a calmodulin protein, its engineered variant, or a functional fragment thereof.

At least one, preferably all (e.g. the first and second), of the heterologous amino acid sequences may be a calcium-binding protein, or a functional fragment thereof. At least one, preferably all (e.g. the first and second), of the heterologous amino acid sequences may be a calmodulin protein, or a functional fragment thereof. At least one, preferably all (e.g. the first and second), of the heterologous amino acid sequences may be an affinity clamp (e.g., an ePDZ domain). At least one, preferably all (e.g. the first and second), of the heterologous amino acid sequences may be a solute binding protein (SBP) (also known as, periplasmic binding proteins (PBPs) or Venus-fly trap proteins, see e.g., Edwards, K. A., Talanta Open, 2021, 3:100038). At least one, preferably all (e.g. the first and second), of the heterologous amino acid sequences may be an SH3 domain. At least one, preferably all (e.g. the first and second), of the heterologous amino acid sequences may be an antibody. At least one, preferably all (e.g. the first and second), of the heterologous amino acid sequences may be a leucine zipper peptide. The reporter protein comprises two or more heterologous amino acid sequences (e.g., a first and a second heterologous amino acid sequence), and hence there may be a single species of heterologous amino acid sequence, where each heterologous amino acid sequence is the same. Or there may be a plurality of different species of heterologous amino acid sequences.

The reporter protein may comprise 2, 3, 4, 5, 6, 7, 8. 9, 10, or more, heterologous amino acid sequences. The reporter protein may comprise from 2 to 10 heterologous amino acid sequences. The reporter protein may comprise from 2 to 5 heterologous amino acid sequences. Preferably, the reporter protein may comprise 2 or 3 heterologous amino acid sequences, which may each be the same or different. Most preferably, the reporter protein comprises two heterologous amino acid sequences, which may the same or different. Thus, the reporter protein may comprise a plurality of different species of heterologous amino acid sequence, for example 2, 3, 4, 5, 6, 7, 8, 9, 10, or more, species of heterologous amino acid sequence. Preferably, the reporter protein comprises one or two different species of heterologous amino acid sequence. Most preferably the reporter protein comprises a single species of heterologous amino acid sequence. Thus, in some preferred instances, the reporter protein comprises a first and a second heterologous amino acid sequence, which are the same species of heterologous amino acid sequence, i.e., they are the same. The heterologous amino acid sequence may be selected from a calmodulin protein or a functional fragment thereof; an affinity clamp (e.g., an ePDZ domain); an SBP; an SH3 domain; an antibody; a leucine zipper peptide; or combinations thereof.

Typically, at least one, preferably all (i.e., the first and second, and optionally any further), of the heterologous amino acid sequences is an amino acid sequence of a calcium-binding protein, or a functional fragment thereof. Where at least one heterologous amino acid sequence is a calcium-binding protein or functional fragment thereof, regulation of activity of the reporter protein typically requires the presence of calcium ions. In preferred instances, at least one, or preferably all (i.e., the first and second, and optionally any further), of the heterologous amino acid sequences comprises, or preferably is, a calmodulin protein or a functional fragment thereof. The calmodulin protein or functional fragment thereof may be calcium-insensitive. A “calmodulin protein” as described herein may be any calmodulin protein or domain previously described and variants thereof, and includes derivatives and variants of specific calmodulin proteins or domains described herein. At least one, or preferably all, of the heterologous amino acid sequences may comprise or consist of a calmodulin protein comprising a sequence having at least 60%, 70%, 80%, 85%, 90%, 95%, 96%, 97%, 98%, 99% or at least 100% sequence identity, optionally at least 80%, preferably at least 90%, most preferably at least 95% sequence identity to any one of SEQ ID NOs: 11, 12, 15 or 16, preferably SEQ ID NO: 11, or a variant or functional fragment thereof. At least one, or preferably each, of the heterologous amino acid sequences may comprise or consist of the sequence of any one of SEQ ID NOs: 11, 12. 15 or 16, preferably SEQ ID NO: 11, or a variant or functional fragment thereof. An exemplary wild-type calmodulin protein is shown in SEQ ID NO: 15. Variants and functional fragments are described herein and in this context typically retain calmodulin activity. For example, variants and functional fragments typically retain calcium-binding activity and/or calmodulin-binding peptide (CaM-BP)-binding activity, preferably both activities. Variants and functional fragments may not have calcium-binding activity and/or be calcium-insensitive; such variants and functional fragments typically retain ‘calmodulin-binding peptide’ binding activity. Where a calmodulin protein or functional fragment thereof is calcium-insensitive, it may bind calcium, but such calcium-binding may not significantly affect the conformation of the calmodulin protein or functional fragment thereof. In some instances, at least one, preferably each, of the heterologous amino acid sequences may comprise or consist of a calmodulin protein or functional fragment thereof comprising one or more modifications resulting in a reduced binding affinity for a calmodulin-binding peptide (CaM-BP). Such calmodulin proteins having a reduced binding affinity for a CaM-BP may comprise or consist of a sequence having at least 60%, 70%, 80%, 85%, 90%, 95%, 96%, 97%, 98%, 99% or at least 100% sequence identity, optionally at least 80%, preferably at least 90%, most preferably at least 95% sequence identity to SEQ ID NO: 16, or a variant or functional fragment thereof. Such calmodulin proteins having a reduced binding affinity for a CaM-BP may comprise or consist of SEQ ID NO: 16, or a variant or functional fragment thereof.

Where at least one, or preferably all, of the heterologous amino acid sequences is a calmodulin protein, or a functional fragment thereof, the activity of the reporter protein may be capable of being regulated (i.e., by binding of regulator moieties to each of the heterologous amino acid sequences) in the presence of physiological calcium. Thus, typically, it is binding of a regulator moiety to the calmodulin protein that induces a conformational change in the calmodulin protein, rather than the binding of calcium. Thus, typically the regulator moiety is not calcium. Physiological calcium concentrations are typically between 500 μM to 5 mM calcium (i.e., Ca²⁺), preferably from 1 mM to 2 mM calcium. In preferred instances, binding of a regulator moiety to each of the heterologous amino acid sequences reversibly regulates activity of the reporter protein in the presence of at least 1 mM calcium, preferably in the presence of 1 mM to 2 mM calcium. In some instances, changes in the calcium concentration between 0.5-5 mM, preferably 1 mM to 2 mM, do not appreciably affect the activity of the reporter protein. Thus, the activity of the reporter enzyme is typically not dependent on calcium concentration, where the calcium concentration is within physiological levels.

Typically, at least one, preferably all (i.e., the first and second, and optionally any further), of the heterologous amino acid sequences is an amino acid sequence of an affinity clamp, or a functional fragment thereof. In preferred instances, at least one, or preferably all (i.e., the first and second, and optionally any further), of the heterologous amino acid sequences comprises, or preferably is, an affinity clamp. An “affinity clamp” as described herein may be any affinity clamp previously described and variants thereof. At least one, or preferably all, of the heterologous amino acid sequences may comprise or consist of an affinity clamp comprising a sequence having at least 60%, 70%, 80%, 85%, 90%, 95%, 96%, 97%, 98%, 99% or at least 100% sequence identity, optionally at least 80%, preferably at least 90%, most preferably at least 95% sequence identity to SEQ ID NO: 48 or 76, preferably SEQ ID NO: 76, or a variant or functional fragment thereof. At least one, or preferably each, of the heterologous amino acid sequences may comprise or consist of the sequence of SEQ ID NO: 76, or a variant or functional fragment thereof. Variants and functional fragments typically retain the ability to bind the affinity clamp ligand, which may preferably be an RGS peptide ligand. An exemplary RGS peptide ligand may comprise, or consist of, the sequence of SEQ ID NO: 82. Thus, at least one, or preferably all (i.e., the first and second, and optionally any further), of the heterologous amino acid sequences may comprise, or preferably consist of, an affinity clamp, optionally comprising the sequence of SEQ ID NO: 76, which is capable of binding to a peptide ligand, where said peptide ligand optionally comprises the sequence of SEQ ID NO: 82. Typically, binding of a peptide ligand to the affinity clamp induces a conformational change in the affinity clamp, an activity which is typically retained by variants and functional fragments.

### Regulator Moieties

A regulator moiety is any moiety capable of binding to a heterologous amino acid sequence as defined herein. Typically, binding of a regulator moiety to a heterologous amino acid sequence causes or induces a conformational change or structural rearrangement in the heterologous amino acid sequence. Thus, the nature or identity of the regulator moiety typically depends on the nature or identity of the heterologous amino acid sequence. Thus, where there are two different species of heterologous amino acid sequence (e.g., the first and second heterologous amino acid sequences are different), there may be two different species of regulator moiety. Where there are two or more heterologous amino acid sequences which are the same species of heterologous amino acid sequence (e.g., the first and second heterologous amino acid sequences are the same), there is typically one species of regulator moiety. The regulator moiety for the first heterologous amino acid sequence may be the same as or different from, preferably the same as, the regulator moiety for the second heterologous amino acid sequence. The regulator moiety for the first heterologous amino acid sequence may be linked to the regulator moiety for the second heterologous amino acid sequence.

A regulator moiety may be any ligand, analyte, small organic molecule, ion, epitope, domain, fragment, subunit, moiety or combination thereof. A regulator moiety may be a peptide or a protein, including antibodies and antibody fragments, antigens, enzymes, phosphoproteins, glycoproteins, lipoproteins and glycoproteins. A regulator moiety may be a lipid, phospholipid, carbohydrate (including simple sugars, disaccharides and polysaccharides), nucleic acid, nucleoprotein or any other molecule or analyte. A regulator moiety may be a small molecule, such as a drug or other pharmaceutical including an antibiotic. A regulator moiety may be anything that induces a conformational change in the heterologous amino acid sequence, or otherwise facilitates interaction between the heterologous amino acid sequence and the reporter protein, so as to activate the reporter protein. Thus, a regulator moiety may be a modification, such as a covalently-added post-translational modification (e.g., phosphorylation, glycosylation, sumoylation, lipidation, etc.), proteolytic cleavage (e.g., by a protease) or ligation (e.g., formation of disulfide bonds, for example to create a redox sensor). A regulator moiety may be a peptide or a small molecule.

Preferably, the regulator moiety is a peptide. Preferably the first and the second regulator moieties are peptides. The peptide may have any sequence, but is capable of binding to the heterologous amino acid sequence. Thus, the regulator moiety may comprise, or consist of, a peptide that is capable of binding to a heterologous amino acid sequence as defined herein. Typically, binding of said peptide to a heterologous amino acid sequence causes a conformational change or structural rearrangement in the heterologous amino acid sequence. Thus, where the regulator moiety is a peptide (i.e., a regulator peptide), the sequence of the peptide typically depends on the nature or identity of the heterologous amino acid sequence. Thus, where there are two or more different species of heterologous amino acid sequence (e.g., the first and second heterologous amino acid sequence are different), there may be two or more different species of regulator peptide (e.g., the first and second regulator moieties are different). Where there are two or more heterologous amino acid sequences which are the same species or type of heterologous amino acid sequence (e.g., the first and second heterologous amino acid sequence are the same), there is typically one species or type of regulator peptide (e.g., the first and second regulator moieties are the same).

Where at least one of the heterologous amino acid sequences is a calmodulin protein, or a functional fragment thereof, at least one of the regulator moieties may be a calmodulin-binding peptide, or variant thereof. Where all of the (i.e., the first and second, and optionally any further) heterologous amino acid sequences are each calmodulin proteins, or functional fragments thereof, the regulator moieties (i.e., the first and second, and optionally any further regulator moieties) are typically calmodulin-binding peptides, or variants thereof. Thus, in preferred instances, the regulator moiety is a calmodulin-binding peptide, or variant thereof. In some instances, the first and second (and optionally any further) heterologous amino acid sequences are each a calmodulin protein, or variant or functional fragment thereof, and the first and second (and optionally any further) regulatory moieties are each a calmodulin-binding peptide, or variant thereof, and wherein binding of a calmodulin-binding peptide to a calmodulin protein activates the activity of the reporter protein.

A calmodulin-binding peptide (CaM-BP) preferably comprises any amino acid sequence (preferably a linear peptide epitope of 1-20 amino acids) that is capable of specifically binding to calmodulin (i.e., a calmodulin protein, or a variant or functional fragment thereof as defined herein) and preferably inducing a structural rearrangement or conformational change in the calmodulin that brings the N- and C-termini of calmodulin into proximity. Preferably, a CaM-BP is capable of binding to a heterologous amino acid sequence, which is a calmodulin protein or a functional fragment thereof, that is provided as an insert within the amino acid sequence of a reporter protein. Such binding typically reversibly regulates, preferably activates, activity of the reporter protein, as described further herein. A “CaM-BP” may be any previously described CaM-BP (see, e.g., Kursula, P., Amino Acids (2014), 46 (10): 2295-304), or variants thereof, and includes derivatives or variants of specific CaM-BPs described herein. The CaM-BP may comprise a sequence having at least 60%, 70%, 80%, 85%, 90%, 95%, 96%, 97%, 98%, 99% or at least 100% sequence identity to, optionally at least 80% sequence identity to, preferably at least 90% sequence identity to, most preferably at least 95% sequence identity to any one of SEQ ID NOs: 17-35, preferably any one of SEQ ID NOs: 17, 18 or 19, most preferably SEQ ID NO: 17. The CaM-BP may comprise a contiguous sequence of at least 5, 6, 7, 8, 9, or 10, optionally at least 5, preferably at least 10, amino acids found in any one of SEQ ID NOs: 17-35, preferably any one of SEQ ID NOs: 17, 18 or 19, most preferably SEQ ID NO: 17, which is capable of binding calmodulin. The CaM-BP may comprise or consist of the sequence of any one of SEQ ID NOs: 17-35, preferably any one of SEQ ID NOs: 17, 18 or 19, most preferably SEQ ID NO: 17, or variants thereof. Preferably, the CaM-BP may comprise or consist of the sequence of any one of SEQ ID NOs: 17, 18 or 19, most preferably SEQ ID NO: 17, or a variant thereof. Variants typically comprise sequences having deletions or insertions of between 1-5 amino acids relative to the sequence of any one of SEQ ID NOs: 17-35, preferably any one of SEQ ID NOs: 17, 18 or 19, most preferably SEQ ID NO: 17. Variants preferably comprise sequences comprising between 1-10 mutations, preferably 1-5 mutations, relative to the sequence of any one of SEQ ID NOs: 17-35, preferably any one of SEQ ID NOs: 17, 18 or 19, most preferably SEQ ID NO: 17. Variants retain binding to calmodulin. In some instances the CaM-BP may have a reduced binding affinity for calmodulin, i.e., the CaM-BP may have a binding affinity for a calmodulin protein that is lower than or reduced as compared to the binding affinity of wild type CaM-BP for said calmodulin protein. An exemplary wild type CaM-BP is a peptide having the sequence of SEQ ID NO: 17. An exemplary CaM-BP having an increased binding affinity for calmodulin, as compared to a wild type CaM-BP, may have the sequence of SEQ ID NO: 18. A CaM-BP having a reduced binding affinity for calmodulin may preferably comprise, or consist essentially of, the sequence of SEQ ID NO: 19, or a variant thereof. An exemplary CaM-BP having a reduced binding affinity for calmodulin, as compared to a wild type CaM-BP, may have the sequence of SEQ ID NO: 19.

Where at least one of the heterologous amino acid sequences is an affinity clamp, or a functional fragment thereof, at least one of the regulator moieties may be an affinity clamp-binding peptide ligand (such as an RGS peptide ligand), or variant thereof. Where all of the (i.e., the first and second, and optionally any further) heterologous amino acid sequences are each affinity clamps, or functional fragments thereof, the regulator moieties (i.e., the first and second, and optionally any further regulator moieties) are typically affinity clamp-binding peptide ligands (such as RGS peptide ligands), or variants thereof. Thus, the regulator moiety may be an affinity clamp-binding peptide ligand (such as an RGS peptide ligand), or variant thereof. In some instances, the first and second (and optionally any further) heterologous amino acid sequences are each an affinity clamp, or variant or functional fragment thereof, and the first and second (and optionally any further) regulatory moieties are each an affinity clamp-binding peptide ligand (such as an RGS peptide ligand), or variant thereof, and wherein binding of an affinity clamp-binding peptide ligand (such as an RGS peptide ligand) to an affinity clamp activates the activity of the reporter protein.

An affinity clamp-binding peptide ligand (such as an RGS peptide ligand) preferably comprises any amino acid sequence (preferably a linear peptide epitope of 1-20 amino acids) that is capable of specifically binding to an affinity clamp as defined herein, and preferably inducing a structural rearrangement or conformational change in the affinity clamp. Preferably, an affinity clamp-binding peptide ligand (such as an RGS peptide ligand) is capable of binding to a heterologous amino acid sequence, which is an affinity clamp or a functional fragment thereof, that is provided as an insert within the amino acid sequence of a reporter protein. Such binding typically reversibly regulates, preferably activates, activity of the reporter protein, as described further herein. An affinity clamp-binding peptide ligand (such as an RGS peptide ligand) may comprise a sequence having at least 60%, 70%, 80%, 85%, 90%, 95%, 96%, 97%, 98%, 99% or at least 100% sequence identity to, optionally at least 80% sequence identity to, preferably at least 90% sequence identity to, most preferably at least 95% sequence identity to SEQ ID NO: 82. The affinity clamp-binding peptide ligand (such as an RGS peptide ligand) may comprise a contiguous sequence of at least 3, 4, 5, 6, 7, or 8, optionally at least 3, preferably at least 5, amino acids found in SEQ ID NO: 82, which is capable of binding the affinity clamp. The affinity clamp-binding peptide ligand (such as an RGS peptide ligand) may comprise or consist of the sequence of SEQ ID NO: 82, or variants thereof. Variants typically comprise sequences having deletions or insertions of between 1-5 amino acids, preferably 1 or 2 or 3, most preferably 1 or 2 amino acids, relative to the sequence of SEQ ID NO: 82. Variants preferably comprise sequences comprising between 1-5 mutations, preferably 1-2 mutations, relative to the sequence of SEQ ID NOs: 82. Variants retain binding to the affinity clamp (e.g., SEQ ID NO: 76).

The regulator moiety may be a peptide capable of binding an affinity clamp (e.g., a PDZ domain binding peptide), in which case at least one of the heterologous amino acid sequences may be an affinity clamp (e.g., an ePDZ domain). The regulator moiety may be an SH3 domain binding peptide, in which case at least one of the heterologous amino acid sequence may be an SH3domain). The regulator moiety may be an antibody binding peptide or antigen, in which case at least one of the heterologous amino acid sequence may be an antibody. The regulator moiety may be a leucine zipper peptide in which case at least one of the heterologous amino acid sequence may be a second leucine zipper peptide. The regulator moiety may be a small molecule, solute or peptide capable of binding a solute binding protein, as further described herein,, in which case at least one of the heterologous amino acid sequences may be a solute binding protein. There may be a plurality of different species of regulator moiety depending on the number of different species of heterologous amino acid sequences comprised in the reporter protein. Thus, the regulator moiety may be selected from a calmodulin-binding peptide, or variant thereof; a peptide binding an affinity clamp; an SH3 domain binding peptide; an antibody binding peptide; a leucine zipper peptide; a small molecule, solute or peptide binding a solute binding protein; or combinations thereof.

The peptide may be between 1-10, 1-15, 1-20, 1-30, 1-40, 1-50, 1-100, 1-200 amino acids in length, preferably between 1-20 amino acids in length. The peptide may comprise or consist of a linear binding epitope that binds a heterologous amino acid sequence. In some instances, the regulator moiety is a peptide that binds to a calmodulin protein, or a functional fragment thereof. In preferred instances, the first regulator moiety is a CaM-BP and the second regulator moiety is CaM-BP. Typically, binding of a first CaM-BP to the first heterologous amino acid sequence which is a calmodulin protein or fragment thereof, and binding of a second CaM-BP to the second heterologous amino acid sequence which is a calmodulin protein or fragment thereof, activates the activity of the reporter protein.

The first and second regulator moieties may both be peptides, and the regulator moiety for the first heterologous amino acid sequence may have the same sequence as, or a different sequence from (preferably the same sequence as), the regulator moiety for the second heterologous amino acid sequence. The first and second regulator moieties may both be peptides, and the first regulator moiety for the first heterologous amino acid sequence may be linked to the second regulator moiety for the second heterologous amino acid sequence, for example through a linker (such as a peptide linker, as further defined herein).

The regulator moiety may be a ‘caged’ regulator moiety, for example the regulator moiety may be linked to a protein that is capable of binding to, and thereby sequestering, the regulator moiety, such that the regulator moiety is not free to bind a heterologous amino acid sequence on the reporter protein. In such cases, the regulator moiety is preferably a peptide. The regulator moiety may be linked to a protein that corresponds to a variant of the heterologous amino acid sequence on the reporter protein, but which has a lower binding affinity for the regulator moiety, as compared to the heterologous amino acid sequence on the reporter protein. The regulator moiety may be linked to said protein through a linker, which is preferably a peptide linker. Thus, the caged regulator moiety may be fusion protein comprising the peptide regulator moiety, linked through a peptide linker, to the binding/sequestering protein. The caged regulator moiety may preferably comprise a calmodulin-binding peptide (CaM-BP) (such as, e.g., any one of SEQ ID NOs: 17-35, preferably any one of SEQ ID NOs: 17, 18 or 19, most preferably SEQ ID NO: 17) linked (optionally through a peptide linker) to a calmodulin protein having a reduced binding affinity for CaM-BP, as described herein (e.g., SEQ ID NO: 16), this is typically the case when the first and second heterologous amino acid sequences on the reporter protein are calmodulin protein inserts. The first and second regulator moieties may both be caged regulator moieties, and optionally the first regulator moiety for the first heterologous amino acid sequence may be linked to the second regulator moiety for the second heterologous amino acid sequence, for example through a linker.

In some instances, the peptide linker connecting the regulator moiety and the binding/sequestering protein may comprise a protease cleavage site. The caged regulator moiety may preferably comprise a calmodulin-binding peptide (CaM-BP) (such as, e.g., any one of SEQ ID NOs: 17-35, preferably any one of SEQ ID NOs: 17, 18 or 19, most preferably SEQ ID NO: 17) linked through a peptide linker comprising a protease cleavage site to a calmodulin protein having a reduced binding affinity for CaM-BP, as described herein (e.g., SEQ ID NO: 16); this is typically the case when the first and second heterologous amino acid sequences on the reporter protein are calmodulin protein inserts. The caged regulator moiety may comprise two CaM-BPs and two calmodulin proteins having a reduced binding affinity for CaM-BP, wherein the two calmodulin proteins are linked through a peptide linker, and each calmodulin protein is linked to a respective CaM-BP through a peptide linker, wherein each of the linkers comprises a protease cleavage site. The caged regulator moiety may comprise one CaM-BP and two calmodulin proteins having a reduced binding affinity for CaM-BP, wherein the two calmodulin proteins are linked through a peptide linker, and one of the calmodulin protein is linked to the CaM-BP through a peptide linker, wherein each of the linkers comprises a protease cleavage site. The caged regulator moiety may comprise one CaM-BP, two calmodulin proteins having a reduced binding affinity for CaM-BP. and a protease binding domain that specifically binds a protease; wherein the two calmodulin proteins are linked through a peptide linker comprising a target cleavage site of the protease, one of the calmodulin protein is linked to the CaM-BP through a peptide linker comprising a target cleavage site of the protease, and the other calmodulin protein is linked to the protease binding domain, optionally wherein the protease binding domain is bound to the protease. Such caged regulatory moieties reduce the background activation of the reporter protein/biosensor. Such caged regulatory moieties comprising protease cleavage sites, may be used in a biosensor for detecting a protease, wherein the protease cleavage sites are target cleavage sites of the protease of interest.

### Binding Moieties

The reporter proteins described herein are typically configured to be activated in the presence of one or more target molecules (i.e., one or more species of target molecule). Thus, the reporter protein further comprises at least one, preferably two, binding moieties. The reporter protein may comprise a binding moiety (B1′). The reporter protein may comprise a first binding moiety (B1′) and a second binding moiety (B2′). A binding moiety on the reporter protein typically interacts with and/or binds a target molecule of the biosensor. Typically each regulator moiety comprises at least one, preferably one, binding moiety. A binding moiety on a regulator moiety may interact with and/or bind a target molecule of the biosensor. Each binding moiety of the reporter protein typically forms an interacting pair with a respective binding moiety on a regulator moiety. Thus, each regulator moiety may comprise a binding moiety that is capable of interacting with at least one of the binding moieties on the reporter protein. A regulator moiety may comprise a binding moiety (B1″), which is capable of interacting with a binding moiety (B1′) on the reporter protein. The first regulator moiety may comprise a binding moiety (B1″) which is capable of interacting with the first binding moiety (B1′) on the reporter protein, and the second regulator moiety may comprise a binding moiety (B2″) which is capable of interacting with the second binding moiety (B2′) on the reporter protein. Preferably, the binding moiety on the first regulator moiety (B1″) forms an interacting pair with the first binding moiety on the reporter protein (B1′), and the binding moiety on the second regulator moiety (B2″) forms an interacting pair with the second binding moiety on the reporter protein (B2′). The first regulator moiety and the second regulator moiety may be linked (for example through a peptide linker, optionally as a fusion protein) and may further comprise a binding moiety (B1″), which is capable of interacting with a binding moiety (B1′) on the reporter protein.

Each pair of binding moieties may interact directly, i.e., bind directly, to each other. Preferably, each pair of binding moieties interact through binding to a target molecule. Thus, interaction between a binding moiety on the reporter protein and a binding moiety on a regulator moiety may be dependent on the presence of a target molecule. Each pair of binding moieties may bind at the same time (i.e., simultaneously) to the same target molecule. A binding moiety (B1′) on the reporter protein and a binding moiety (B1″) on a regulator moiety may both bind a target molecule (TM1) simultaneously. Alternatively, a binding moiety (B1′) on the reporter protein may bind a target molecule (TM1) and a binding moiety (B1″) on a regulator moiety may bind to the complex of B1′ and TM1; or a binding moiety (B1″) on a regulator moiety may bind a target molecule (TM1) and a binding moiety (B1′) on the reporter protein may bind to the complex of B1″ and TM1. The first binding moiety on the reporter protein (B1′) and the binding moiety of the first regulator moiety (B1″) may both bind a first target molecule (TM1) simultaneously; and the second binding moiety on the reporter protein (B2′) and the binding moiety on the second regulator moiety (B2″) may both bind a second target molecule (TM2) simultaneously. The first binding moiety on the reporter protein (B1′) may bind a first target molecule (TM1) and the binding moiety of the first regulator moiety (B1″) may bind the complex of B1′ and TM1, or the binding moiety of the first regulator moiety (B1″) may bind a first target molecule (TM1) and the first binding moiety on the reporter protein (B1′) may bind the complex of B1′ and TM1; and the second binding moiety on the reporter protein (B2′) may bind a second target molecule (TM2) and the binding moiety on the second regulator moiety (B2′') may bind the complex of B2′ and TM2, or the binding moiety on the second regulator moiety (B2″) may bind a second target molecule (TM2) and the second binding moiety on the reporter protein (B2′) may bind the complex of B2′ and TM2. Interaction of the binding moieties B1′ and B1″ may be dependent on the presence of a first target molecule TM1 and interaction of the binding moieties B2′ and B2″ may be dependent on the presence of a second target molecule TM2. The reporter protein may comprise further binding moieties (BX′; e.g., B3′, B4′ etc.). The reporter protein may comprise the same number of binding moieties as the number of heterologous amino acid sequences comprise in the reporter protein. Such further binding moieties will form an interacting pair with their respective binding moieties (e.g., BX′ will interact with BX″), wherein the respective binding moiety (BX″) will be comprised on a further regulator moiety.

The binding moieties on the reporter protein may be the same or different species of binding moiety (i.e., the reporter protein may comprise only one type of binding moiety, or may comprise two different types of binding moiety). The binding moieties on the regulator moieties may be the same or different species of binding moiety (i.e., each regulatory moiety may comprise the same (i.e., a single type of) binding moiety, or there may be a number of different species of regulatory moiety, where each species of regulatory moiety comprises a different binding moiety, preferably there are two different species of regulatory moieties). Each pair of binding moieties may interact with the same species of target molecule, such that one species (i.e., one type) of target molecule regulates the activity of the reporter protein. Alternatively, each pair of binding moieties may interact with a different species of target molecule, such that two or more species (i.e., types) of target molecules regulate the activity of the reporter protein. A species of target molecule may be considered to refer to a plurality of target molecules all of which are the same, i.e., the same molecule, compound or protein. Thus, the binding moieties on the reporter protein B1′ and B2′ may be the same species of binding moiety, in which case, typically the respective binding moieties on the regulator moieties B1″ and B2″ are the same species of binding moiety. In such cases, TM1 and TM2 are usually the same species of target molecule. Alternatively, the binding moieties on the reporter protein B1′ and B2′ may be different species of binding moiety, in which case, typically the respective binding moieties on the regulator moieties B1″ and B2″ are different species of binding moiety. In such cases, TM1 and TM2 are usually different species of target molecule.

The reporter protein may comprise a first heterologous amino acid sequence which is responsive to binding of a first regulator moiety and a second heterologous amino acid sequence which is responsive to binding of a second regulator moiety, wherein binding of the first regulator moiety to the first heterologous amino acid sequence and binding of the second regulator moiety to the second heterologous amino acid sequence, reversibly regulates activity of the reporter protein; wherein the reporter protein may further comprise a first binding moiety B1′ and a second binding moiety B2′. The first regulator moiety may further comprise a binding moiety B1″ and the second regulator moiety may further comprise a binding moiety B2″. The binding moiety B1″ of the first regulator moiety may be capable of interacting with the first binding moiety B1′ of the reporter protein and the binding moiety B2″ of the second regulator moiety may be capable of interacting with the second binding moiety B2′ on the enzyme. Preferably, interaction of the binding moieties B1′ and B1″ is dependent on the presence of a first target molecule TM1, and interaction of the binding moieties B2′ and B2″ is dependent on the presence of a second target molecule TM2. Thus, binding moieties B1′ and B1″ may form a pair of binding moieties that bind simultaneously to target molecule TM1, and binding moieties B2′ and B2″ may form a pair of binding moieties that bind simultaneously to target molecule TM2. Binding moieties B1′ and B1″ and binding moieties B2′ and B2″ may be the same pair of binding moieties or different pairs of binding moieties. The target molecules TM1 and TM2 may be the same species of target molecule or may be different species of target molecules.

In some instances, the reporter protein comprises a binding moiety B1′, which is capable of interacting with respective binding moiety B1″, typically through a target molecule TM1. The respective binding moiety B1″ may be linked (optionally through peptide linkers) to a first regulator moiety and a second regulator moiety, which are configured to bind to the first and second heterologous amino acid sequences on the reporter protein, respectively, to thereby activate the reporter protein in the presence of TM1. In some preferred instances, the reporter protein comprises two calmodulin protein inserts and a binding moiety B1′, wherein the respective binding moiety B1″ is linked (preferably through peptide linkers, optionally as a fusion protein) to two CaM-BPs. preferably CaM-BPs having a reduced binding affinity for calmodulin, as compared to a wild-type CaM-BP. The reporter protein may comprise two calmodulin protein inserts and a binding moiety B1′, wherein the respective binding moiety B1″ is linked (preferably through peptide linkers, optionally as a fusion protein) to two caged CaM-BPs, as further described herein, wherein each caged CaM-BP preferably comprises a CaM-BP linked to a calmodulin protein having a reduced binding affinity for the CaM-BP. as compared to a calmodulin protein inserts on the reporter protein.

As generally used herein a “binding moiety” or “binding moieties” refers to one or a plurality of molecules or biological or chemical components or entities that are capable of recognizing, interacting with and/or binding to each other, or preferably to one or more target molecules. The exact identity of the binding moieties used in the biosensors and reporter proteins described herein typically depends on the one or more target molecules to be detected by the reporter protein/biosensor. Thus, the binding moieties of the reporter protein/biosensor may be selected as those that bind to the target molecule(s) of interest. Binding moieties may be polypeptides, nucleic acids (e.g., single-stranded or double-stranded DNA or RNA), sugars, oligosaccharides, polysaccharides or other carbohydrates, lipids or any combinations of these such as glycoproteins, PNA constructs etc., or molecular components thereof. By way of example only, binding moieties may be, or comprise: (i) an amino acid sequence of a ligand binding domain of a receptor responsive to binding of a target molecule such as a cognate growth factor, a cytokine, a hormone (e.g., insulin), a neurotransmitter, etc.; (ii) an amino acid sequence of an ion or metabolite transporter capable of, or responsive to, binding of a target molecule such as an ion or metabolite (e.g., a Ca2+-binding protein, such as calmodulin or calcineurin, or a glucose transporter, or a solute binding protein); (iii) a zinc finger amino acid sequence responsive to zinc-dependent binding of a DNA target molecule; (iv) a helix-loop-helix amino acid sequence responsive to binding of a DNA target molecule; (v) a pleckstrin homology domain amino acid sequence responsive to binding of a phosphoinositide target molecule; (vi) an amino acid sequence of a Src homology 2- or Src homology 3-domain responsive to a signalling protein; (vii) an amino acid sequence of an antigen responsive to binding of an antibody target molecule; or (viii) an amino acid sequence of a protein kinase or phosphatase responsive to binding of a phosphorylatable or phosphorylated target molecule; (ix) ubiquitin-binding domains; (x) proteins or protein domains that bind small molecules, drugs or antibiotics such as rapamycin-binding FKBP and FRB domains; (xi) single- or double-stranded DNA, RNA or PNA constructs that bind nucleic acid target molecules, such as where the DNA or RNA are coupled or cross-linked to an amino acid sequence or other protein-nucleic acid interaction; and/or (xii) an affinity clamp such as a PDZ-FH3 domain fusion; inclusive of modified or engineered versions thereof. In some cases, one of the binding moieties may be a small molecule binding protein and the respective binding moiety may recognise (i.e., specifically bind) the “small molecule: binding moiety” complex, but not the small molecule or the binding moiety alone. For example, binding moiety B1′ or B2′ on the reporter protein may bind small molecule X, and binding moiety B1″ or B2″ may bind the complex of X: B1′ or X: B2′, respectively and vice versa. Preferably, the binding moieties are proteins. Preferably, the pairs of binding moieties are proteins that are capable of interacting with each other, either directly or preferably through binding to a target molecule.

Exemplary binding moieties, some which are described in the Examples, are set out in the following list. Thus, the binding moieties (e.g., B1′ and B1″ and/or B2′ and B2″) may be selected from the group consisting of: FKBP (SEQ ID NO: 36), FRB (SEQ ID NO: 37), calcineurin alpha and beta subunits (comprising SEQ ID NOs: 38 and 39, or comprising SEQ ID NO: 40), human serum albumin (HAS) GA binder (SEQ ID NO: 41), cyclophilin (SEQ ID NO: 42), antibody fragments, specifically α-amylase binding antibody VHH fragments, VHH1 (SEQ ID NO: 43) and VHH2 (SEQ ID NO: 44), HAS antibody VHH binder (SEQ ID NO: 45), a methothrexate binding antibody VHH fragment (SEQ ID NO: 46) or a nanoCLAMP for methotrexate binding (SEQ ID NO: 47); and variants thereof. The skilled person is readily capable of selecting a compatible pair of binding moieties that are capable of interacting, preferably though simultaneous binding to a target molecule. Variants are typically functional binding variants for the relevant respective binding moiety. The skilled person is able to generate antibodies and antibody (VHH) fragments targeting any number of known target molecules for use as binding moieties in the reporter protein/biosensor described herein. It will also be appreciated that binding moieties may be modified or chemically derivatised such as with binding agents such as biotin, avidin, epitope tags, lectins, carbohydrates or lipids. As will be readily appreciated by the skilled person, suitable binding moieties may be designed that bind to any target molecule of interest, for example by generating a VHH-single domain antibody (VHH1) that binds the target molecule (TMX), which may preferably be a peptide or a small molecule, and then generating a second VHH-single domain antibody (VHH2) to recognise the complex of ‘TMX:VHH1’. Generating such VHH-single domain antibodies having the required specificity to a known target is routine for the skilled person.

The binding moieties may be or comprise an antibody or antibody fragment, inclusive of monoclonal and polyclonal antibodies, recombinant antibodies, Fab and Fab′2 fragments, DARPins, diabodies, monobodies, nanoCLAMPs (CLostridal Antibody Mimetic Proteins, see e.g., SEQ ID NO: 47), and single chain antibody fragments (e.g. scVs). Suitably, the first and second binding moieties may be or comprise respective antibodies or antibody fragments (preferably scFv) that bind a target molecule.

In some instances, the binding moieties (e.g., B1′ and B1″ and/or B2′ and B2″) are, or comprise, amino acid sequences of an affinity clamp. The affinity clamp preferably comprises a recognition domain and, optionally, an enhancer domain. The recognition domain is typically capable of binding one or more target molecules, such as described in (i)-(ix) above. Recognition domains may include, but are not limited to, domains involved in phospho-tyrosine binding (e.g. SH2. PTB), phospho-serine binding (e.g. UIM, GAT, CUE, BTB/POZ, VHS, UBA, RING, HECT, WW, 14-3-3, Polo-box), phospho-threonine binding (e.g. FHA, WW, Polo-box), proline-rich region binding (e.g. EVH1, SH3, GYF), acetylated lysine binding (e.g. Bromo), methylated lysine binding (e.g. Chromo, PHD), apoptosis (e.g. BIR, TRAF, DED, Death, CARD, BH), cytoskeleton modulation (e.g. ADF, GEL, DH, CH, FH2), ubiquitin-binding domains or modified or engineered versions thereof, or other cellular functions (e.g. EH, CC, VHL, TUDOR, PUF Repeat, PAS, MH1, LRR1, IQ, HEAT, GRIP, TUBBY, SNARE, TPR, TIR, START, SOCS Box, SAM, RGS, PDZ, PB1. LIM, F-BOX, ENTH, EF-Hand, SHADOW, ARM, ANK). The enhancer domain typically increases or enhances the binding affinity for at least one or the target molecules. In some embodiments, the affinity may be increased by at least 10, 100 or 1000 fold compared to that of the recognition domain alone. The affinity clamp may further comprise a linker connecting the recognition domain and the enhancer domain.

In some instances, the affinity clamp comprises a recognition domain that comprises at least a portion or fragment of a PDZ domain and an enhancer domain that comprises at least a portion or fragment of a fibronectin type III domain. The PDZ domain may be derived from a human Erbin protein. Erbin-PDZ (ePDZ) binds to target molecules such as the C-termini of p120-related catenins (such as 8-catenin and Armadillo repeat gene deleted in Velo-cardio-facial syndrome (ARVCF)). Preferably, this instance of the affinity clamp further comprises the tenth (10th) type III (FN3) domain of human fibronectin as an enhancer domain. In some instances, the affinity clamp may comprise one or more connector amino acid sequences. For example, a connector amino acid sequence may connect the protease amino acid sequence (such as comprising a protease amino acid sequence) to the Erbin-PDZ domain, the Erbin-PDZ domain to the FN3domain and/or the FN3 domain to the inhibitor. Reference is also made to WO2009/062170, Zhuang & Liu, 2011, Comput. Theoret. Chem. 963 448, Huang et al, 2009, J. Mol. Biol. 392 1221, Huang et al., 2008, PNAS (USA) 105 6578, and Koide and Huang Methods Enzymol. 2013; 523:285-302 for a more detailed explanation of affinity clamp structure and function, and of particular affinity clamps that may be used in accordance with the invention. Preferably, an affinity clamp may comprise, or consist of, the sequence of SEQ ID NO: 76, or a variant or functional fragment thereof (e.g., as the recognition domain), and may bind to a RGS peptide ligand comprising, or consisting of, the sequence of SEQ ID NO: 82, or a variant thereof. Thus, binding moiety B1′ or B2′ may each comprise or consist of an affinity clamp, optionally comprising, or consisting of, the sequence of SEQ ID NO: 76, and the respective binding moieties B1″ or B2″ may each comprise or consist of a RGS peptide ligand, optionally comprising, or consisting of, the sequence of SEQ ID NO: 82, or vice versa.

The respective binding moieties in each pair of binding moieties (i.e., a binding moiety on the reporter protein (e.g., B1′ or B2′) and its respective binding moiety on a regulatory moiety (e.g., B1″ or B2″)) may be capable of directly interacting. Preferably, the binding moieties in each pair of binding moieties (i.e., a binding moiety of the reporter protein (e.g., B1′ or B2′) and its respective binding moiety on a regulatory moiety (e.g., B1″ or B2″)) may be capable of binding, interacting or forming a complex with a target molecule (e.g., TM1 or TM2). A target molecule may comprise a plurality of different subunits, domains, or epitopes, each of which may be bound by the respective binding moieties within a pair, to thereby co-localize the pair of binding moieties and hence the further components of the biosensors/reporter proteins described herein. Accordingly, the direct binding interaction between the target molecule and a pair of binding moieties suitably facilitates co-localization of the components of the biosensors described herein.

A binding moiety may be linked (i.e., conjugated, joined or fused) to the N- or C-terminus of the reporter protein, optionally through a linker. The reporter protein may comprise one binding moiety B1′, wherein binding moiety B1′ may be linked to the N-terminus or the C-terminus of the reporter protein, optionally through a linker. The reporter protein may comprise two binding moieties B1′ and B2′, wherein the first binding moiety (B1′) may be linked to the N-terminus of the reporter protein, optionally through a linker, and the second binding moiety (B2′) may be linked to the C-terminus of the reporter protein, optionally through a linker; or vice versa. The reporter protein and the one or more binding moieties (e.g., the first and second binding moieties B1′ and B2′) may preferably be provided as a fusion protein. The reporter protein and the one or more binding moieties may be provided as a single contiguous amino acid sequence. A single contiguous amino acid sequence may be provided comprising from N- to C-terminus, or from C- to N-terminus, the amino acid sequence of a binding moiety (B1′), optionally a linker, and the amino acid sequence of the reporter protein comprising the first and the second heterologous amino acid sequences. A single contiguous amino acid sequence may be provided comprising from N- to C-terminus the amino acid sequence of a first binding moiety (B1′), optionally a linker, the amino acid sequence of the reporter protein comprising the first and the second heterologous amino acid sequences, optionally a linker, and the amino acid sequence of a second binding moiety (B2′). The amino acid sequence of the first and second binding moieties may be the same or different. A binding moiety (e.g., B1′ and/or B2′) may be inserted within the amino acid sequence of the reporter protein, optionally the amino acid sequence of the binding moiety is flanked by linkers. Typically a binding moiety (e.g., B1′ and/or B2′) may be inserted within the amino acid sequence of the reporter protein at a location that does not influence or affect the activity of the reporter protein, for example a loop region. Suitable linkers are as described elsewhere herein.

A binding moiety (e.g., B1″ or B2′') may be linked (i.e., conjugated or fused) to a regulator moiety. A first binding moiety (B1″) may be linked to the first regulator moiety and a second binding moiety (B2″) may be linked to the second regulator moiety. Where the reporter protein comprises first and second heterologous amino acid sequences that are the same, and comprises a first binding moiety (B1′) and a second binding moiety (B2′) that are the same (B1′=B2′), a single species of regulator moiety may be linked to a binding moiety that is capable of interacting with both the first and second binding moieties. Where the reporter protein comprises one binding moiety (B1′), the respective binding moiety B1″ may be linked to both the first and second regulator moieties. Where the regulatory moiety is a protein or peptide, the binding moiety (e.g., B1″ or B2″) may be linked to the N- or C-terminus of the regulatory moiety, optionally through a linker. Each regulator moiety may comprise one binding moiety. A regulator moiety and a binding moiety may be provided as a fusion protein. A regulator moiety and a binding moiety may be provided as a single contiguous amino acid sequence. A single contiguous amino acid sequence may be provided comprising from N- to C-terminus: the amino acid sequence of a binding moiety, optionally a linker, and the amino acid sequence of a regulatory moiety; or from N-to C-terminus the amino acid sequence of a regulatory moiety, optionally a linker, and the amino acid sequence of a binding moiety (e.g., B1″-RM1, RM1-B1″, B2″-RM2, RM2-B2″, where “-” may comprise a linker). A binding moiety may be inserted within the amino acid sequence of a regulatory moiety, optionally wherein the amino acid sequence of the binding moiety may be flanked by linkers. Typically in such cases a binding moiety may be inserted within the amino acid sequence of the regulatory moiety at a location that does not influence or affect the binding of the regulatory moiety to its respective heterologous amino acid sequence. One binding moiety may comprise two regulator moieties. The binding moiety and two regulator moieties may be provided as a fusion protein. The binding moiety and two regulator moieties may be provided as a single contiguous amino acid sequence. A single contiguous amino acid sequence may be provided comprising from N- to C-terminus: a first regulator moiety, optionally a linker, a binding moiety, optionally a linker, and a second regulator moiety. Suitable linkers are as described elsewhere herein.

### Target Molecules

The reporter proteins/biosensors described herein may be capable of detecting one or more target molecules. For each target molecule to be detected, the biosensor typically comprises one or more pair(s) of binding moieties capable of binding (usually at the same time) to that target molecule, wherein for each pair of binding moieties, the reporter protein comprises one binding moiety and a regulatory moiety comprises the respective second binding moiety of the pair. The reporter proteins/biosensors may be capable of detecting one species or type of target molecule. The reporter proteins/biosensors may be capable of detecting two species or types of target molecule. The reporter protein may comprise a first binding moiety B1′ and a second binding moiety B2′ and there may be two species of regulator moiety, a first regulator moiety comprising a first binding moiety B1″ and a second regulator moiety comprising a second binding moiety B2″, wherein the binding moieties B1′ and B1″ form a binding pair and are capable of simultaneously interacting with a target molecule TM1, and the binding moieties B2′ and B2″ form a binding pair and are capable of simultaneously interacting with a target molecule TM2. Target molecules TM1 and TM2 may be the same species of target molecule or different species of target molecules. The reporter protein may comprise a first binding moiety B1′ and a second binding moiety B2′ that are the same (B1′=B2′) and there may be one species of regulator moiety comprising a binding moiety that forms a binding pair with either B1′ or B2′ in the presence of target molecule TM1 The reporter protein may comprise one binding moiety B1′ and the binding moiety B1″ that forms a binding pair with B1′ in the presence of target molecule TM1 is linked to a first and a second regulator moiety, wherein the first and second regulator moieties may be the same or different, depending on the heterologous amino acid sequences of the reporter protein.

The target molecule (e.g., TM1 and/or TM2) may be any ligand, analyte, ion (e.g., calcium, Ca²⁺), small organic molecule, epitope, domain, fragment, subunit, moiety or combination thereof. The target molecule (e.g., TM1 and/or TM2) may be a protein, for example including antibodies and antibody fragments, antigens, enzymes such as α-amylase, human, serum albumin, phosphoproteins, glycoproteins, lipoproteins and glycoproteins. The target molecule (e.g., TM1 and/or TM2) may be lipids, phospholipids, carbohydrates including simple sugars, disaccharides and polysaccharides; nucleic acids, nucleoprotein. The target molecule (e.g., TM1 and/or TM2) may be a small molecule, chemical entity or any other analyte, including drugs, such as immunosuppressive drugs including rapamycin (i.e., sirolimus), cyclosporine, and tacrolimus (i.e., FK506) and other pharmaceuticals including antibiotics, vitamins, banned substances, illicit drugs or drugs of addiction, chemotherapeutic agents and lead compounds in drug design and screening, molecules and analytes typically found in biological samples such as biomarkers, tumour and other antigens, receptors, DNA-binding proteins inclusive of transcription factors, hormones, neurotransmitters, growth factors, cytokines, receptors, metabolic enzymes, signalling molecules, nucleic acids such as DNA and RNA, membrane lipids and other cellular components, pathogen-derived molecules inclusive of viral, bacterial, protozoan, fungal and worm proteins, lipids, carbohydrates and nucleic acids. The target molecule may be a protease. This is particularly the case where the biosensor comprises a caged regulator moiety as described further herein. Typically, in such cases, in the presence of the target protease of the biosensor, the protease cleaves the target protease cleavage sites in the caged regulator moiety, thereby releasing the regulator moiety which then binds to the heterologous amino acid sequences of the reporter protein, thereby activating the reporter protein. In such cases where the target molecule is a protease and the biosensor comprises a caged regulator moiety, the reporter protein may not comprise a binding domain. In such cases where the target molecule is a protease and the biosensor comprises a caged regulator moiety, the caged regulator moiety may comprise a protease binding domain, which may act to increase the local concentration of the target protease at the protease cleavage sites of the caged regulator moiety.

Preferably, the target molecule (e.g., TM1 and/or TM2) may be selected from the group consisting of: (i) hormones, including: fertility hormones, such as Progesterone, Estradiol, luteinising hormone (LH) and follicle-stimulating hormone (FSH); stress hormones (e.g., hormones associated with psychological stress) such as cortisol and α-amylase; metabolic hormones (e.g., hormones associated with or providing an indication of metabolic state) such as insulin and glucose; (ii) therapeutic drugs such as methotrexate, Rapamycin, tacrolimus, and Cyclosporine A; and (iii) environmental contaminants, such as phenolic glucosides. The reporter protein/biosensor may be configured for detecting any one of said target molecules. The reporter protein/biosensor may be configured for detecting any two of said target molecules. In particularly preferred instances, the target molecules (e.g., TM1 and/or TM2) may be methotrexate, phenolic glucosides, Rapamycin, tacrolimus and/or Cyclosporine A. The reporter protein/biosensor may be configured for detecting any one of methotrexate, phenolic glucosides, Rapamycin, tacrolimus and/or Cyclosporine A. For example, TM1 and TM2 are the same and are selected from the group consisting of: methotrexate, phenolic glucosides, Rapamycin, tacrolimus and/or Cyclosporine A. The reporter protein/biosensor may be configured for detecting any two of methotrexate, phenolic glucosides, Rapamycin, tacrolimus and/or Cyclosporine A. For example, TM1 and TM2 are different and are each independently selected from the group consisting of: methotrexate, phenolic glucosides, Rapamycin, tacrolimus and/or Cyclosporine A. The reporter protein/biosensor may be configured for detecting rapamycin and Cyclosporine A. For example, TM1 is rapamycin and TM2 is Cyclosporine A. For example, TM1 is rapamycin and TM2 is rapamycin. For example, TM1 is Cyclosporine A and TM2 is Cyclosporine A. The skilled person would be readily capable of selecting suitable pairs of binding moieties, typically proteins or antibodies, which are capable of binding to the selected target molecule(s) of interest, for example using routine interaction screening and binding assays, such as for example surface plasmon resonance methods such as ELISA or Biacore.

Exemplary binding moieties and target molecules are described in the Examples. In some instances, the target molecule (e.g., TM1 and TM2) is an enzyme such as a amylase. In such instances, the first and second binding moieties may be antibodies therefor, such as exemplified camelid antibodies VHH1 and VHH2 (SEQ ID NOs: 43 and 44) or variants thereof (e.g., B1′ and B2′ each comprise VHH1 comprising the sequence SEQ ID NO: 43; and B1″ and B2″ each comprise VHH2 comprising the sequence SEQ ID NO: 44). In some instances, the target molecule (e.g., TM1 and TM2) is a small organic molecule such as rapamycin. In such instances, the first and second binding moieties may be, respectively FKBP and FRB (SEQ ID NOs: 36 and 37), or variants thereof (e.g., B1′ and B2′ each comprise FKBP comprising the sequence SEQ ID NO: 36; and B1″ and B2″ each comprise FRB comprising the sequence SEQ ID NO: 37). In some instances, the target molecule (e.g., TM1 and TM2) is a small organic molecule such as FK506 (i.e., tacrolimus). In such instances, the first and second binding moieties may be, respectively, an FKBP and a Calcineurin alpha/beta complex (SEQ ID NOs: 36, and 38 and 39 or 40), or variants thereof (e.g., B1′ and B2′ each comprise FKBP comprising the sequence SEQ ID NO: 36; and B1″ and B2″ each comprise Calcineurin alpha/beta complex comprising the sequences SEQ ID NOs: 38 and 39, or 40). In some instances, the target molecule (e.g., TM1 and TM2) is human serum albumin (HAS). In such instances, the first and second binding moieties may be, respectively, a HAS GA binder and a HAS-specific VHH (SEQ ID NOs: 41 and 45) or variants thereof (e.g., B1′ and B2′ each comprise a HAS GA binder comprising the sequence SEQ ID NO: 41; and B1″ and B2″ each comprise a HAS-specific VHH comprising the sequence SEQ ID NO: 45). In some instances, the target molecule is cyclosporin (e.g., TM1 and TM2). In such instances, the first and second binding moieties may be, respectively, cyclophilin (SEQ ID NO: 42) and a Calcineurin alpha/beta complex (SEQ ID NOs: 38 and 39, or 40), or variants thereof (e.g., B1′ and B2′ each comprise a cyclophilin comprising the sequence SEQ ID NO: 38; and B1″ and B2″ each comprise a Calcineurin alpha/beta complex comprising the sequences of SEQ ID NOs: 38 and 39, or 40). In some instances, the target molecule (e.g., TM1 and TM2) is methotrexate (MTX). In such instances, the first and second binding moieties may be, respectively, a MTX-specific VHH and a MTX-specific nanoCLAMP (SEQ ID NOs: 46 and 47) or variants thereof (e.g., B1′ and B2′ each comprise a MTX-specific VHH comprising the sequence SEQ ID NO: 46; and B1″ and B2″ each comprise a MTX-specific nanoCLAMP comprising the sequence SEQ ID NO: 47). In some instances, the target molecules are rapamycin and cyclosporin (e.g., TM1 is rapamycin and TM2 is cyclosporin). In such instances, binding moiety B1′ may comprise FKBP (SEQ ID NO: 36) and binding moiety B2′ may comprise cyclophilin (SEQ ID NO: 42), and binding moiety B1″ may comprise FRB (SEQ ID NO: 37) and binding moiety B2″ may comprise a Calcineurin alpha/beta complex (SEQ ID NOs: 38 and 39, or 40).

### Biosensors

The reporter proteins described herein are designed to be incorporated into biosensors, for example for detection of target molecules. Described herein are biosensors comprising the reporter proteins as described herein. The biosensors typically comprise: (i) a reporter protein as described herein; and (ii) one or more regulatory moieties as described herein. The biosensor preferably comprises: (i) a reporter protein as described herein; (ii) a first regulator moiety as described herein; and (iii) a second regulator moiety as described herein. The biosensor may comprise: (i) a reporter protein as described herein; and (ii) one or more caged regulatory moieties as described herein. Where the reporter protein is an enzyme, the biosensor may further comprise a substrate for the enzyme (i.e., an enzyme substrate or substrate molecule), as further described herein.

The functioning of the reporter proteins/biosensors described herein is based on the principle that the reporter proteins may have an array of conformations in which it is inactive. While the conformation of the active reporter protein is likely to be similar to that of the wild type parental molecule, there may be a number of different conformations where the reporter protein is inactive. Thus, a number of conformation-induced deactivating changes in the reporter protein may be induced by two or more insertions of heterologous amino acid sequences at different insertion locations. Conformational changes at each of these insertion sites may then epistatically interact or synergistically combine to convert the reporter protein from an inactive to an active state.

Thus, typically binding of a regulator moiety to a heterologous amino acid sequence induces a conformational change in the heterologous amino acid sequence, which in turn induces a conformational change in the reporter protein. Binding of a regulatory moiety at each of the heterologous amino acid sequences comprised in the reporter protein (e.g., binding of the first regulator moiety to the first heterologous amino acid sequence and binding of the second regulator moiety to the second heterologous amino acid sequence) induces a number of conformational changes that sum to induce a conformational change in the reporter protein that converts it from an inactive state to an active state. Thus, typically, only binding of regulatory moieties at all of the heterologous amino acid sequences comprised in the reporter protein will activate the reporter protein. Hence the reporter proteins/biosensors of the invention may be characterised as “AND-gate” biosensors since two concurrent inputs (e.g., the first and second regulator moieties binding the first and second heterologous amino acid sequences, respectively) are required to trigger the output, i.e., the activity of the reporter protein producing a detectable and/or measurable signal.

The activity of the reporter protein may ultimately be dependent on the presence of one or more target molecules (e.g., TM1, or TM1 and TM2). Typically, the reporter proteins/biosensors of the invention are designed for detection of one or two target molecules (e.g., TM1=TM2, or TM1 and TM2). Typically the reporter protein comprises one half of a pair of binding moieties (e.g., B1′ or B2′), with the respective other half of the pair of binding moieties (e.g., B1″ or B2″) being comprised on the regulatory moieties. Typically, the pair of binding moieties (e.g., B1′ and B1″, or B2′ and B2″) interact through mutual recognition of a target molecule (e.g., TM1 or TM2, respectively). Thus, in the presence of a target molecule the pair of binding moieties simultaneously bind the target molecule and thereby co-localise the regulatory moiety with the reporter protein. This enhances or facilitates binding of the regulatory moiety to a heterologous amino acid sequence comprised in the reporter protein. There may be two pairs of binding moieties within the biosensors, such that the reporter protein comprises two binding moieties, each forming one half of two pairs of binding moieties, where the respective other halves of the pairs are located on the regulatory moieties.

For the detection of one target molecule, the two pairs of binding moieties may be the same, such that in the presence of the target molecule both pairs of binding moieties concurrently bind respective target molecules to co-localise the reporter protein with two regulatory moieties. Alternatively, for the detection of one target molecule, the reporter protein may comprise one binding moiety and the respective second binding moiety may be linked to two regulator moieties, such that in the presence of the target molecule the single pair of binding moieties bind the target molecule to co-localise the reporter protein with two regulatory moieties. In either case, this enhances and facilitates binding of a regulator moiety to each of the heterologous amino acid sequence comprised in the reporter protein, thus two sets of conformational changes are induced in the reporter protein thereby converting it from an inactive state to an active state. The requirement for two separate inputs results in low background noise (e.g., since random activation of the reporter protein is highly unlikely), and a high dynamic range of the reporter protein. It appears also to favour higher maximal catalytic activity of the biosensor compared to the single insertion variants. Typically, there is a trade-off between the dynamic range and catalytic activity. It has been surprisingly found that using two high catalytic activity and low dynamic range modules allows for circumventing this trade-off, to generate modules with both a high catalytic activity and high dynamic range. This is exemplified by the biosensors described herein, such as the BLA-2CaM MTX biosensor. Further pairs of binding moieties, and further heterologous amino acid sequences may be included to require further inputs, which may further enhance the dynamic range of the reporter protein.

For the detection of two target molecules, the two pairs of binding moieties may be different, such that in the presence of only one of the target molecules, only one pair of binding moieties concurrently bind the target molecule to co-localise the reporter protein with only a single regulatory moiety. The presence of a second target molecule is required for activation of the reporter protein, such that in the presence of the first and second target molecules both pairs of binding moieties concurrently bind their respective target molecules to co-localise the reporter protein with two regulatory moieties. The regulatory moieties bind to each of the heterologous amino acid sequences comprised in the reporter protein, thus inducing two sets of conformational changes in the reporter protein and thereby converting it from an inactive state to an active state. Thus, the requirement for two separate inputs may be used to detect the simultaneous presence of two target molecules.

Thus, in some instances, in the presence of a first target molecule (TM1), a first binding moiety on the reporter protein (B1′) interacts with its respective binding moiety (B1″) on a first regulator moiety; and in the presence of a second target molecule (TM2), a second binding moiety on the reporter protein (B2′) interacts with its respective binding moiety (B2″) on the second regulator moiety, which co-localises the reporter protein with the first and second regulator moieties and facilitates binding of the first regulator moiety to a first heterologous amino acid sequence comprised in the reporter protein and binding of the second regulator moiety to a second heterologous amino acid sequence comprised in the reporter protein. Binding of the first regulator moiety to the first heterologous amino acid sequence and binding of the second regulator moiety to the second heterologous amino acid sequence (typically concurrently, simultaneously) typically induces a conformational change in the reporter protein and thereby reversibly regulates activity of the reporter protein. Thus, ultimately, the presence of a first target molecule TM1 and a second target molecule TM2 reversibly regulates activity of the reporter protein.

The activity of the reporter protein may then be detected by suitable methods depending on the identity of the reporter protein, to provide a read-out of the presence of the one or more target molecules.

### Compositions, Kits and Detection Devices

As described above, provided herein are biosensors comprising (i) a reporter protein as described herein; (ii) a first regulator moiety as described herein; and (iii) a second regulator moiety as described herein. Also described herein are compositions or kits comprising: (a) a reporter protein as described herein; or (b) a biosensor as described herein. Where the reporter protein is an enzyme, the composition or kit may further comprise a substrate for the enzyme, as further described herein.

The compositions or kits may further comprise one or more target molecules. For example, the composition or kit may further comprise a first target molecule (TM1) and/or a second target molecule (TM2). Suitable target molecules are further described herein, but may preferably be selected from methotrexate, phenolic glucosides, Rapamycin,, tacrolimus, Cyclosporine A. The compositions or kits may further comprise a first species of target molecule (e.g., TM1=TM2, where they are the same), for example for use as a positive control. The compositions or kits may further comprise two species of target molecules (i.e., TM1 and TM2, where they are different), for example for use a positive control.

The compositions or kits may further comprise a sample, typically the sample is provided by the user and added to the composition or kit as provided. The sample may be suspected of containing the one or more target molecules, or may be being tested for the presence of the one or more target molecules. Thus, the sample may or may not comprise target molecule TM1 and/or target molecule TM2, or it may not be known whether the sample comprises TM1 and/or TM2. The sample may be a biological or environmental sample, preferably a biological sample. A biological sample may be understood to mean a sample derived from organic matter, preferably a sample obtained from a living organism. The biological sample may be obtained from animals (preferably mammals, most preferably humans), plants or fungi. The sample may be obtained from agriculturally important animals or plants, such as livestock (e.g., cows, sheep, pigs, chickens, etc.) or crops (e.g., vegetables, grains, or other foodstuffs). The sample may be a biological sample, wherein the biological sample is selected from: tissue, blood, plasma, serum, saliva, interstitial fluid, milk, juice, sap, or homogenate. An environmental sample may be understood to mean a sample derived from inorganic matter, such as a water sample, rock sample or air sample. The skilled person would be readily capable of preparing a particular sample for analysis using the compositions and kits and/or according to the methods described herein.

The compositions or kits comprising the reporter proteins or biosensors of the invention may further comprise a second enzyme, also referred to herein as an adaptor enzyme. The terms “second enzyme” and “adaptor enzyme” may be used interchangeably. In instances where the reporter protein is an enzyme, the compositions or kits may further comprise an adaptor enzyme (i.e., a second enzyme). The role of the adaptor enzyme is typically to convert the product of the enzyme of the biosensor into a read-out that is easier or simpler to detect. Thus, typically the product of the enzyme of the biosensor is the substrate of the adapter enzyme. For example, the enzyme of the biosensor may be trehalase, which catalyses the conversion of trehalose into glucose. The adapter enzyme may then be for example a GDH enzyme that converts glucose into gluconolactone and electrons. The electrons produced by the GDH enzyme may then be readily measured.

In some instances, the adapter enzyme may comprise a heterologous amino acid sequence which is responsive to a peptide, wherein binding of the peptide to the heterologous amino acid sequence reversibly regulates catalytic activity of the second enzyme, wherein the substrate of the adapter enzyme is the catalytic product of the reporter protein as described herein where the reporter protein is an enzyme. The adapter enzyme is typically a fast enzyme, preferably a diffusion limited enzyme. The adapter enzyme may be an oxidoreductase, preferably a GDH, most preferably PQQ-GDH as described herein. The adapter enzyme may be a β-lactamase, as described herein. The adapter enzyme may be a carbonic anhydrase, as described herein, wherein the read-out of enzyme activity and hence the biosensor may be a change in pH. The heterologous amino acid sequence is typically provided as an insert within the amino acid sequence of the adapter enzyme. The heterologous amino acid sequence may be a calmodulin protein, or functional fragment thereof, as described herein. The peptide may be a calmodulin-binding peptide (CaM-BP), as described herein. Thus, the compositions or the kits may further comprise a GDH enzyme comprising a calmodulin protein, or functional fragment thereof, which is responsive to a calmodulin-binding peptide (CaM-BP), wherein binding of the CaM-BP to the calmodulin protein, or functional fragment thereof, reversibly activates the catalytic activity of the GDH enzyme, optionally wherein the catalytic product of the reporter protein as described herein is a substrate of the GDH enzyme. The calmodulin protein, or functional fragment thereof, may be inserted in the sequence of the GDH enzyme, preferably in the loop connecting beta-sheets 5 and 6, most preferably in a location corresponding to positions 403-405 (amino acid residues Ser403 to Asn405) of PQQ-GDH of SEQ ID NO: 5. The adapter protein comprising a heterologous amino acid sequence may comprise or consist essentially of the sequence of SEQ ID NO: 63.

In some instances the adapter enzyme comprising a heterologous amino acid sequence which is responsive to a peptide may further comprise a binding moiety (AB′), and the peptide may further comprise a respective binding moiety (AB″). The binding moieties (AB′ and/or AB″) may be as described herein. The two binding moieties typically form a pair of binding moieties, as described herein. The pair of binding moieties (AB′ and AB″) typically interact, preferably through a target molecule (TM_{AB}). Thus, the presence of the target molecule (TM_{AB}) causes the binding moieties (AB′ and AB″) to interact, thereby co-localising the peptide with the adapter enzyme, the peptide then binds to the heterologous amino acid sequence and activates the catalytic activity of the adapter enzyme. Thus, activation of the catalytic activity of the adapter enzyme may be dependent on the presence of a target molecule (TM_{AB}), which may be as described herein. Preferably, the target molecule of the adapter enzyme (TM_{AB}) is different to the target molecules of the reporter proteins (TM1 and TM2) described herein included in the biosensors of the invention or the compositions or kits of the invention. Accordingly, the pair of binding moieties associated with the adapter enzyme (AB′ and AB″) is typically different from the pairs of binding moieties associated with the reporter enzyme as described herein (B1′ and B1″, and B2′ and B2″). Thus, another input to the biosensor system may be added by including an adapter enzyme. For example, a signal from the adapter enzyme may only be detected (i) in the presence of the one or more target molecules (TM1 and TM2) of the reporter protein/biosensors of the invention as described herein, and (ii) in the presence of the target molecule (TM_{AB}) of the adapter enzyme. Where the reporter protein is an enzyme, activation of the activity of the reporter protein is required to produce the product of the reporter protein, which product of the reporter protein is typically the substrate of the adapter enzyme.

Also described herein is a detection device that comprises a cell or chamber that comprises a reporter protein, a biosensor, or a composition, as described herein. Suitably, a sample may be introduced into the cell or chamber to thereby facilitate detection of the one or more target molecules (e.g., TM1 and TM2). The sample may be as described herein, e.g., a biological or an environmental sample. In certain instances, the detection device is capable of providing an electrochemical, acoustic and/or optical signal that indicates the presence of the one or more target molecules (e.g., TM1 and TM2). In some instances, the detection device may comprise an electrode. In some instances the detection device may comprise a semiconductor device. In some instances the detection device is a device adapted for amperometry. The device may comprise screen printed electrodes, preferably layered with a dry mixture comprising the biosensor of the invention and preferably an electron mediator.

The detection device may further provide a disease diagnosis from a diagnostic target result by comprising: a processor and a memory coupled to the processor, the memory including computer readable program code components that, when executed by the processor, perform a set of functions including analysing a diagnostic test result and providing a diagnosis of the disease or condition.

The detection device may further provide for communicating a diagnostic test result by comprising: a processor and a memory coupled to the processor, the memory including computer readable program code components that, when executed by the processor, perform a set of functions including: transmitting a diagnostic result to a receiving device; and optionally receiving a diagnosis of the disease or condition from the or another receiving device.

### Preferred Biosensors

As will be appreciated any of the above-described components of the reporter proteins/biosensors may be combined together to form a reporter protein/biosensor of the invention. In this section we describe some of the most preferred reporter protein/biosensors architectures, which are non-limiting, but are provided to further illustrate the present invention and the functioning of the reporter proteins/biosensors described herein.

Thus, also described herein is a reporter protein, which is an enzyme or fluorescent protein, comprising a first calmodulin protein sequence which is responsive to binding of a first CaM-BP, and a second calmodulin protein sequence which is responsive to binding of a second CaM-BP, wherein binding of the first CaM-BP to the first calmodulin protein sequence and binding of the second CaM-BP to the second calmodulin protein sequence, reversibly regulates the activity of the reporter protein, wherein the first calmodulin protein sequence and the second calmodulin protein sequence are provided as inserts within the amino acid sequence of the reporter protein, preferably inserted at different locations within the amino acid sequence of the reporter protein. Preferably wherein the reporter protein is an enzyme.

Also described herein is a reporter protein, which is an enzyme or fluorescent protein, comprising a first calmodulin protein sequence which is responsive to binding of a first CaM-BP, and a second calmodulin protein sequence which is responsive to binding of a second CaM-BP, wherein binding of the first CaM-BP to the first calmodulin protein sequence and binding of the second CaM-BP to the second calmodulin protein sequence, reversibly regulates the activity of the reporter protein, wherein the first and second calmodulin protein sequences are provided as inserts within the amino acid sequence of the reporter protein, wherein the reporter protein further comprises two binding moieties (B1′ and B2′). Preferably wherein the binding moieties B1′ and B2′ are both proteins. The binding moieties B1′ and B2″ may bind the same species of target molecule (TM1=TM2). The amino acid sequence of the binding moieties B1′ and B2′ may be the same. The first and second CaM-BPs may each further comprise a respective binding moiety (B1″ and B2″, wherein B1″=B2″) that forms a binding pair with the binding moieties on the reporter protein, such that the binding moiety on the CaM-BP (B1″ and B2″) may be capable of interacting with the binding moieties on the reporter protein (B1′ and B2′) through a single species of target molecule (TM1=TM2). Alternatively, the binding moieties B1′ and B2″ may bind different species of target molecule (TM1 and TM2, respectively). The amino acid sequence of the binding moieties B1′ and B2′ may be the different. The first CaM-BP may further comprise a binding moiety (B1″) that forms a binding pair with the binding moiety on the reporter protein (B1′) and the second CaM-BP may further comprise a respective binding moiety (B2″) that forms a binding pair with the binding moiety on the reporter protein (B2′), such that the two pairs of binding moieties interact through two different species of target molecules: B1′ and B1″ interact through TM1, and B2′ and B2″ interact through TM2.

Also described herein is a reporter protein, which is an enzyme or fluorescent protein, comprising a first calmodulin protein sequence which is responsive to binding of a first CaM-BP, and a second calmodulin protein sequence which is responsive to binding of a second CaM-BP, wherein binding of the first CaM-BP to the first calmodulin protein sequence and binding of the second CaM-BP to the second calmodulin protein sequence, reversibly regulates the activity of the reporter protein, wherein the first and second calmodulin protein sequences are provided as inserts within the amino acid sequence of the reporter protein, wherein the reporter protein further comprises a binding moiety (B1′). Preferably, the binding moiety B1′ is a protein. The first and second CaM-BPs are linked (preferably, the first and second CaM-BPs are comprised within the same contiguous amino acid sequence), and further comprise a respective binding moiety (B1″) (preferably also further comprised within the same contiguous amino acid sequence) that forms a binding pair with the binding moiety (B1′) on the reporter protein, such that the binding moiety on the CaM-BPs (B1″) may be capable of interacting with the binding moiety on the reporter protein (B1′) through a single species of target molecule (TM1). Also descriebd herein is a biosensor comprising a first component and a second component, wherein the first component comprises a reporter protein, which is an enzyme or fluorescent protein, comprising a first calmodulin protein sequence which is responsive to binding of a first CaM-BP, and a second calmodulin protein sequence which is responsive to binding of a second CaM-BP, wherein binding of the first CaM-BP to the first calmodulin protein sequence and binding of the second CaM-BP to the second calmodulin protein sequence, reversibly regulates the activity of the reporter protein, wherein the first and second calmodulin protein sequences are provided as inserts within the amino acid sequence of the reporter protein, wherein the reporter protein further comprises a binding moiety (B1′); and the second component comprises a binding moiety (B1″) that is capable of interacting with the binding moiety (B1′) on the reporter protein, which is linked to the first CaM-BP and the second CaM-BP. Optionally, the first and second CaM-BPs have a reduced affinity for calmodulin as compared to a wild-type CaM-BP. Alternatively, the CaM-BPs are caged CaM-BP, i.e., are each further linked to a calmodulin protein having a reduced binding affinity for the CaM-BP, as compared to the binding affinity of the calmodulin insert on the reporter protein for the CaM-BP.

Thus, in preferred instances, (i) the heterologous amino acid sequences (e.g., the first and second, and any further heterologous amino acid sequences) are each a calmodulin protein or a variant or functional fragment thereof, (ii) the regulator moiety (e.g., the first and second regulator moiety) is a peptide, preferably a CaM-BP. (iii) the reporter protein is an enzyme or fluorescent protein, (iv) the reporter protein comprises two heterologous amino acid sequences. (v) the reporter protein comprises two binding domains (e.g., a first and a second binding moiety B1′ and B2′), and (vi) the reporter protein/biosensor targets one or two different target molecules, depending on whether the two pairs of binding moieties are the same or different.

In some instances the reporter protein comprising a first heterologous amino acid sequence and a second heterologous amino acid sequence, comprises or consists of a sequence having at least 60%, 70%, 80%, 85%, 90%, 95%, 96%, 97%, 98%, 99% or at least 100% sequence identity to. optionally at least 80%, preferably at least 90%, most preferably at least 95%, sequence identity to any one of SEQ ID NOs: 51, 52, 57, 62, 75, 81, 86 or 89. In some instances the reporter protein comprising a first heterologous amino acid sequence and a second heterologous amino acid sequence comprises or consists of a sequence of any one of SEQ ID NOs: 51, 52, 57, 62, 75, 81, 86 or 89, or a variant thereof. In some instances the reporter protein comprising a first heterologous amino acid sequence and a second heterologous amino acid sequence comprises or consists of a sequence of any one of SEQ ID NOs: 51, 52, 57, 62, 75, 86 or 89, wherein one or both of the calmodulin inserts are replaced with a different heterologous amino acid sequences as described herein, such as for example an affinity clamp or SBP. In some instances the reporter protein comprising a first heterologous amino acid sequence and a second heterologous amino acid sequence comprises or consists of a sequence of SEQ ID NO: 81, wherein one or both of the affinity clamp inserts are replaced with a different heterologous amino acid sequence as described herein, such as for example a calmodulin protein or SBP.

Preferred biosensors typically comprise the preferred reporter proteins as described above. The biosensor may be configured for detection of rapamycin, and the biosensor may comprise (i) a reporter protein (which may be an enzyme or a fluorescent protein, optionally a trehalase enzyme, a GDH enzyme or a β-lactamase enzyme), comprising two calmodulin protein inserts at different locations within the reporter protein amino acid sequence, and two FKBP binding moieties, one at the N-terminus and one at the C-terminus of the reporter protein amino acid sequence; and (ii) a regulatory moiety which comprises a calmodulin binding peptide and a FRB binding moiety. Where the reporter protein is an enzyme the biosensor may further comprise the substrate of the enzyme. Where the reporter protein is trehalase the biosensor may further comprise the enzyme substrate trehalose. Where the reporter protein is β-lactamase the biosensor may further comprise the enzyme substrate nitrocefin. Thus, a biosensor for detection of rapamycin may comprise a first component comprising or consisting of a sequence having at least 60%, 70%, 80%, 85%, 90%, 95%, 96%, 97%, 98%, 99% or 100%, preferably 80%, most preferably 90% sequence identity to SEQ ID NO: 53 or 58; and a second component comprising or consisting of a sequence having at least 60%, 70%, 80%, 85%, 90%, 95%, 96%, 97%, 98%, 99% or 100%, preferably at least 80%. most preferably at least 90%, sequence identity to SEQ ID NO: 65.

The biosensor may be configured for detection of tacrolimus, and the biosensor may comprise (i) a reporter protein (which may be an enzyme or a fluorescent protein, optionally a trehalase enzyme, a GDH enzyme or a β-lactamase enzyme), comprising two calmodulin protein inserts at different locations within the reporter protein amino acid sequence, and two FKBP binding moieties, one at the N-terminus and one at the C-terminus of the reporter protein amino acid sequence; and (ii) a regulatory moiety which comprises a calmodulin binding peptide and a calcineurin A and B (CalA/B) binding moiety. Where the reporter protein is an enzyme the biosensor may further comprise the substrate of the enzyme. Thus, a biosensor for detection of tacrolimus may comprise a first component comprising or consisting of a sequence having at least 60%, 70%, 80%, 85%, 90%, 95%, 96%, 97%, 98%, 99% or 100%, preferably 80%, most preferably 90% sequence identity to SEQ ID NO: 53 or 58; and a second component comprising or consisting of a sequence having at least 60%, 70%, 80%, 85%, 90%, 95%, 96%, 97%, 98%, 99% or 100%, preferably at least 80%, most preferably at least 90%, sequence identity to SEQ ID NO: 66.

The biosensor may be configured for detection of methotrexate (MTX), and the biosensor may comprise (i) a reporter protein (which may be an enzyme or a fluorescent protein, optionally a trehalase enzyme, a GDH enzyme or a β-lactamase enzyme), comprising two calmodulin protein inserts at different locations within the reporter protein amino acid sequence, and two MTX-specific VHH binding moieties, one at the N-terminus and one at the C-terminus of the reporter protein amino acid sequence; and (ii) a regulatory moiety which comprises a calmodulin binding peptide and a MTX-specific nanoCLAMP binding moiety. Where the reporter protein is an enzyme the biosensor may further comprise the substrate of the enzyme. Thus, a biosensor for detection of MTX may comprise a first component comprising or consisting of a sequence having at least 60%, 70%, 80%, 85%, 90%, 95%, 96%, 97%, 98%, 99% or 100%, preferably 80%, most preferably 90% sequence identity to any one of SEQ ID NO: 59, 84 or 87; and a second component comprising or consisting of a sequence having at least 60%, 70%, 80%, 85%, 90%, 95%, 96%, 97%, 98%, 99% or 100%, preferably at least 80%, most preferably at least 90%, sequence identity to SEQ ID NO: 67 or 88.

The biosensor may be configured for detection of rapamycin and cyclosporin, and the biosensor may comprise (i) a reporter protein (which may be an enzyme or a fluorescent protein, optionally a trehalase enzyme, a GDH enzyme or a β-lactamase enzyme) comprising two calmodulin protein inserts at different locations within the reporter protein amino acid sequence; a FKBP binding moiety, optionally at the N-terminus of the reporter protein amino acid sequence; and a Cyclophilin binding moiety, optionally at the C-terminus of the reporter protein amino acid sequence; (ii) a regulatory moiety which comprises a calmodulin binding peptide and a FRB binding moiety; and (iii) a regulatory moiety which comprises a calmodulin binding peptide and a calcineurin A and B (CalA/B) binding moiety. Where the reporter protein is an enzyme the biosensor may further comprise the substrate of the enzyme. Where the reporter protein is trehalase the biosensor may further comprise the enzyme substrate trehalose. Thus, a biosensor for detection of rapamycin may comprise a first component comprising or consisting of a sequence having at least 60%, 70%, 80%, 85%, 90%, 95%, 96%, 97%, 98%, 99% or 100%, preferably 80%, most preferably 90% sequence identity to SEQ ID NO: 54; a second component comprising or consisting of a sequence having at least 60%, 70%, 80%, 85%, 90%, 95%, 96%, 97%, 98%, 99% or 100%, preferably at least 80%, most preferably at least 90%, sequence identity to SEQ ID NO: 65;and a third component comprising or consisting of a sequence having at least 60%, 70%, 80%, 85%, 90%, 95%, 96%, 97%, 98%, 99% or 100%, preferably at least 80%, most preferably at least 90%, sequence identity to SEQ ID NO: 66.

The biosensor may be configured for detection of a protease, and the biosensor may comprise (i) a reporter protein (which may be an enzyme or a fluorescent protein, optionally a trehalase enzyme, a GDH enzyme or a β-lactamase enzyme), comprising two calmodulin protein inserts at different locations within the reporter protein amino acid sequence; and (ii) a regulatory moiety which comprises a CaM-BP linked to a calmodulin protein having a reduced binding affinity for the CaM-BP, as compared to the binding affinity of the calmodulin protein inserts, through a peptide linker comprising a cleavage site for the target protease. Where the reporter protein is an enzyme the biosensor may further comprise the substrate of the enzyme.

The biosensor may be configured for detection of a target molecule (TM1), and the biosensor may comprise (1) a reporter protein (which may be an enzyme or a fluorescent protein, optionally a trehalase enzyme, a GDH enzyme or a β-lactamase enzyme), comprising two calmodulin protein inserts at different locations within the reporter protein amino acid sequence, and a first binding moiety (B′) specific for TM1, optionally at the N- or C-terminus of the reporter protein amino acid sequence; and either (2-i) a regulatory moiety which comprises a second binding moiety (B″) also specific for TM1 or specific for the complex of TM1 and B′, linked to two CaM-BPs, optionally wherein each CaM-BP has a reduced binding affinity for calmodulin as compared to a wild-type CaM-BP, or (2-ii) a regulatory moiety which comprises a second binding moiety (B″) also specific for TM1 or specific for the complex of TM1 and B′, linked to two caged CaM-BPs, wherein each caged CaM-BP comprises a CaM-BP linked to a calmodulin protein having a reduced binding affinity for the CaM-BP, as compared to the binding affinity of the calmodulin protein inserts of the reporter protein. Where the reporter protein is an enzyme the biosensor may further comprise the substrate of the enzyme.

### Cascades

Two or more biosensors described herein may be used in tandem to create cascades, for example enzymatic cascades, of which multiple steps may be regulated. Two or more biosensors may be used at directly sequential steps in a cascade, or at indirectly sequential steps (for example wherein one or more intervening steps are not monitored by a biosensor).

For example, a first biosensor as described herein may be used to convert trehalose to glucose, and a second biosensor may be used to convert glucose to gluconolactate. The first biosensor may be any biosensor comprising trehalase. Such a first biosensor may be configured to respond to rapamycin, for example the first biosensor may comprise a reporting protein comprising or consisting of a sequence having at least 60%, 70%, 80%, 85%, 90%, 95%, 96%, 97%, 98%, 99% or at least 100% sequence identity to, optionally at least 80%, preferably at least 90%, most preferably at least 95%, sequence identity to SEQ ID NO: 54, optionally also comprising a first heterologous amino acid sequence comprising or consisting of a sequence having at least 60%, 70%, 80%, 85%, 90%, 95%, 96%, 97%, 98%, 99% or at least 100% sequence identity to, optionally at least 80%, preferably at least 90%, most preferably at least 95%, sequence identity to SEQ ID NO: 65, and a second heterologous amino acid sequence comprising or consisting of a sequence having at least 60%, 70%, 80%, 85%, 90%, 95%, 96%, 97%, 98%, 99% or at least 100% sequence identity to, optionally at least 80%, preferably at least 90%, most preferably at least 95%, sequence identity to SEQ ID NO: 66.

The second biosensor may be any biosensor comprising GDH. Such a second biosensor may be configured to respond to cyclosporine A, for example the second biosensor may comprise a reporting protein comprising or consisting of a sequence having at least 60%, 70%, 80%, 85%, 90%, 95%, 96%, 97%, 98%, 99% or at least 100% sequence identity to, optionally at least 80%, preferably at least 90%, most preferably at least 95%, sequence identity to SEQ ID NO: 64, optionally also comprising a first heterologous amino acid sequence and a second heterologous amino acid sequence comprising or consisting of a sequence having at least 60%, 70%, 80%, 85%, 90%, 95%, 96%, 97%, 98%, 99% or at least 100% sequence identity to, optionally at least 80%, preferably at least 90%, most preferably at least 95%, sequence identity to SEQ ID NO: 66. Alternatively, such a second biosensor may be configured to respond to methotrexate (MTX), for example the second biosensor may comprise a reporting protein comprising or consisting of a sequence having at least 60%, 70%, 80%, 85%, 90%, 95%, 96%, 97%, 98%, 99% or at least 100% sequence identity to, optionally at least 80%, preferably at least 90%, most preferably at least 95%, sequence identity to SEQ ID NO: 84, optionally also comprising a first heterologous amino acid sequence and a second heterologous amino acid sequence comprising or consisting of a sequence having at least 60%, 70%, 80%, 85%, 90%, 95%, 96%, 97%, 98%, 99% or at least 100% sequence identity to, optionally at least 80%, preferably at least 90%, most preferably at least 95%, sequence identity to SEQ ID NO: 67 or 88.

The skilled person will appreciate that any biosensors described herein may be configured such that they form a cascade.

### Methods

Also described herein is a method of detecting one or more target molecules (preferably, one target molecule or two target molecules) (e.g., TM1 and TM2), said method including a step of contacting one or more of the biosensors described herein with a sample under conditions suitable for detection of the presence or absence of the one or more target molecule in the sample. Also described herein is a method of detecting one or more target molecules (preferably, one target molecule or two target molecules) (e.g., TM1 and TM2), said method including the step of contacting one or more of the reporter proteins described herein with a sample under conditions suitable for detection of the presence or absence of the one or more target molecules in the sample.

The sample may be any sample suspected of containing the one or more target molecules. The sample may or may not comprise the one or more target molecules (e.g., target molecule TM1 and/or target molecule TM2 as described herein). The sample may be a biological or environmental sample. The sample is preferably a biological sample. A biological sample may be understood to mean a sample derived from organic matter, preferably a sample obtained from a living organism. Biological samples may include organ samples, tissue samples, cellular samples, fluid samples or any other sample obtainable, obtained, derivable or derived from an organism or a component of the organism. For example, the biological sample can comprise a fermentation medium, feedstock or food product such as for example, but not limited to, dairy products. Suitably, the enzyme activity of the biosensor is not substantially inhibited by components of the sample (e.g. serum proteins, metabolites, cells, cellular debris and components, naturally-occurring protease inhibitors etc). The biological sample may be obtained from animals, (preferably mammals, most preferably humans), plants or fungi. The sample may be obtained from agriculturally important animals or plants, such as livestock (e.g., cows, sheep, pigs, chickens, etc.) or crops (e.g., vegetables, grains, or other foodstuffs). Preferably, the sample may be a biological sample, wherein the biological sample is selected from: tissue, blood, plasma, serum, saliva, interstitial fluid, milk, juice, sap, or homogenate. The biological sample may be obtainable or obtained from a mammal, preferably a human. The biological sample may be a fluid sample such as blood, serum, plasma, urine, saliva, tears, sweat, cerebrospinal fluid or amniotic fluid. The sample may be a tissue sample such as a tissue or organ biopsy or may be a cellular sample such as a sample comprising red blood cells, lymphocytes, tumour cells or skin cells. A particular type of biological sample is a pathology sample. The sample may be a blood, saliva, serum or urine sample from a human subject. The sample may be a blood or saliva sample obtained from a human. The sample may be a serum or urine sample from a human subject. The human may a patient. The human may have or may be suspected of having a disease for which the one or more target molecules is/are a marker or biomarker. An environmental sample may be understood to mean a sample derived from inorganic matter, such as a water sample, soil sample, rock sample or air sample.

In some instances, the biosensor and/or methods of use may be applicable to drug testing such as for detecting the use of illicit drugs of addiction (e.g., cannabinoids, amphetamines, cocaine, heroin etc.) and/or for the detection of performance-enhancing substances in sport and/or masking agents that are typically used to avoid detection of performance-enhancing substances. This may be applicable to the detection of banned performance-enhancing substances in humans and/or other mammals such as racehorses and greyhounds that may be subjected to illicit “doping” to enhance performance.

The biosensors of the invention may also be used to screen for proteins that bind to specific target molecules. For example, in some instances, the sample may comprise a known purified target molecule, such as a target protein, a target peptide or a target small molecule. A plurality of biosensors may be provided comprising a panel of different pairs of binding moieties to be screened for binding to the one or more target molecules. As described herein, specific binding of the pairs of binding moieties to a target molecule would result in activation of the biosensor.

Also described herein is a method of diagnosis of a disease or condition in an organism, said method comprising the step of contacting one or more reporter proteins as described herein, or one or more biosensors as described herein, with a sample obtained from the organism under conditions suitable for detection of the presence or absence of one or more target molecules in the sample, wherein the presence or absence of the one or more target molecules in the sample is indicative of whether the organism has, or is at risk of having, said disease or condition. Typically, the one or more target molecules are either one target molecule or two target molecules. In some instances, determination of the presence or absence of the one or more target molecules facilitates diagnosis of the disease or condition. The organism may include plants and animals inclusive of fish, avians and mammals such as humans. Preferably, the organism is a mammal, most preferably a human.

The disease or condition may be any disease or condition where detection of a target molecule assists diagnosis. Suitable target molecules are described herein. Target molecules may include one or more of blood coagulation factors such as previously described, kallikreins inclusive of PSA, matrix metalloproteinases, viral and bacterial proteases, antibodies, glucose, triglycerides, lipoproteins, cholesterol, tumour antigens, lymphocyte antigens, autoantigens and autoantibodies, drugs, salts, creatinine, blood serum or plasma proteins, pesticides, uric acid, products and intermediates of human and animal metabolism and metals. Target molecules preferably include one or more of methotrexate, phenolic glucosides, Rapamycin,, tacrolimus, or Cyclosporine A. These methods may be adapted to be performed as “point of care” methods whereby determination of the presence or absence of the target molecule may occur at a patient location which is then either analysed at that location or transmitted to a remote location for diagnosis of the disease or condition.

Also described herein is a method of monitoring one or more target molecules in an organism, said method comprising the step of contacting one or more reporter proteins as described herein, or one or more biosensors as described herein, with a sample obtained from the organism under conditions suitable for detection of the presence or absence of one or more target molecules in the sample. Multiple samples may be taken at regular intervals over a defined period of time, such over a day, over a week, over a month, or over a year, in order to monitor the presence or absence of the target molecule in the organism over said period of time. The method may further comprise quantifying the level of the target molecule in the organism. The method may further comprise detecting and quantifying the target molecule in the same. The method may further comprise monitoring the level of the one or more target molecules in the organism, for example over a period of time, wherein the period of time may be a day, a week, a month, a year or multiple years. The method may allow for monitoring different states of normal physiology in the organism. Thus, the presence or absence of the one or more target molecules in the sample may be indicative of a particular physiological state in the organism. The level (i.e., the quantity or concentration) of the one or more target molecules in the sample may be indicative of a particular physiological state in the organism.

In some aspects, the method may be for monitoring the fertility cycle, and the one or more target molecules are hormones, such as progesterone, estradiol, luteinising hormone (LH) and follicle stimulating hormone (FSH). The method may be for monitoring levels of psychological stress, and the one or more target molecules are cortisol or α-amylase. The method may be for monitoring metabolic state, and the one or more target molecules are insulin and glucose. The method may be for monitoring levels of therapeutic drugs, such as methotrexate, rapamycin, tacrolimus, or cyclosporine A. The method may be for monitoring levels of environmental contaminants, such as phenolic glucosides. The organism may be a plant (such as an agriculturally important plant, such as a crop) or an animal, including e.g., fish, birds, mammals (such as an agriculturally important animal, such as livestock). The organism may be a mammal. Preferably the organism is a human.

Also described herein is a kit or device adapted to perform the methods described herein. Described herein is a kit comprising one or a plurality of different biosensors as described herein capable of detecting one or a plurality of different target molecules. In this regard, a kit may comprise an array of different biosensors as described herein capable of detecting a plurality of different target molecules. The kit may further comprise one or a plurality of suitable substrates of the reporter protein(s) of the biosensor, as described herein. The kit may also comprise additional components including reagents such as buffers and diluents, reaction vessels and instructions for use.

In some instances, the reporter proteins or biosensors as described herein may be used to assay for protein-protein or protein-small molecule binding interactions. Thus, described herein is a method of assaying protein-protein or protein-small molecule interactions comprising contacting the reporter proteins or biosensors as described herein with a sample under conditions suitable for detection of the presence or absence of an interaction between a pair of binding moieties or between a pair of binding moieties and their corresponding target molecule. The sample typically comprises a suitable substrate molecule for the reporter protein or biosensor.

In the biosensors of the invention, the activity of the reporter protein may be dependent upon a specific protein-protein or protein-small molecule interaction between the pairs of binding moieties (i.e., no target molecule is required). Such a biosensor may be used to assay for a direct interaction between a first protein or small molecule of interest (i.e., one half of the pair of binding moieties) and a second protein or small molecule of interest (i.e., the corresponding other half of the pair of binding moieties). A direct interaction between the proteins/small molecules of interest in the binding moiety on the reporter protein and the binding corresponding binding moiety on a regulatory moiety (which form a pair of binding moieties) would co-localise the reporter protein with a regulatory moiety, facilitating binding of the regulatory moiety to the heterologous amino acid sequence of the reporter protein, and thereby activating the activity of the reporter protein, as described herein, to produce a detectable read-out as described herein. Such a biosensor may be used to assay for a direct interaction between the two halves of each pair of binding moieties in the biosensor. Such a biosensor may be further used to assay for activators and inhibitors of the said interaction between the two halves of the pairs of binding moieties, which may be proteins or small molecules. Thus, a sample may further comprise putative activators and inhibitors, i.e., molecules to be assayed for their ability to activate or inhibit the interaction between a pair of binding moieties. Activators that enhance the interaction between a pair of binding moieties would result in increased activity of the reporter protein. Inhibitors that inhibit, prevent or reduce the interaction between a pair of binding moieties would result in reduced activity of the reporter protein.

Also described herein is a method for converting an enzyme into a reversibly regulated enzyme whose catalytic activity is dependent on the presence of one or more target molecules. The method may comprise in a first step (a) generating a library of single insert enzyme mutants by inserting a heterologous amino acid sequence which is responsive to binding of a regulator moiety at a number of different locations in an enzyme sequence.

A single insert enzyme mutant may be generated by introducing a heterologous amino acid sequence at a particular location within the amino acid sequence of the enzyme. Typically, the heterologous amino acid sequence is inserted at a particular location within the amino acid sequence of the enzyme, typically between two contiguous residues in the amino acid sequence of the enzyme. The skilled person would be readily capable of carry out such insertions, for example using recombinant DNA manipulation (e.g., using restriction enzymes, ligation, and PCR techniques) and recombinant protein expression techniques, which are well-known in the art. A plurality of single enzyme mutants, wherein each single enzyme mutant comprises one heterologous amino acid sequence inserted at a particular location, may be generated to form a library of single insert enzyme mutants. Typically, each single insert enzyme mutant in the library comprise the heterologous amino acid sequence inserted at a different location within the amino acid sequence of the enzyme. The enzyme and the heterologous amino acid sequence are typically provided as a contiguous amino acid sequence. For example, a single insert enzyme mutant may comprise from N- to C-terminus a first portion of the enzyme sequence, the heterologous amino acid sequence, and a second portion of the enzyme sequence. Typically the first and second portions of the enzyme sequence correspond substantially to the amino acid sequence of the wild type enzyme. The heterologous amino acid sequence which is responsive to binding of a regulator moiety may be any heterologous amino acid sequence as described herein. Preferably, the heterologous amino acid sequence is a calmodulin protein or functional fragment thereof, as described herein, and the regulator moiety is a calmodulin binding peptide (CaM-BP), as described herein.

The method may further comprise assaying the catalytic activity of the library of single insert enzyme mutants. Thus, the catalytic activity of each of the single insert enzyme mutants within the library may be assayed. Such an assay may include contacting a single insert enzyme mutant with its substrate under conditions suitable for catalytic activity of the enzyme. The signal or read-out of the catalytic activity of the enzyme may be detected, measured and/or quantified. The skilled person would be readily capable of selecting a suitable assays for detecting, measuring and/or quantifying the catalytic activity of the an enzyme. Typically, the activity assay is performed in the presence and absence of the regulator moiety. The method may further comprise selecting the single insert enzyme mutants that show a change in catalytic activity on binding of the regulator moiety, usually in the presence of a suitable enzyme substrate and under suitable reaction conditions. Preferably single insert enzyme mutants that show a change in catalytic activity on binding of the regulator moiety within a desired time window. For example, single insert enzyme mutants showing a fast response time to the addition of the regulator moiety are desirable. The selected single insert enzyme mutants may show a change in catalytic activity (i.e., a significant change, such as a catalytic activity at least two fold the catalytic activity in the absence of regulator moiety) within 30 seconds of addition of the regulator moiety. The single insert enzyme mutants preferably have a dynamic range at least 2, 3, 4, 5, 6, 7, 8, 9, 10, 12, 15, or at least 20 fold, preferably between 2 and 20 fold, most preferably between 5 and 15 fold. The desired time window may be less than 30 minutes, 20 minutes, 15 minutes, 10 minutes, 5 minutes, 2 minutes, or less than 1 minute, most preferably less than 10 minutes. The change in catalytic activity that occurs in the desired time window is typically at least 2×, 3×, 4×, 5×, 6×, 7×, 8×, 9×, or at least 10× the catalytic activity in the absence of regulator moiety (i.e., signal change), preferably the change in catalytic activity that occurs in the desired time window is at least 5×, most preferably at least 10×.

The method may further comprise a step (b) generating a library of double insert enzyme mutants by inserting an additional heterologous amino acid sequence which is responsive to binding of a regulator moiety at a second site within the enzyme sequence. A double insert enzyme mutant may be understood to comprise two heterologous amino acid sequences typically inserted within the amino acid sequence of the enzyme, typically at different locations within the enzyme amino acid sequence. The enzyme and the two heterologous amino acid sequences are typically provided as a contiguous amino acid sequence. For example, a double insert enzyme mutant may comprise from N- to C-terminus a first portion of the enzyme sequence, a first heterologous amino acid sequence, a second portion of the enzyme sequence, a second heterologous amino acid sequence, and a third portion of the enzyme sequence. Typically the first, second and third portions of the enzyme sequence correspond substantially to the amino acid sequence of the wild type enzyme. Each heterologous amino acid sequence may be a heterologous amino acid sequence as described herein. The two heterologous amino acid sequences may be the same or different. Each of the two heterologous amino acid sequences may comprise or consist of a calmodulin protein, or a functional fragment thereof. The library of double insert enzyme mutants typically comprises a plurality of double insert enzyme mutants wherein each double insert enzyme mutant within the library comprises the two heterologous amino acid sequences inserted at different locations. For each double insert enzyme mutant, the insertion location of one of the heterologous amino acid sequences may be the same and the other insertion location different. Or for each double insert enzyme mutant, the insertion locations of the two heterologous amino acid sequences may both be different. In some instances the insertion locations for the double insert mutants may be selected by combining the insertion locations found to have the fastest response times of the single insert enzyme mutants.

The method may further comprise the step of selecting those double insert enzyme mutants showing a change in catalytic activity in the presence of the regulator moiety and having an increased dynamic range within a desired time window, as compared to the corresponding single insert enzyme mutants. Thus, those double insert enzyme mutants are selected that upon addition of the regulator moiety show a change in catalytic activity, usually in the presence of enzyme substrate and under appropriate reaction conditions. As discussed above, the skilled person would be readily capable of selecting a suitable assay for catalytic activity of a particular enzyme. The change in catalytic activity upon addition of the regulator moiety of the double insert mutant enzyme may be compared to the change in catalytic activity upon addition of the regulator moiety of the corresponding single insert mutant enzymes, i.e., the single insert mutant enzymes having a heterologous amino acid sequence inserted at a location corresponding to one of the two locations at which the heterologous amino acid sequence is inserted in the double insert mutant enzyme. Preferably, those double insert mutant enzymes are selected that show an improved dynamic range within a desired time window, as compared to either and preferably both of the single insert mutant enzymes. The dynamic range of the double insert mutant enzyme may be at least 2×, 3×, 4×, 5×, 6×, 7×, 90×, 10×, 20×, 30×, 40×, 50×, 75× or 100×, preferably at least 10×, most preferably at least 25X the dynamic range of either and preferably both of the single insert mutant enzymes. The desired time window may be less than 30 minutes, 20 minutes, 15 minutes, 10 minutes, 5 minutes, 2 minutes, or less than 1 minute, most preferably less than 10 minutes. The change in catalytic activity that occurs in the desired time window is typically at least 2×, 3×, 4×, 5×, 6×, 7×, 8×, 9×, or at least 10× the catalytic activity in the absence of regulator moiety (i.e., signal change), preferably the change in catalytic activity that occurs in the desired time window is at least 5×, most preferably at least 10×. Thus, preferably, for example for clinical applications, the reporter proteins or biosensors described herein may show at least 5× signal change (i.e., change in catalytic activity upon addition of the regulator moiety, as compared to the catalytic activity in the absence of regulator moiety) in 20 minutes or less, most preferably the reporter proteins or biosensors described herein may show at least 10× signal change in 10 minutes or less.

Thus, described herein is a method for converting a constitutively active enzyme into a reversibly regulated enzyme whose catalytic activity is dependent on the presence of one or more target molecules, the method comprising:

(a) generating a library of single insert enzyme mutants by inserting a heterologous amino acid sequence which is responsive to binding of a regulator moiety at a number of different locations in an enzyme sequence and selecting those single insert enzyme mutants showing a change in catalytic activity in a desired time window on binding of the regulator moiety;
(b) generating a library of double insert enzyme mutants by inserting an additional heterologous amino acid sequence which is responsive to binding of a regulator moiety at a second site within the enzyme sequence, and selecting those double insert enzyme mutants showing a change in catalytic activity in the presence of the regulator moiety and having an increased dynamic range as compared to the corresponding single insert enzyme mutants, preferably wherein the double mutant insert enzyme also shows a change in catalytic activity in the desired time window.

### Nucleic Acids

The present invention also provides one or more isolated nucleic acid(s), encoding a reporter protein of the invention, a biosensor of the invention, or a component of a biosensor of the invention. The nucleic acid may encode any of SEQ ID NOs: 1 to 67, 69, 70, or 73 to 82, or a variant thereof. The nucleic acid may encode an amino acid sequence comprising the sequences of one or more of, or any of, SEQ ID NOs: 1 to 67, 69, 70, or 73 to 82. The nucleic acid may comprise SEQ ID NO: 68 or 71, or variants thereof.

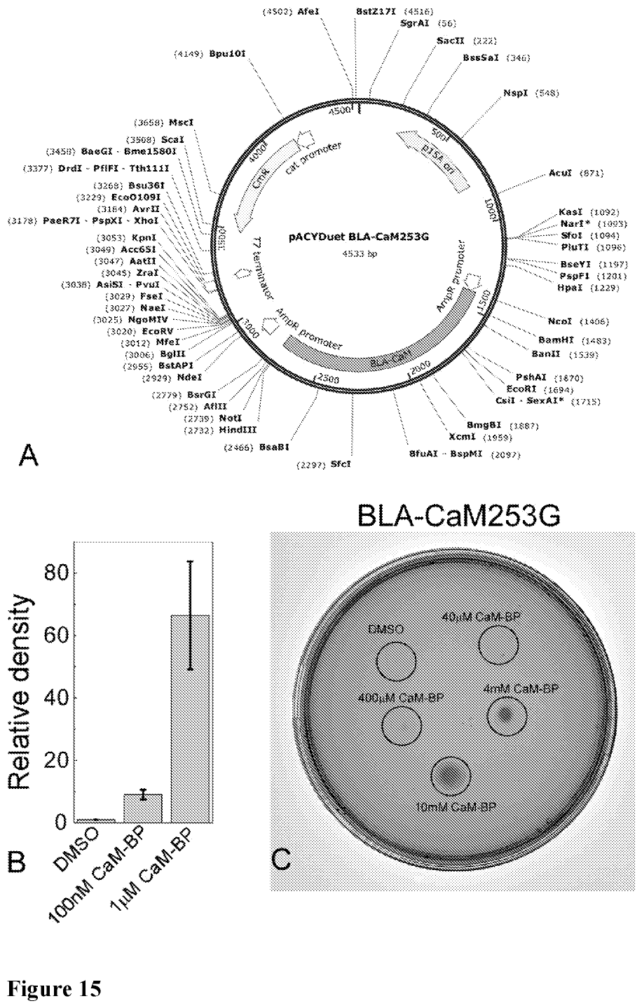

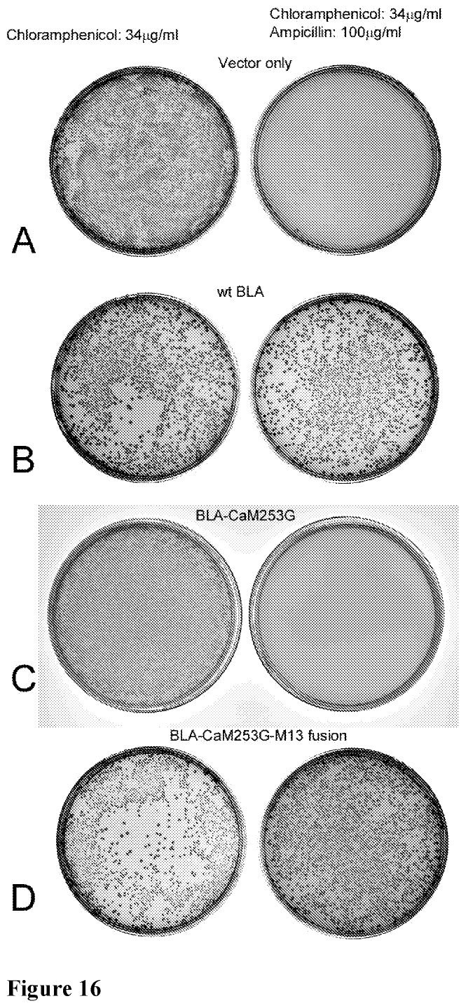

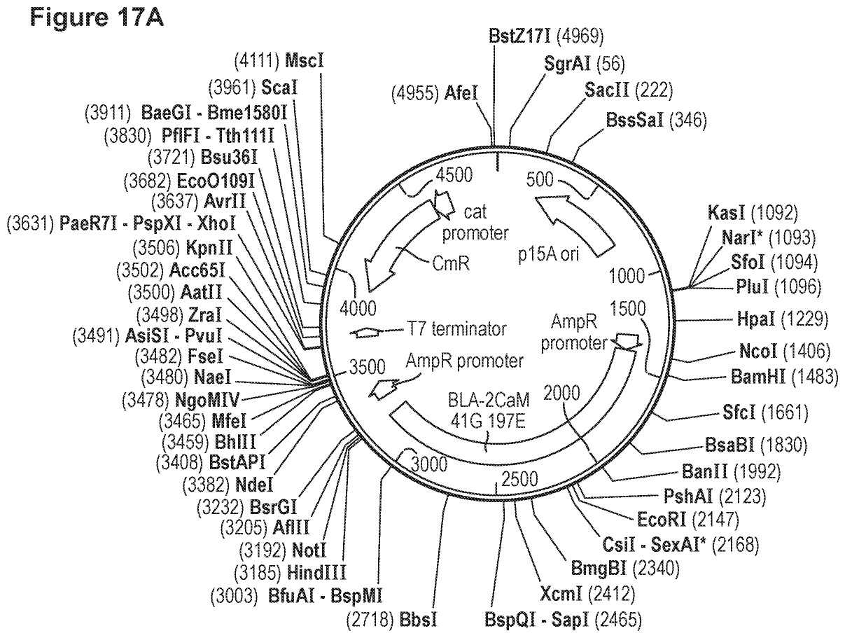

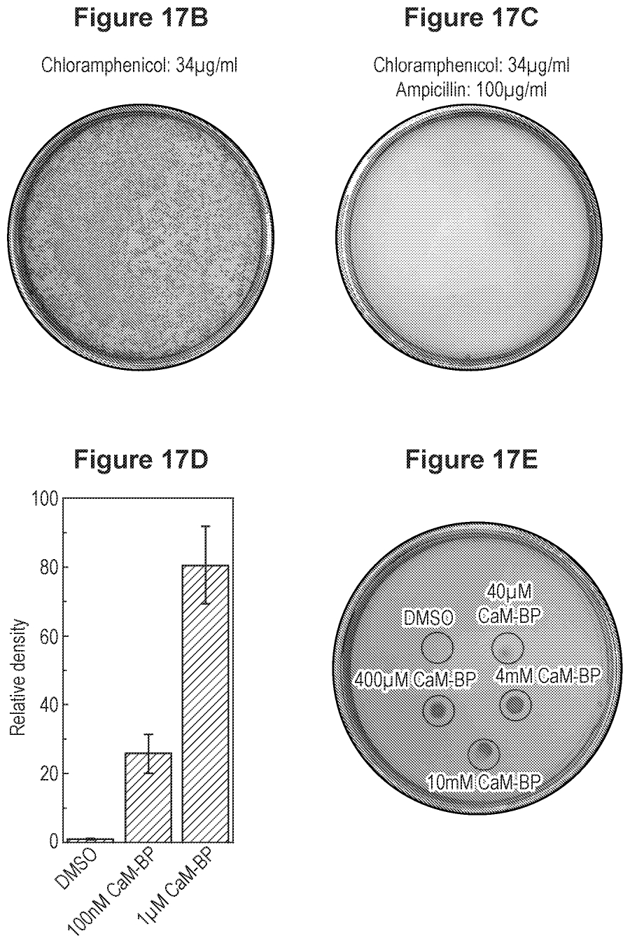

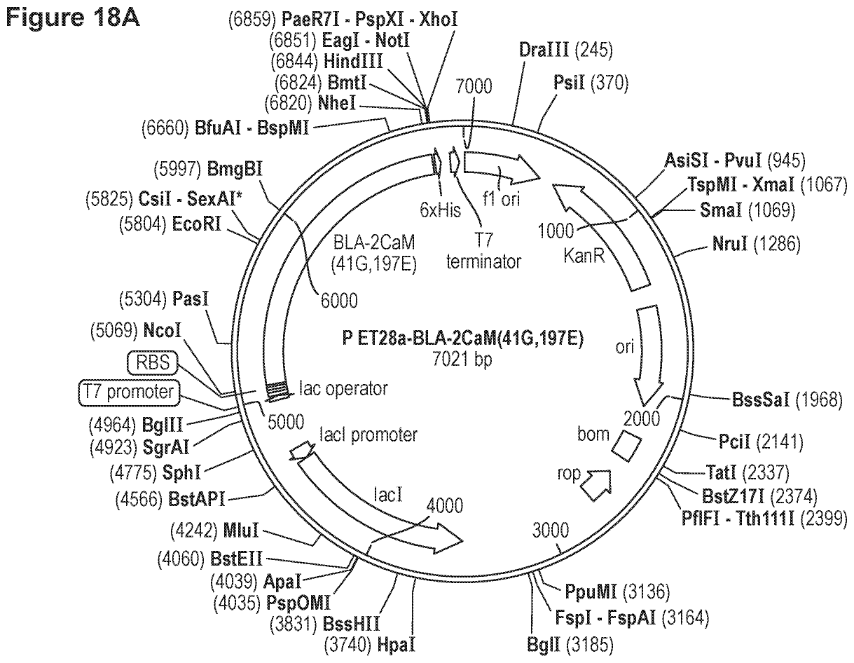

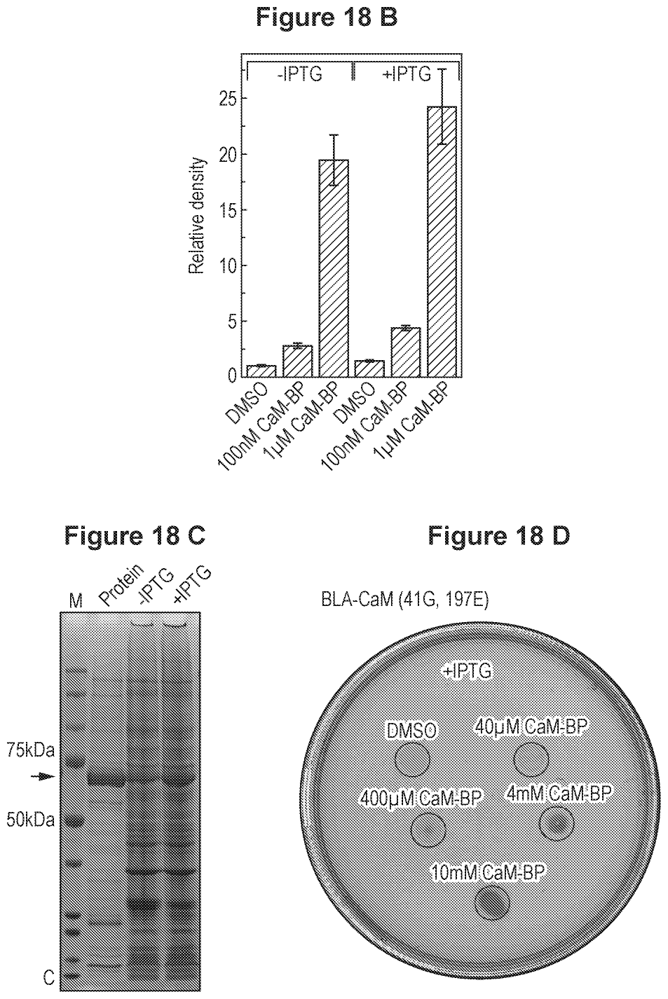

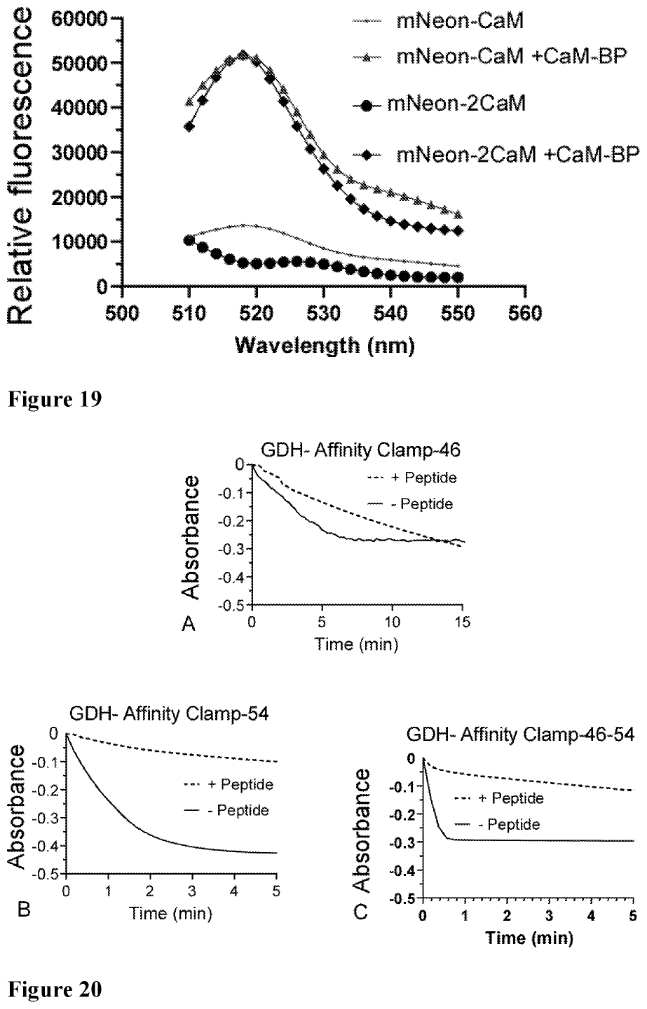

Described herein is one or more genetic construct(s) comprising the one or more isolated nucleic acid(s) of the invention. Described herein is one or more vector(s) comprising the one or more isolated nucleic acid(s) of the invention. Also described herein is one or more vector(s) suitable for, or adapted for, expression of a reporter protein as described herein, a biosensor as described herein or a component of a biosensor as described herein, for example in a suitable host cell (see, e.g., SEQ ID NO: 68 or 71). Also described herein is a host cell comprising the one or more isolated nucleic acid(s), the one or more genetic construct(s), or the one or more vector(s) described herein (see, e.g., FIGS. 15-18).

The term “nucleic acid” as used herein designates single- or double-stranded mRNA, RNA, CRNA, RNAi, siRNA and DNA inclusive of cDNA, mitochondrial DNA (mtDNA) and genomic DNA. Preferably, the nucleic acids of the invention are DNA. The invention also provides variants and/or fragments of the isolated nucleic acids. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence disclosed herein. In other instances, nucleic acid variants may hybridize with any nucleotide sequence described herein, under high stringency conditions. Fragments may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence described herein. Fragments may comprise or consist of up to 20, 50, 100, 150, 200, 250, 300, 350, 400, 450, 500, 550, 600, 650, 700, 750, 800, 850, 900 950, 1000, 1050, 1100, 1150, 1200, 1350 or 1300 contiguous nucleotides present in any nucleotide sequence described herein.

The isolated nucleic acid(s) may be operably linked to one or more additional nucleotide sequences, such as one or more promoter(s) and/or one or more enhancers. The one or more additional nucleotide sequences are typically regulatory nucleotide sequences. By operably linked or operably connected is meant that said regulatory nucleotide sequence(s) is/are positioned relative to the nucleic acid to be expressed to initiate, regulate or otherwise control expression of the nucleic acid. As generally used herein, a “genetic construct” is an artificially created nucleic acid that incorporates, and/or facilitates use of, an isolated nucleic acid disclosed herein. In particular instances, such constructs may be useful for recombinant manipulation, propagation, amplification, homologous recombination and/or expression of said isolated nucleic acid.

Regulatory nucleotide sequences will generally be appropriate for the host cell used for expression. Numerous types of appropriate expression vectors and suitable regulatory sequences are known in the art for a variety of host cells. One or more regulatory nucleotide sequences may include, but are not limited to, promoter sequences, leader or signal sequences, ribosomal binding sites, transcriptional start and termination sequences, translational start and termination sequences, splice donor/acceptor sequences and enhancer or activator sequences. Constitutive or inducible promoters as known in the art may be used and include, for example, nisin-inducible, tetracycline-repressible, IPTG-inducible, alcohol-inducible, acid-inducible and/or metal-inducible promoters. In one instances, the expression vector comprises a selectable marker gene. Selectable markers may be useful for the purposes of selection of transformed bacteria (such as bla, kanR, ermB and tetR) or transformed mammalian cells (such as hygromycin, G418 and puromycin resistance).

Also described herein is a method of producing (i) a reporter protein of the invention, or (ii) a biosensor, or component thereof, of the invention, said method including a step of expressing said reporter protein, biosensor, or a component thereof, in a suitable host cell. host cell of the invention. Also described herein is a method of producing (i) a reporter protein of the invention, or (ii) a biosensor, or component thereof, of the invention, said method including a step of introducing one or more nucleic acid(s) of the invention into a suitable host cell and incubating the host cell under conditions suitable for expression of said reporter protein, biosensor, or a component thereof. The method may be a recombinant expression method. As used herein, a genetic construct used for recombinant protein expression is referred to as an expression construct, wherein the isolated nucleic acid to be expressed is operably linked or operably connected to one or more additional nucleotide sequences. in an expression vector. An expression vector may be either a self-replicating extra-chromosomal vector such as a plasmid, or a vector that integrates into a host genome.

Suitable host cells for expression may be prokaryotic or eukaryotic, such as bacterial cells inclusive of Escherichia coli (DH5α for example), yeast cells such as S. cerivisiae or Pichia pastoris, insect cells such as SF9 cells utilized with a baculovirus expression system, or any of various mammalian or other animal host cells such as CHO, BHK or 293 cells. Introduction of expression constructs into suitable host cells may be by way of techniques including, for example, electroporation, heat shock, calcium phosphate precipitation, DEAE dextran-mediated transfection, liposome-based transfection (e.g. lipofectin, lipofectamine), protoplast fusion, microinjection or microparticle bombardment, as are well known in the art.

Purification of the recombinantly-produced reporter protein, biosensor or component of a biosensor, may be performed by any method known in the art. In preferred instances, the recombinant protein molecule comprises a fusion partner (preferably a C-terminal His tag) which allows purification by virtue of an appropriate affinity matrix, which in the case of a His tag would be a nickel matrix or resin. The resulting, engineered mutant is preferably expressed in bacteria such as E.coli as an epitope-tagged protein and is purified by affinity chromatography.

The reporter proteins or biosensors of the invention may be expressed in host cells, for example for metabolic engineering applications. This is illustrated for example in FIGS. 15-17. Suitable host cells in which the reporter proteins and/or biosensors of the invention may be expressed, for metabolic engineering applications may be prokaryotic or eukaryotic. The host cells may be selected from microbial cells, bacterial cells (inclusive of Escherichia coli, such as DH5α for example), yeast cells (such as S. cerivisiae or Pichia pastoris), insect cells (such as SF9 cells utilized with a baculovirus expression system), plant cells (such as cells of wheat, maize or other crops), or mammalian cells (such as CHO, BHK or 293 cells). Preferably the host cell is a microbial cell, a bacterial cells (such as, preferably, an E. coli cell) or a yeast cell, most preferably a bacterial cell. Introduction of constructs for expressing the reporter proteins/biosensors described herein may be introduced into suitable host cells by any suitable technique known in the art and depending on the host cell, such as for example, electroporation, heat shock, calcium phosphate precipitation, DEAE dextran-mediated transfection, liposome-based transfection (e.g. lipofectin, lipofectamine), protoplast fusion, microinjection or microparticle bombardment.

For example, the reporter proteins/biosensors may be used to trigger or control a certain property of the host cell in response to one or more target molecules. For example, certain reporter proteins/biosensors that are based on antibiotic resistance markers (e.g., wherein the reporter protein is β-lactamase or aminoglycoside phosphotransferase) may be used to construct biosensors that induce antibiotic resistance in the host cells, which is typically a microbial cells (e.g., a bacterial cell, a yeast cell, an E. coli cell, etc.), in response to the one or more target molecules of the biosensor. For example, to select cells that produce high levels of a particular compound or protein of interest, a biosensor based on an antibiotic resistance marker, having the compound or protein of interest as a target molecule is prepared. The cells being tested are mutagenized to express the biosensors, challenged with the relevant antibiotic, and the surviving cell colonies are selected, which will comprise cells producing enough of the target molecule to activate the biosensor and turn on enough antibiotic resistance to survive. For example, see FIGS. 15-17. In such applications, the large dynamic ranges of the biosensors of the invention are very important, so the host cell bacteria are not able to survive simply by producing more of the low activity resistance protein. Similarly, the reporter proteins/biosensors may be based on fluorescent (e.g., FRET (fluorescence resonance energy transfer) systems) or luminescent proteins (e.g., BRET (bioluminescence resonance energy transfer) systems), and may be used to construct biosensors that induce fluorescence or luminescence in the host cells in response to the one or more target molecules of the biosensor. For example, to select cells that produce high levels of a particular compound or protein of interest, a biosensor based on a fluorescent or luminescent protein, having the compound or protein of interest as a target molecule is prepared. The cells being tested are mutagenized to express the biosensor(s), and positive close may be detected by high levels of fluorescence/luminescence and may be isolated for example using fluorescence-activated cell sorting (FACS). The selected cells are those producing enough of the target molecule to activate the biosensor and turn on high levels of fluorescence/luminescence.

Another application of the biosensors of the invention is metabolic switching. For example, in certain application it is not desirable to produce a certain product until the host cells have grown to an optimum or maximal density. Biosensors according to the invention are prepared that have as a target molecule a cell metabolite whose production or high concentration is indicative of optimum or maximal cell density. The biosensor is expressed in the host cells and when the target metabolite reaches the desired level the biosensor is activated with the read-out or signal from the biosensor indicating that the cells are ready to be used for production of the desired product.

Further metabolic engineering applications would be evident to the skilled person who, following the teachings of the present invention, would be readily capable of preparing suitable reporter proteins and biosensors tailored to the specific application.

The invention may be better understood by reference to the following Examples.

### ASPECTS OF THE INVENTION

1. A reporter protein comprising a first heterologous amino acid sequence which is responsive to binding of a first regulator moiety and a second heterologous amino acid sequence which is responsive to binding of a second regulator moiety, wherein binding of the first regulator moiety to the first heterologous amino acid sequence and binding of the second regulator moiety to the second heterologous amino acid sequence, reversibly regulates the activity of the reporter protein.

2. The reporter protein of aspect 1, wherein the reporter protein is an enzyme and wherein the activity of the reporter protein is the catalytic activity of the enzyme; or wherein the reporter protein is a fluorescent protein and wherein the activity of the reporter protein is the fluorescence of the fluorescent protein.

3. The reporter protein of aspect 1 or 2, wherein binding of the first regulator moiety to the first heterologous amino acid sequence and binding of the second regulator moiety to the second heterologous amino acid sequence, reversibly activates the activity of the reporter protein.

4. The reporter protein of any one of aspects 1-3, wherein the dynamic range of the reporter protein is at least 10 fold, optionally at least 20 fold.

5. The reporter protein of any one of aspects 1-4, wherein the first heterologous amino acid sequence is provided as an insert within the amino acid sequence of the reporter protein and the second heterologous amino acid sequence is provided as an insert within the amino acid sequence of the reporter protein.

6. The reporter protein of any one of aspects 1-5, wherein the first heterologous amino acid sequence and the second heterologous amino acid sequence are inserted at different locations within the amino acid sequence of the reporter protein.

7. The reporter protein of any one of aspects 1-6, wherein the first heterologous amino acid sequence and/or the second heterologous amino acid sequence is a protein.

8. The reporter protein of any one of aspects 1-7, wherein the first heterologous amino acid sequence and the second heterologous amino acid sequence are proteins that are capable of undergoing a conformational change in response to binding of a regulator moiety.

9. The reporter protein of any one of aspects 1-8, wherein the first heterologous amino acid sequence and the second heterologous amino acid sequence are the same protein, or a functional fragment thereof.

10. The reporter protein of any one of aspects 1-9, wherein the first heterologous amino acid sequence and/or the second heterologous amino acid sequence is a calmodulin protein or an affinity clamp, or a functional fragment thereof.

11. The reporter protein of any one of aspects 1-10, wherein the reporter protein further comprises one or more further heterologous amino acid sequences.

12. The reporter protein of any one of aspects 1-11, wherein one or more further heterologous amino acid sequences are provided as inserts within the amino acid sequence of the reporter protein.

13. The reporter protein of any one of aspects 1-12, wherein each heterologous amino acid sequence is inserted within the amino acid sequence of the reporter protein at a different insertion location.

14. The reporter protein of any one of aspects 1-13, wherein binding of a regulator moiety to the first heterologous amino acid sequence causes a conformational change in the structure of the first heterologous amino acid sequence and wherein binding of a regulator moiety to the second heterologous amino acid sequence causes a conformational change in the structure of the second heterologous amino acid sequence.

15. The reporter protein of any one of aspects 1-14, wherein the first regulator moiety and/or second regulator moiety is a peptide.

16. The reporter protein of any one of aspects 1-15, wherein the first regulator moiety and the second regulator moiety are the same peptide.

17. The reporter protein of any one of aspects 1-16, wherein the first regulator moiety and/or second regulator moiety is a calmodulin-binding peptide or an affinity clamp RGS peptide ligand.

18. The reporter protein of any one of aspects 1-17, wherein the first regulator moiety and/or second regulator moiety is a calmodulin-binding peptide having a reduced binding affinity for a calmodulin protein, as compared to a wild-type calmodulin-binding peptide.

19. The reporter protein of aspect 18, wherein the first and second regulator moieties are linked together, optionally through a linker, optionally as a fusion protein.

20. The reporter protein of aspect 19, wherein the first and second regulator moieties are each further linked to a protein which is a variant of the first or second heterologous amino acid sequence, respectively, having a reduced binding affinity for the regulator moiety, as compared to the binding affinity of the respective heterologous amino acid sequence on the reporter protein.

21. The reporter protein of aspect 20, wherein (i) the first and second heterologous amino acid sequences are calmodulin proteins, and (ii) the first and second regulator moieties are calmodulin-binding peptides, each further linked to a calmodulin protein having a reduced binding affinity for the calmodulin-binding peptide, as compared to the binding affinity of the calmodulin protein on the reporter protein.

22. The reporter protein of any one of aspects 1-21, wherein binding of a calmodulin-binding peptide to the first and second heterologous amino acid sequences which are both calmodulin, activates the activity of the reporter protein.

23. The reporter protein of any one of aspects 1-22, (i) wherein the reporter protein is an enzyme selected from the group consisting of trehalase, an oxidoreductase, glucose dehydrogenase, β-lactamase, aminoglycoside phosphotransferase, α-amylase, and carbonic anhydrase; or (ii) wherein the reporter protein is a fluorescent protein selected from the group consisting of GFP, Cherry, mNeon, a bacterial phytochrome (BphP)-based fluorescent protein, or a cyanobacteriochrome (CBCR)-derived fluorescent protein.

24. The reporter protein of any one of aspects 1-23, wherein the reporter protein further comprises a first binding moiety B1′.

25. The reporter protein of any one of aspects 1-24, wherein the first and second regulator moieties are linked together and further linked to a binding moiety B1″, which is capable of interacting with a first binding moiety B1′ on the reporter protein.

26. The reporter protein of aspect 25, wherein the first and second regulator moieties and the binding moiety B1″ are linked as a fusion protein, optionally through one or more linkers.

27. The reporter protein of aspect 25, wherein interaction between binding moieties B1′ and B1″ co-localises the first regulator moiety, the second regulator moiety, and the reporter protein, thereby enhancing binding of the first and second regulator moieties to the first and second heterologous amino acid sequences, respectively, and thereby activating the activity of the reporter protein.

28. The reporter protein of any one of aspects 1-24, wherein the reporter protein further comprises a second binding moiety B2′.

29. The reporter protein of any one of aspects 1-28, wherein the first regulator moiety comprises a binding moiety B1″ which is capable of interacting with a first binding moiety B1′ on the reporter protein.

30. The reporter protein of aspect 28 or 29, wherein the second regulator moiety comprises a binding moiety B2″ which is capable of interacting with a second binding moiety B2′ on the reporter protein.

31. The reporter protein of any one of aspects 28-30, wherein interaction of the binding moieties B1′ and B1″ and interaction of the binding moieties B2′ and B2″, regulates the activity of the reporter protein, optionally activates the activity of the reporter protein.

32. The reporter protein of any one of aspects 28-31, wherein activity of the reporter protein is activated only upon interaction of both the binding moieties B1′ and B1″ and interaction of the binding moieties B2′ and B2″.

33. The reporter protein of any one of aspects 24-32, wherein interaction between binding moieties B1′ and B1″ co-localises the first regulator moiety and the reporter protein, thereby enhancing binding of the first regulator moiety to the first heterologous amino acid sequence.

34. The reporter protein of any one of aspects 28-33, wherein interaction between binding moieties B2′ and B2″ co-localises the second regulator moiety and the reporter protein, thereby enhancing binding of the second regulator moiety to the second heterologous amino acid

35. The reporter protein of any one of aspects 28-34, wherein interaction between binding moieties B1′ and B1″ and between binding moieties B2′ and B2″ co-localises the first regulator moiety, the second regulator moiety, and the reporter protein, thereby enhancing binding of the first and second regulator moieties to the first and second heterologous amino acid sequences, respectively, and thereby activating the activity of the reporter protein. sequence.

36. The reporter protein of any one of aspects 24-35, wherein binding moieties B1′ and B1″ are capable of directly binding to each other and/or binding moieties B2′ and B2″ are capable of directly binding to each other.

37. The reporter protein of any one of aspects 24-36, wherein interaction of the binding moieties B1′ and B1″ is dependent on the presence of a first target molecule TM1.

38. The reporter protein of any one of aspects 28-37, wherein interaction of the binding moieties B2′ and B2″ is dependent on the presence of a second target molecule TM2.

39. The reporter protein of any one of aspects 28-38, wherein interaction of the binding moieties B1′ and B1″ and interaction of the binding moieties B2′ and B2″ is dependent on the presence of both a first target molecule TM1 and a second target molecule TM2, such that activity of the reporter protein is activated only in the presence of both of the target molecules TM1 and TM2.

40. The reporter protein of any one of aspects 24-39, wherein in the presence of the first target molecule TM1, the first binding moiety B1′ on the reporter protein interacts with the binding moiety B1″ on the first regulator moiety.

41. The reporter protein of any one of aspects 28-40, wherein in the presence of the second target molecule TM2, the second binding moiety B2′ on the reporter protein interacts with the binding moiety B2″ on the second regulator moiety.

42. The reporter protein of any one of aspects 24-41, wherein binding moieties B1′ and B1″ form a pair of binding moieties that bind simultaneously to target molecule TM1.

43. The reporter protein of any one of aspects 28-42, wherein binding moieties B2′ and B2″ form a pair of binding moieties that bind simultaneously to target molecule TM2 .

44. The reporter protein of any one of aspects 28-43, wherein binding moieties B1′ and B1″ and binding moieties B2′ and B2″ are the same pair of binding moieties or are different pairs of binding moieties.

45. The reporter protein of any one of aspects 37-44, wherein the target molecules TM1 and TM2 are the same or different.

46. The reporter protein of any one of aspects 37-45, wherein in the presence of the first target molecule TM1 and the second target molecule TM2, the first binding moiety B1′ on the reporter protein interacts with the binding moiety B1″ on the first regulator moiety and the second binding moiety B2′ on the reporter protein interacts with the binding moiety B2″ on the second regulator moiety, wherein such co-localisation of the reporter protein and the first and second regulator moieties results in binding of the first regulator moiety to the first heterologous amino acid sequence on the reporter protein and binding of the second regulator moiety to the second heterologous amino acid sequence on the reporter protein, thereby reversibly regulating the activity of the reporter protein, optionally activating the activity of the reporter protein.

47. The reporter protein of any one of aspects 1-46, wherein the presence of a first target molecule TM1 and a second target molecule TM2 reversibly regulates activity of the reporter protein, optionally activates the activity of the reporter protein.

48. A biosensor comprising:
(a) the reporter protein of aspects 1-47
(b) a first regulator moiety as defined in any of aspects 1-47; and
(c) a second regulator moiety as defined in any of aspects 1-47.

49. A composition or kit comprising:
(a) the reporter protein of aspects 1-47; or
(b) the biosensor of aspect 48.

50. The biosensor of aspect 48 or the composition or kit of aspect 49, wherein the reporter protein is an enzyme, further comprising a substrate for the enzyme.

51. The biosensor, the composition or the kit of any one of aspects 48-50, further comprising target molecule TM1.

52. The biosensor, the composition or the kit of any one of aspects 48-51, further comprising target molecule TM2.

53. The biosensor, the composition or the kit of any one of aspects 48-52, wherein target molecule TM1 is the same as target molecule TM2; or wherein target molecule TM1 is different from target molecule TM2.

54. The biosensor, the composition or the kit of any one of aspects 48-53, further comprising a biological sample, wherein the biological sample may or may not comprise target molecule TM1 and/or target molecule TM2.

55. The biosensor, the composition or the kit of any one of aspects 48-54, wherein the reporter protein is an enzyme, further comprising a second enzyme comprising a heterologous amino acid sequence which is responsive to a peptide P, wherein binding of the peptide P to the heterologous amino acid sequence reversibly regulates catalytic activity of the second enzyme, wherein the substrate of the second enzyme is the catalytic product of the enzyme of aspects 1-47.

56. The biosensor, the composition or the kit of aspect 55, wherein (a) the second enzyme is an oxidoreductase, optionally glucose dehydrogenase, (b) the heterologous amino acid sequence is calmodulin or a functional fragment thereof, and (c) peptide P is a calmodulin-binding peptide.

57. A method of detecting one or more target molecules, comprising contacting the reporter protein of any one of aspects 1-47; the biosensor or the composition or the kit of any one of aspects 48-56; with a sample under conditions suitable for detection of the presence or absence of the one or more target molecules in the sample.

58. The method of aspect 57, wherein the one or more target molecules comprises or consist essentially of target molecule TM1 and target molecule TM2.

59. The method of aspect 57 or 58, wherein target molecule TM1 and target molecule TM2 are the same or different.

60. The method of any one of aspects 57-59, wherein the one or more target molecules is selected from the group consisting of: methotrexate, phenolic glucosides, Rapamycin, tacrolimus, and Cyclosporine A.

61. A method of diagnosis of a disease or condition in an organism, comprising contacting the reporter protein of any one of aspects 1-47; the biosensor or the composition or the kit of any one of aspects 48-56, with a sample obtained from the organism under conditions suitable for detection of the presence or absence of the one or more target molecules in the sample, wherein presence or absence of the one or more target molecules in the sample is indicative of whether the organism has, or is at risk of having, said disease or condition.

62. A method of monitoring one or more target molecules in an organism, the method comprising contacting the reporter protein of any one of aspects 1-47; the biosensor or the composition or the kit of any one of aspects 48-56, with a sample obtained from the organism under conditions suitable for detecting and/or quantifying the presence or absence of the one or more target molecules in the sample.

63. The method of aspect 62, wherein the presence or absence of the one or more target molecules in the sample is indicative of a particular physiological state in the organism; optionally wherein:
(i) the method is for monitoring the fertility cycle, and the one or more target molecules are hormones, such as progesterone, estradiol, luteinising hormone (LH) and follicle stimulating hormone (FSH);
(ii) the method is for monitoring levels of psychological stress, and the one or more target molecules are cortisol or α-amylase;
(iii) the method is for monitoring metabolic state, and the one or more target molecules are insulin and glucose;
(iv) the method is for monitoring levels of therapeutic drugs, such as methotrexate, rapamycin, tacrolimus, or cyclosporine A;
(v) the method is for monitoring levels of environmental contaminants, such as phenolic glucosides; or
(vi) the method is for monitoring a metabolite of interest.

64. A method of monitoring a metabolite of interest in an organism, the method comprising expressing the reporter protein of any one of aspects 1-47 or the biosensor of any one of aspects 48-56, in said organism under conditions suitable for detecting and/or quantifying the metabolite of interest in the organism, optionally wherein the organism is a bacterial cell.

65. A method of assaying for protein-protein or protein-small molecule interactions comprising contacting the reporter protein of any one of aspects 1-47; the biosensor or the composition or the kit of any one of aspects 48-56; with a sample under conditions suitable for detection of the presence or absence of an interaction between binding moieties B1′ and B1″ and/or an interaction between binding moieties B2′ and B2″; or an interaction between binding moieties B1′ and B1″ and a target molecule TM1 and/or an interaction between binding moieties B2′ and B2″ and a target molecule TM2.

66. The method of aspect 65, wherein the binding moieties B1′ and B1″ and the binding moieties B2′ and B2″ are proteins.

67. The method of aspect 65 or 66, wherein the target molecule TM1 and the target molecule TM2 are proteins or small molecules.

68. The method of any one of aspects 65-67, wherein binding moieties B1′ and B1″ and binding moieties B2′ and B2″ are the same pair of binding moieties, and target molecule TM1 and target molecule TM2 are the same.

69. A detection device that comprises a cell or chamber that comprises the reporter protein of any one of aspects 1-47 or the biosensor of any one of aspects 48-56.

70. One or more nucleic acids encoding the reporter protein of any one of aspects 1-47 or the biosensor of any one of aspects 48-56.

71. One or more expression vectors comprising the one or more nucleic acids of aspect 70 operably linked to one or more promoters.

72. A host cell comprising the reporter protein of any one of aspects 1-47, the biosensor of any one of aspects 48-56, the one or more nucleic acids of aspect 70, or the one or more expression vectors of aspect 71.

73. A method for converting a constitutively active enzyme into a reversibly regulated enzyme whose catalytic activity is dependent on the presence of one or more target molecules, the method comprising:
(a) generating a library of single insert enzyme mutants by inserting a heterologous amino acid sequence which is responsive to binding of a regulator moiety at a number of different locations in an enzyme sequence and selecting those single insert enzyme mutants showing a change in catalytic activity on binding of the regulator moiety;
(b) generating a library of double insert enzyme mutants by inserting an additional heterologous amino acid sequence which is responsive to binding of a regulator moiety at a second site within the enzyme sequence, and selecting those double insert enzyme mutants showing a change in catalytic activity in the presence of the regulator moiety and having an increased dynamic range as compared to the corresponding single insert enzyme mutants.

74. The method of aspect 66, wherein the change in catalytic activity occurs within less than 10 minutes, optionally less than 5 minutes.

75. The reporter protein of any one of aspects 1-17, wherein the first regulator moiety and second regulator moiety are the same calmodulin-binding peptide (CaM-BP), and wherein the first and second heterologous amino acid sequences of the reporter protein are the same calmodulin protein; optionally wherein the CaM-BP is further linked to a calmodulin protein having a reduced binding affinity for the CaM-BP, as compared to the calmodulin proteins of the reporter protein, optionally through a linker.

76. The reporter protein of aspect 18, wherein the linker linking the CaM-BP with the calmodulin protein having a reduced binding affinity comprises a protease cleavage site.

77. A biosensor comprising a reporter protein of aspect 75 or 76, and a first regulator moiety and second regulator moiety as defined in aspect 75 or 76, which is configured for detection of the protease as the target molecule.

78. A method of detecting an activity of a reporter protein of any one of aspects 1-47, wherein the reporter protein is an enzyme, using an electrochemical method, wherein the electrochemical method is capable of distinguishing the enzyme substrate from the product; optionally wherein hydrolysis of the substrate is monitored electrochemically; optionally wherein the method is performed in a biological fluid such as serum; optionally wherein the reporter protein is β-lactamase and the substrate is nitrocefin.

### EXAMPLES

As alluded to in the introduction, the ensemble nature of allosteric systems poses a principle problem to creation of states where one conformation dominates the ensemble. The design of the following exemplary biosensors of the invention was based on the assumption that in chimeric switch systems regulatory domain permits a particular array of conformations in each of its conformation states. While the active “ON” state is likely to be similar to that of the parental molecule it is expected that “OFF” states can be achieved through a broad range of mechanisms. Hence much of the engineering challenge is focused on creating an efficient and reversible “OFF” state in the reporter protein. As shown herein for the first time, conformation-induced deactivating changes in the reporter protein, created by two or more insertions of heterologous amino acid sequences at different sites, epistatically interact leading to the compounding effects demonstrated herein.

### Example 1—Trehalase-Based AND-Gate Biosensors

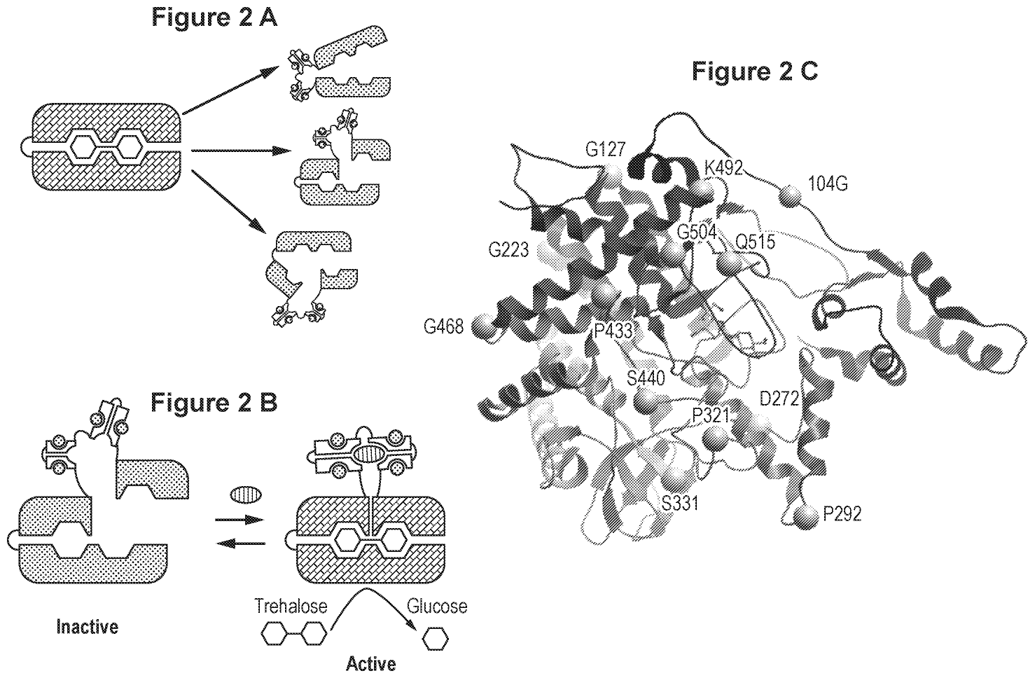

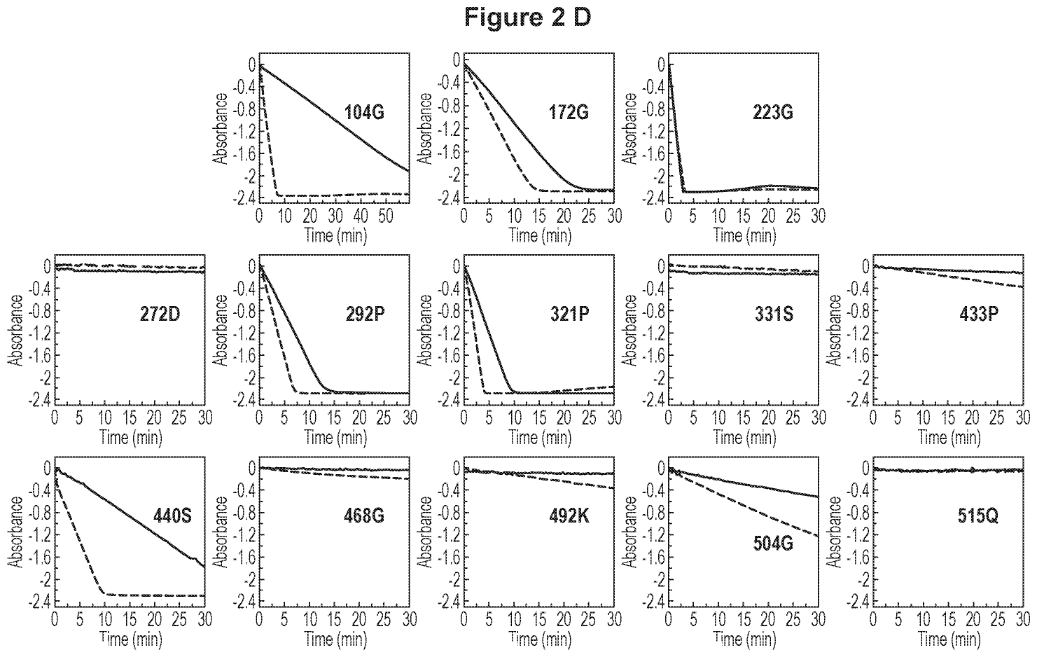

A range of insertion mutants of the enzyme trehalase and calmodulin were constructed. Trehalase degrades trehalose to glucose and its activity can be easily be monitored though a coupled glucose oxidation reaction. The trehalase structure (PDB: 2jg0) was analysed and eight Trehalase-Calmodulin (Tre-CaM) chimeras were designed. The regulatory calmodulin domain was placed at positions in the enzyme sequence where it was hypothesises that such insertions would be less likely to perturb the overall fold or the active site of the enzyme. The resulting chimeric proteins were expressed in E.coli and purified to homogeneity. The activity of the chimeric proteins was assayed using a coupled assay where trehalase generated glucose was oxidised to gluconolactone by PQQ-glucose dehydrogenase (GDH). The assay was optimised in such a way that activity of trehalase was rate limiting and therefore appeared as a first order reaction. Using this assay, the activity of the wild type trehalase as well as the Tre-CaM chimeras was monitored in the presence or absence of calmodulin binding peptide (CaM-BP). As can be seen in FIG. 2D some mutants showed little or no activity, some were active but displayed no CaM-BP dependence, and some were to different extents activated by the peptide. The switchable variants displayed modest dynamic ranges between 2 and 11 fold that were largely due to incomplete inactivation of the reporter in the absence of the ligand peptide. Analysis of selected variants confirmed that, as expected, they bound the CaM-BP with nanomolar affinity.

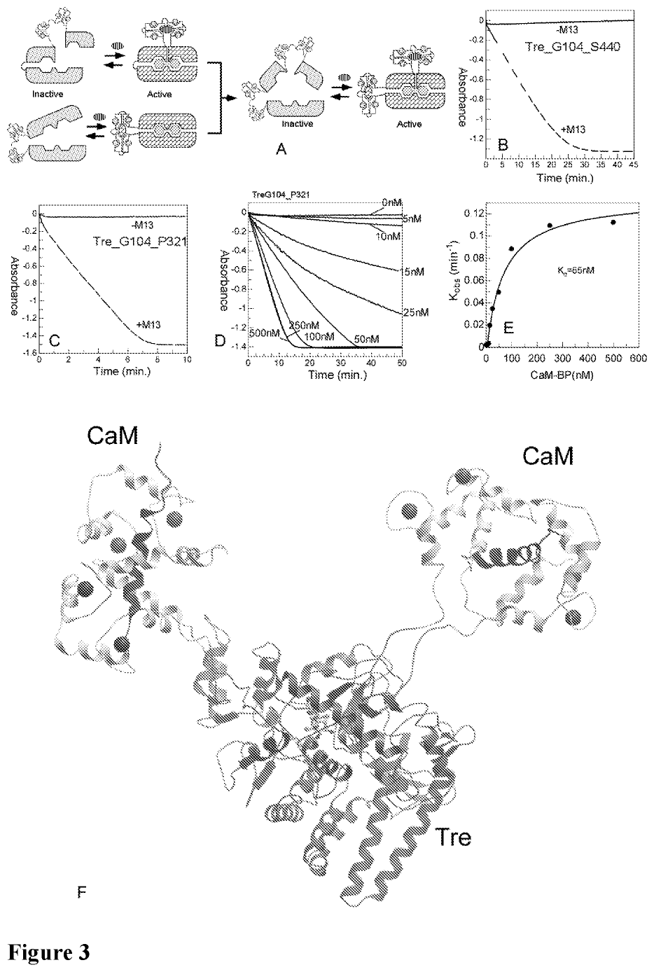

Chimeric enzymes were constructed that combined multiple insertions identified in the primary screen, in order to test for additive effects. To this end, the insertion at position 104 was combined with insertions at either position 321 or position 440 (FIG. 3A). These chimeras displayed dynamic ranges over 50 fold and bound CaM-BP saturably and dose dependently (FIGS. 3B, 3E) leading to an apparent Kd of 68 nM. This is likely to be an overestimation as the fit procedure does not take into account the dual binding site (FIG. 3F).

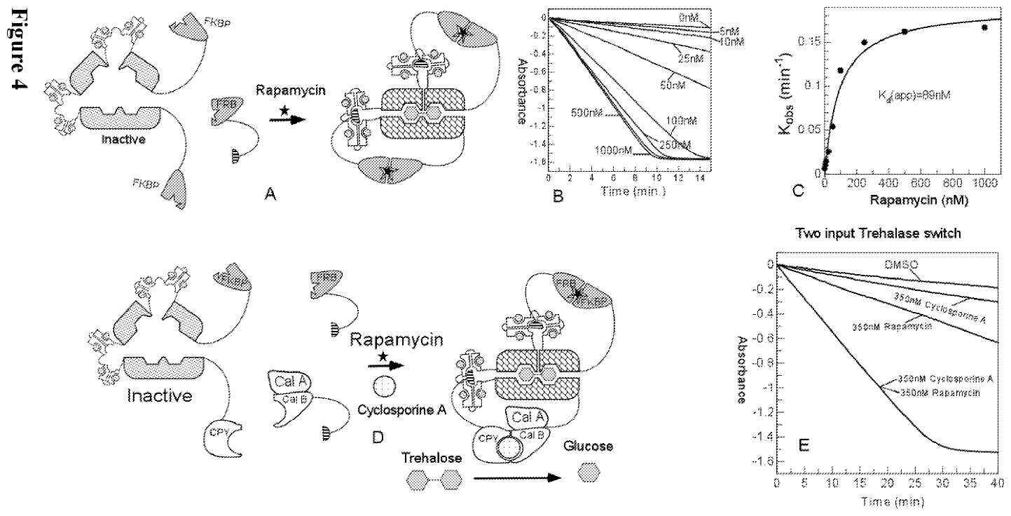

The ability of this 2CaM-Tre switch to operate in the context of a larger biosensor system was tested. According, the N- and C-termini of 2CaM-Tre were each fused to an FKBP domain, which is capable of forming a ternary complex with the target molecule rapamycin and an FRB domain (FIG. 4A). When the FKBP-2CaM-Tre-FKBP fusion protein was mixed with a fusion of FRB and a low affinity CaM-BP developed in previous studies [4], the resulting system demonstrated rapamycin-dependent activity and a large dynamic range (FIGS. 4B,C). This experiment demonstrates that developed AND-gate reporter protein constructs can be incorporated into higher order biosensor signalling systems.

Further biosensors were constructed to test whether individual CaM proteins comprised in a 2CaM-Tre switch could each be operated by different inputs. To this end, a variant biosensor was constructed, wherein a Cyclophilin (CPY) domain was attached at the C-terminus of the FKBP-2CaM-Tre fusion to generate a FKBP-2CaM-Tre-CPY fusion protein. Such a construct was able to associate with FRB-CaM-BP in a rapamycin-dependent manner, and with a fusion polypeptide containing calcineurin A and B (CalA/B) and CaM-BP (CalA/B-CaM-BP) in a cyclosporine A-dependant manner (FIG. 4D). As shown in FIG. 4E, addition of either of the ligands, rapamycin or cyclosporine A, to the mixture of FKBP-2CaM-Tre-CPY, FRB-CaM-BP and Cal A/B-CaM-BP resulted in mild activation of trehalase activity. However, addition of both ligands simultaneously led to full activation of the system (FIGS. 4D, E).

### Example 2—GDH-Based AND-gate Biosensors

A range of insertion mutants of the enzyme glucose dehydrogenase (GDH) and calmodulin were constructed. GDH catalyses the conversion of glucose to gluconolactone with the production of electrons. The activity of GDH was monitored using 5-methylphenazinium methyl sulfate (PMS) as an electron mediator and 2,6-dichlorophenolindophenol (DCPIP) as a reporter dye, which upon reduction changes from blue to colourless. The GDH structure (PDB: 1c9u) was analysed and a library of CaM domain insertions was generated. The regulatory calmodulin domain was placed at positions in the enzyme sequence where it was hypothesised that such insertions would be less likely to perturb the overall fold of the enzyme. The resulting chimeric proteins were expressed in

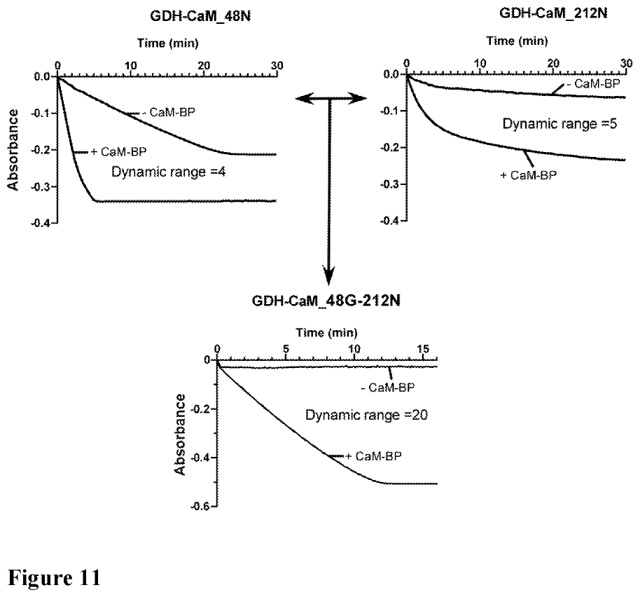

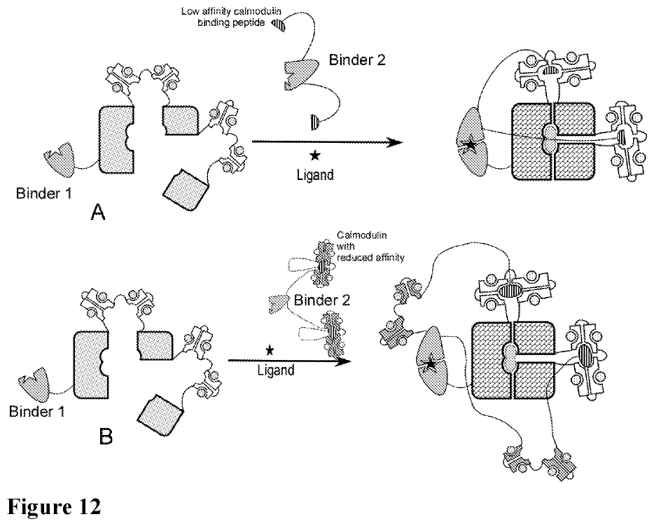

E.coli and purified to homogeneity. The activity of the chimeric proteins was assayed using the DCPIP assay. The activity of the wild type GDH as well as the GDH-CaM chimeras was monitored in the presence or absence of CaM-BP (M13). The single insertion mutants GDH-CaM_48N and GDH-CaM_212N showed CaM-BP dependant activity with rapid activation times and modest dynamic ranges of about 4 or 5 fold, respectively (FIG. 11A,B). A chimeric enzyme combining a first CaM insertion at position 48 and a second CaM insertion at position 212 was constructed. As shown in FIG. 11C, this chimera displayed an enhanced dynamic range of over 20 fold, bound CaM-BP saturably and dose dependently, and exhibited clinically useful activation times.

### Example 3—β-Lactamase-Based AND-Gate Biosensors

### BLA-CaM

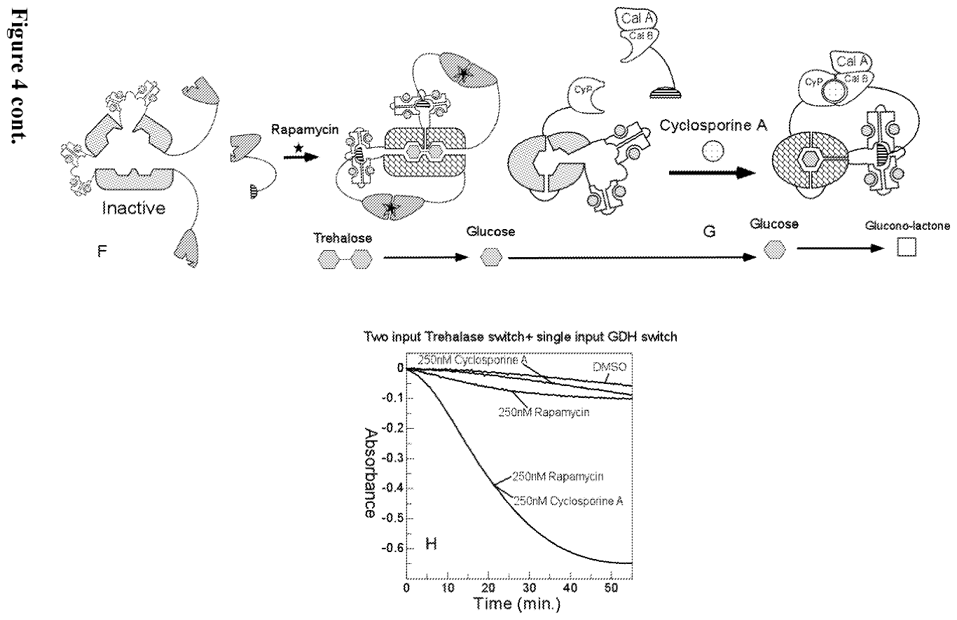

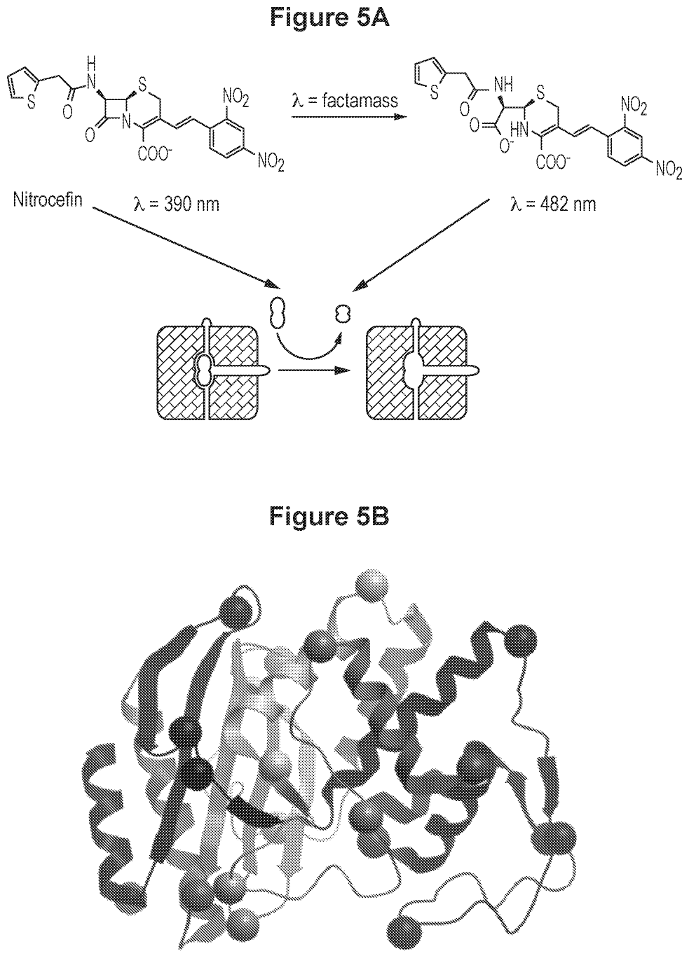

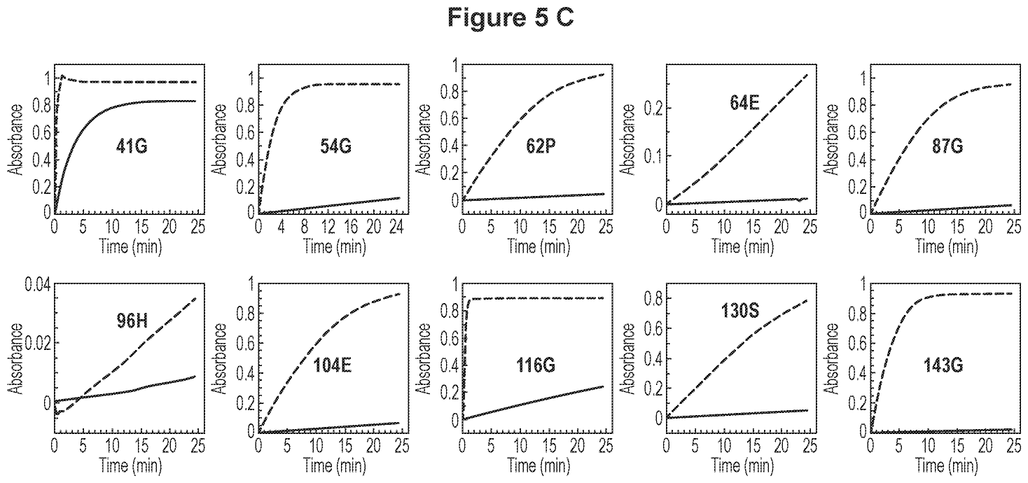

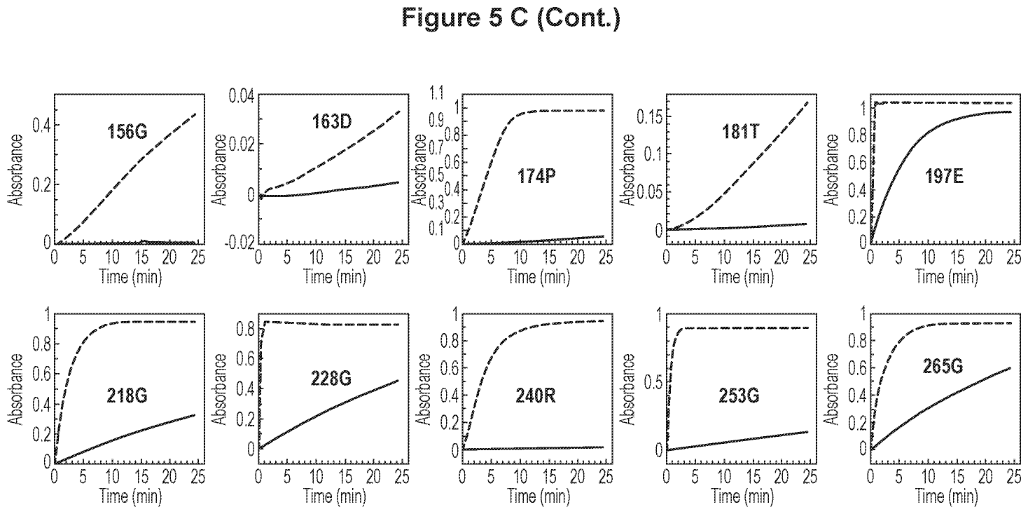

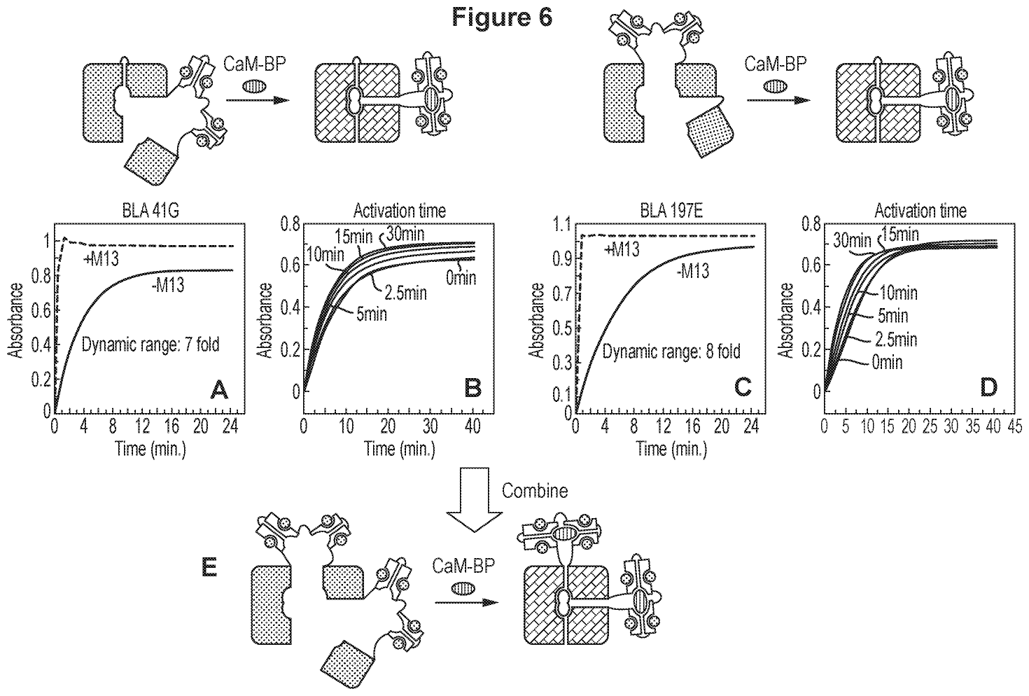

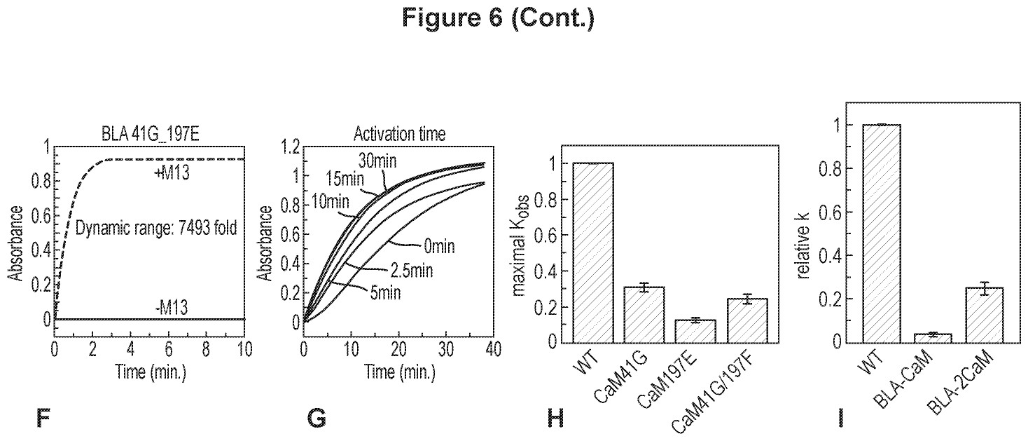

A range of insertion mutants of the enzyme β-lactamase (BLA) and calmodulin were constructed. β-lactamase degrades the beta-lactam ring in nitrocefin, which can be detected by a colour change from yellow to red (FIG. 5A). The β-lactamase structure (PDB: 3gmw) was analysed and a range of Calmodulin (BLA-CaM) chimeras were designed. The regulatory calmodulin domain was again placed at positions in the enzyme sequence where it was hypothesised that such insertions would be less likely to perturb the overall fold of the enzyme (FIG. 5B). The resulting chimeric proteins were expressed in E.coli and purified to homogeneity. The activity of the chimeric proteins was assayed using the nitrocefin assay. The activity of the wild type β-lactamase as well as the BLA-CaM chimeras was monitored in the presence or absence of CaM-BP (M13). Twenty (20) different insertion sites were analysed, as shown in FIG. 5C. For construction of a double switch variant the single insertion mutants BLA-CaM 41G and BLA-CaM 197E, which showed CaM-BP dependant activity with rapid activation times and modest dynamic ranges of about 7 or 8 fold, respectively (FIGS. 6A-D), were selected. A chimeric enzyme combining a first CaM insertion at position 41 and a second CaM insertion at position 197 was constructed (FIG. 6E). As shown in FIG. 5F and 5G, this chimera displayed a remarkable dynamic range of over 7000 fold, bound CaM-BP saturably and dose dependently, and exhibited clinically useful activation times.

Analysis of the catalytic activity of the developed switch modules demonstrates only a modest decrease in the activity of the double insertion chimera, as compared to the parental single switch enzymes. The double insertion variant retained about 25% of the activity observed for the wild type enzyme (FIG. 6H). Importantly, the double insertion mutant exhibited a significantly higher maximal activity, as compared to the best-performing single mutant (CaM-BLA 253G chimera), which retained less than 5% of the maximal activity, as compared to the wild type enzyme (FIG. 6I).

### FKBP-2CaM-BLA-FKBP & FRB-CaM-BP

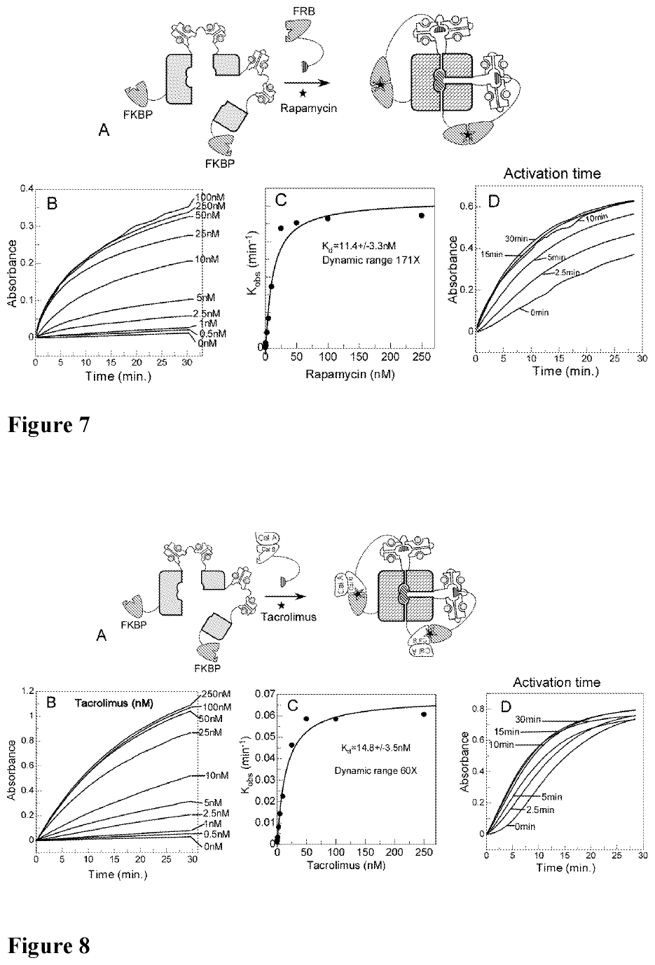

To test the utility of the developed 2CaM-BLA switch module, FKBP domains were furnished on both the N- and C-termini (FKBP-2CaM-BLA-FKBP), and its activity in the presence or absence of rapamycin and a fusion between an FRB domain and a truncated calmodulin binding peptide (FRB-CaM-BP) was tested (FIG. 7A). Addition of rapamycin to a solution of FKBP-2CaM-BLA-FKBP and FRB-CaM-BP resulted in a dose dependent increase of the β-lactamase activity (FIG. 7B). Fit of the data led to an apparent K_{d} of 11 nM and a dynamic range of 171 fold (FIG. 7C). The biosensor fully activated within 10 minutes (FIG. 7D).

### FKBP-2CaM-BLA-FKBP & Calcineurin A/B-CaM-BP

The FRB domain was then replaced with a fusion between Calcineurin A and B, to form a Calcineurin A/B-CaM-BP fusion (FIG. 8A), and the response of a mixture of FKBP-2CaM-BLA-FKBP and Calcineurin A/B-CaM-BP to tacrolimus was tested. Addition of tacrolimus to the mixture resulted in a dose dependent increase of the β-lactamase activity (FIG. 8B). Fit of the data led to an apparent K_{d} of 14 nM and a dynamic range of 60 fold (FIG. 8C). The biosensor fully activated within 10 minutes (FIG. 8D).

MTX Biosensor (VHH-2CaM-BLA-VHH & nanoCLAMP-CaM-BP)

To test the developed biosensor platform on small molecule analytes with different structure we constructed a biosensor of methotrexate (MTX) by fusing 2CaM-BLA to a VHH binder of methotrexate (PDB: 3qxv). A nanoCLAMP domain capable of recognising the

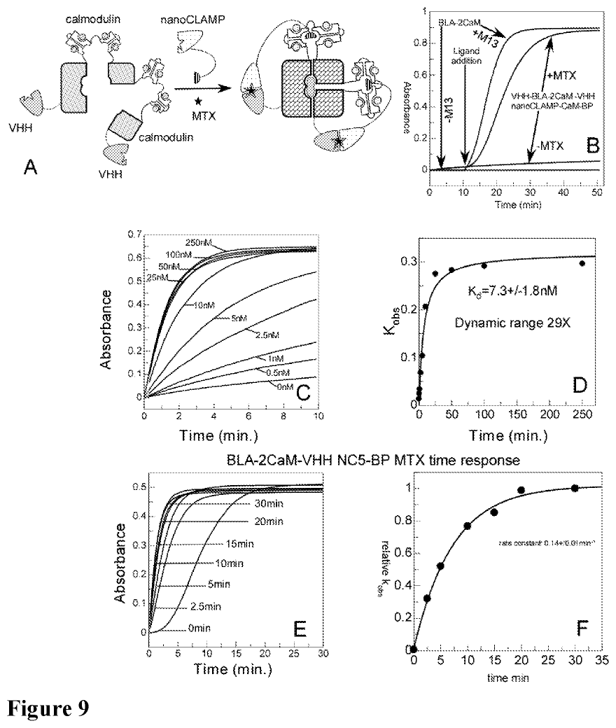

VHH: MTX complex, was then fused to the low affinity calmodulin binding peptide. Addition of MTX to the mixture of VHH-2CaM-BLA-VHH and nanoCLAMP-CaM-BP resulted in rapid increase of β-lactamase activity (FIG. 9B) achieving a comparable maximal activity when VHH-2CaM-BLA-VHH was activated by the addition of M13 binding peptide. The response was dose dependent (FIG. 9C) and the apparent K_{d} value was 7.3 nM, which is close to the affinity of the VHH: MTX interaction (FIG. 9D). The biosensor achieved half maximal activation in 5 minutes and full activation in 15 minutes (FIGS. 9E-F).

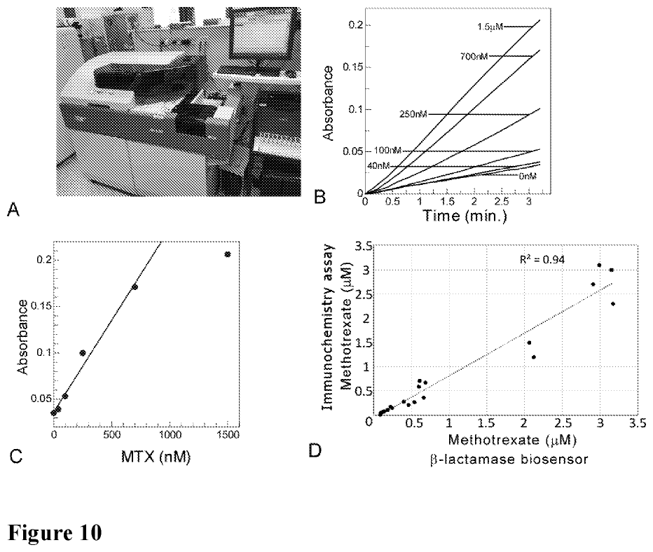

The utility of the developed test was validated by porting the assay onto the Beckman AU-480 clinical chemistry analyser (FIG. 10A) where the VHH-2CaM-BLA-VHH-based assay demonstrated the ability to quantify MTX in human serum (FIGS. 10 B,C).

### Thermostable MTX Biosensor

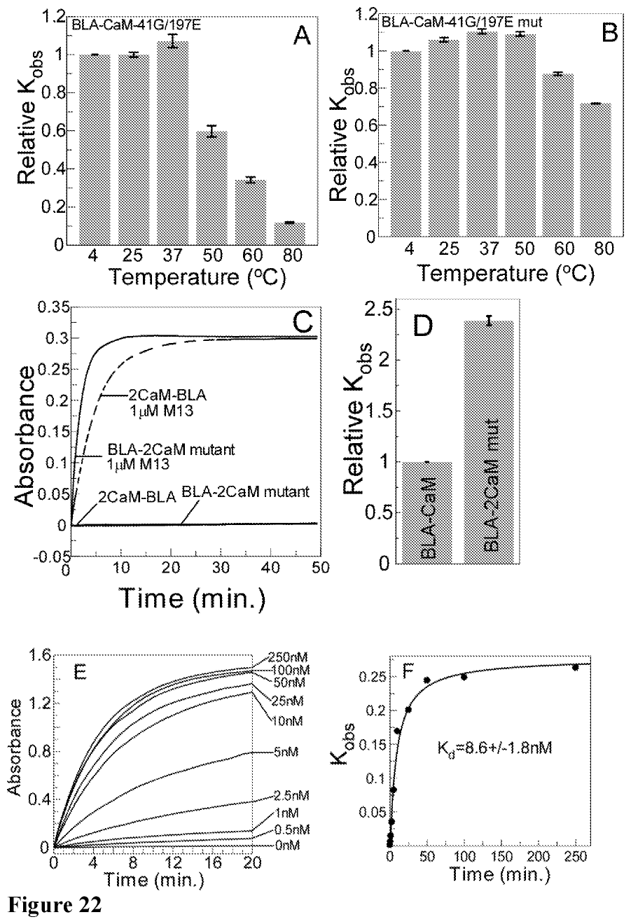

To test the MTX biosensor platform was workable across various β-lactamase-calmodulin variant sequences, a thermostabilized β-lactamase-Calmodulin chimera MTX biosensor was constructed. The biosensor was constructed by constructing a 2CaM-BLA insertion mutant (SEQ ID NO: 86) from a thermostable BLA comprising K55Q, S82A, G92D, T140K, H153R, V184A mutations (TEM-1 BLA, SEQ ID NO: 85). Use of the mutated TEM-1 BLA resulted in increased thermostability (FIG. 22B). This 2CaM-BLA was then fused to a VHH binder of methotrexate (PDB: 3qxv) to create the thermostable VHH-2CaM-BLA-VHH biosensor (SEQ ID NO: 87, also referred to as 2VHH 2CaM-BLA-41G 197E). A nanoCLAMP domain capable of recognising the VHH: MTX complex, was then fused to the low affinity calmodulin binding peptide. Addition of MTX to the mixture of VHH-2CaM-BLA-VHH and nanoCLAMP-CaM-BP resulted in rapid increase of β-lactamase activity (FIG. 22F).

### Example 4—Signalling Cascades Comprising AND-Gate Biosensors

The presented approach can be applied to more than a single enzyme, enabling regulation of enzymatic and signalling cascades.

### Trehalose to Glucose by Rapamycin Input; Glucose to Gluconolactone by Cyclosporine A Input

To illustrate the application of the biosensors in such a system, the two-input switch module shown in FIG. 4F was combined with a CaM-GDH biosensor responsive to cyclosporine A, as described previously [4] (FIG. 4G). In this system, production of glucose from trehalose by the 2FKBP-Tre-2CaM and FRB-CaM-BP biosensor is regulated by rapamycin input. The subsequent conversion of glucose to gluconolactone, with the production of electrons, by the CaM-GDH-CpY and Cal A/B-CaM-BP biosensor is controlled by cyclosporine A, which regulates the GDH activity. Activity analysis of such a system revealed that while individually rapamycin and cyclosporine A produced responses close to the background, their combination led to a large increase in activity which is consistent with the idea of pathway regulation at multiple nodes.

### Trehalose to Glucose by Rapamycin Input; Glucose to Gluconolactone by MTX Input

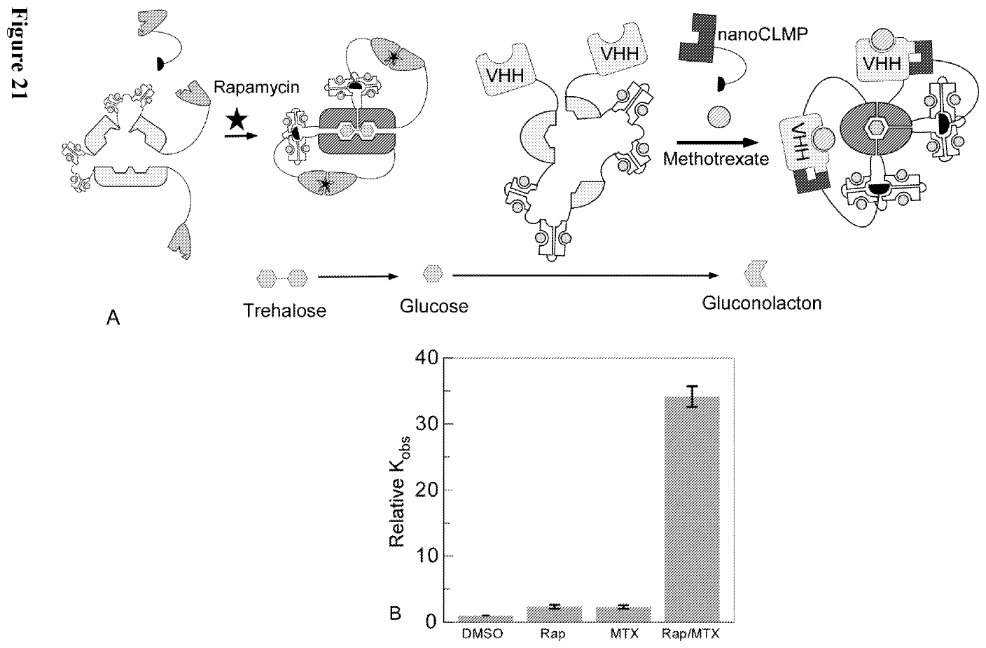

To illustrate the application of multiple two-input switches in a cascade, the two-input switch shown in FIG. 4F was combined with a CaM-GDH biosensor responsive to MTX (FIG. 21A). In this system, production of glucose from trehalose by the FKBP-2CaM-Tre-CpY and FRB-CaM-BP biosensor is regulated by rapamycin input. The subsequent conversion of glucose to gluconolactone, with the production of electrons, by the 2CaM-GDH-2VHH (which could also be referred to as VHH-2CaM-GDH-VHH) and nanoCLAMP-CaM-BP biosensor is controlled by MTX, which regulates the GDH activity. Activity analysis of such a system revealed that while individually rapamycin and MTX produced responses close to the background, their combination led to a large increase in activity (FIG. 21B) which is consistent with the idea of pathway regulation at multiple nodes.

### REFERENCES

(1) Wodak, S. J.; et al., Allostery in Its Many Disguises: From Theory to Applications. Structure 2019, 27 (4), 566-578.
(2) Clark, J. J.; et al., Inherent versus Induced Protein Flexibility: Comparisons within and between Apo and Holo Structures. PLOS Comput. Biol. 2019, 15 (1), 1-21.
(3) Stein, V.; Alexandrov, K. Protease-Based Synthetic Sensing and Signal Amplification. Proc. Natl. Acad. Sci. U. S. A. 2014, 111 (45), 15934-15939.
(4) Guo, Z.; et al., Generalizable Protein Biosensors Based on Synthetic Switch Modules. J. Am. Chem. Soc. 2019, 141 (20), 8128-8135.
(5) Nadler, D. C.; et al., Rapid Construction of Metabolite Biosensors Using Domain-Insertion Profiling. Nat. Commun. 2016, 7, 12266.
(6) Merithew, E.; et al., Structural Plasticity of an Invariant Hydrophobic Triad in the Switch Regions of Rab GTPases Is a Determinant of Effector Recognition. J Biol.Chem. 2001, 276 (17), 13982-13988.
(7) Guntas, G.; et al., Directed Evolution of Protein Switches and Their Application to the Creation of Ligand-Binding Proteins. Proc Natl Acad Sci U S A 2005, 102 (32), 11224-11229.

1. A reporter protein comprising a first heterologous amino acid sequence which is responsive to binding of a first regulator moiety and a second heterologous amino acid sequence which is responsive to binding of a second regulator moiety, wherein binding of the first regulator moiety to the first heterologous amino acid sequence and binding of the second regulator moiety to the second heterologous amino acid sequence, reversibly regulates the activity of the reporter protein.

2. The reporter protein of claim 1, wherein the reporter protein is an enzyme and wherein the activity of the reporter protein is the catalytic activity of the enzyme; or wherein the reporter protein is a fluorescent protein and wherein the activity of the reporter protein is the fluorescence of the fluorescent protein.

3. The reporter protein of claim 1, wherein:
(a) binding of the first regulator moiety to the first heterologous amino acid sequence and binding of the second regulator moiety to the second heterologous amino acid sequence, reversibly activates the activity of the reporter protein; and/or
(b) the dynamic range of the reporter protein is at least 10 fold, optionally at least 20 fold; and/or
(c) the first heterologous amino acid sequence is provided as an insert within the amino acid sequence of the reporter protein and the second heterologous amino acid sequence is provided as an insert within the amino acid sequence of the reporter protein.

4. (canceled)

5. (canceled)

6. The reporter protein of claim 1, wherein:
(a) the first heterologous amino acid sequence and/or the second heterologous amino acid sequence is a protein;
(b) the first heterologous amino acid sequence and the second heterologous amino acid sequence are proteins that are capable of undergoing a conformational change in response to binding of a regulator moiety;
(c) the first heterologous amino acid sequence and the second heterologous amino acid sequence are the same protein, or a functional fragment thereof;
(d) the first heterologous amino acid sequence and/or the second heterologous amino acid sequence is a calmodulin protein, or a functional fragment thereof.

7. The reporter protein of claim 1, wherein:
(a) the first regulator moiety and/or second regulator moiety is a peptide;
(b) the first regulator moiety and the second regulator moiety are the same peptide; and/or
(c) the first regulator moiety and/or second regulator moiety is a calmodulin-binding peptide.

8. The reporter protein of claim 1, wherein binding of a regulator moiety to the first heterologous amino acid sequence causes a conformational change in the structure of the first heterologous amino acid sequence and wherein binding of a regulator moiety to the second heterologous amino acid sequence causes a conformational change in the structure of the second heterologous amino acid sequence;
optionally wherein binding of a calmodulin-binding peptide to the first and second heterologous amino acid sequences which are both calmodulin, activates the activity of the reporter protein.

9. The reporter protein of claim 1, (i) wherein the reporter protein is an enzyme selected from the group consisting of trehalase, an oxidoreductase, glucose dehydrogenase, β-lactamase, aminoglycoside phosphotransferase, α-amylase, and carbonic anhydrase; or (ii) wherein the reporter protein is a fluorescent protein selected from the group consisting of GFP, Cherry, a bacterial phytochrome (BphP)-based fluorescent protein, or a cyanobacteriochrome (CBCR)-derived fluorescent protein.

10. The reporter protein of claim 1, wherein the reporter protein further comprises a first binding moiety B1′; optionally wherein the reporter protein further comprises a second binding moiety B2′.

11. The reporter protein of claim 1, wherein:
(a) the first and second regulator moieties comprise a binding moiety B1″ which is capable of interacting with a first binding moiety B1′ on the reporter protein; or
(b) the first regulator moiety comprises a binding moiety B1″ which is capable of interacting with a first binding moiety B1′ on the reporter protein; or
(c) the second regulator moiety comprises a binding moiety B2″ which is capable of interacting with a second binding moiety B2′ on the reporter protein; or
(d) the first regulator moiety comprises a binding moiety B1″ which is capable of interacting with a first binding moiety B1′ on the reporter protein and the second regulator moiety comprises a binding moiety B2″ which is capable of interacting with a second binding moiety B2′ on the reporter protein.

12. The reporter protein of claim 10, wherein:
(a) interaction of the binding moieties B1′ and B1″ regulates the activity of the reporter protein, optionally activates the activity of the reporter protein; or
(b) interaction of the binding moieties B1′ and B1″ and interaction of the binding moieties B2′ and B2″, regulates the activity of the reporter protein, optionally activates the activity of the reporter protein; or
(c) activity of the reporter protein is activated only upon interaction of both the binding moieties B1′ and B1″ and interaction of the binding moieties B2′ and B2″.

13. The reporter protein of claim 10, wherein:
(a) binding moieties B1′ and B1″ are capable of directly binding to each other and/or binding moieties B2′ and B2″ are capable of directly binding to each other; or
(b) interaction of the binding moieties B1′ and B1″ is dependent on the presence of a first target molecule TM1; and/or interaction of the binding moieties B2′ and B2″ is dependent on the presence of a second target molecule TM2.

14. The reporter protein of claim 10, wherein interaction of the binding moieties B1′ and B1″ and interaction of the binding moieties B2′ and B2″ is dependent on the presence of both a first target molecule TM1 and a second target molecule TM2, such that activity of the reporter protein is activated only in the presence of both of the target molecules TM1 and TM2.

15. The reporter protein of claim 10, wherein:
(a) binding moieties B1′ and B1″ and binding moieties B2′ and B2″ are the same pair of binding moieties or are different pairs of binding moieties; and/or
(b) the target molecules TM1 and TM2 are the same or different.

16. The reporter protein of claim 10, wherein the presence of a first target molecule TM1 and a second target molecule TM2 reversibly regulates activity of the reporter protein, optionally activates the activity of the reporter protein.

17. A composition of matter comprising:
(A) a biosensor comprising:
(a) the reporter protein of claim 1;
(b) a first regulator moiety as defined in claim 1; and
(c) a second regulator moiety as defined in claim 1; or
(B) a composition or kit comprising the reporter protein of claim 1; or
(C) a composition or kit comprising a biosensor comprising:
(a) the reporter protein of claim 1;
(b) a first regulator moiety as defined in claim 1; and
(c) a second regulator moiety as defined in claim 1; or
(D) one or more nucleic acids encoding the reporter protein of claim 1 or the biosensor of (A); or
(E) one or more expression vectors comprising said one or more nucleic acids of (D) operably linked to one or more promoters.

18. (canceled)

19. The composition of matter of claim 17:
(a) wherein the reporter protein is an enzyme, further comprising a substrate for the enzyme; and/or
(b) further comprising target molecule TM1; and/or further comprising target molecule TM2; optionally wherein target molecule TM1 is the same as target molecule TM2; or wherein target molecule TM1 is different from target molecule TM2; and/or
(c) further comprising a biological sample, wherein the biological sample may or may not comprise target molecule TM1 and/or target molecule TM2; and/or
(d) wherein the reporter protein is an enzyme, further comprising a second enzyme comprising a heterologous amino acid sequence which is responsive to a peptide P, wherein binding of the peptide P to the heterologous amino acid sequence reversibly regulates catalytic activity of the second enzyme, wherein the substrate of the second enzyme is the catalytic product of the enzyme of the reporter protein comprising the first heterologous amino acid sequence which is responsive to binding of the first regulator moiety and the second heterologous amino acid sequence which is responsive to binding of the second regulator moiety, wherein binding of the first regulator moiety to the first heterologous amino acid sequence and binding of the second regulator moiety to the second heterologous amino acid sequence, reversibly regulates the activity of the reporter protein; optionally wherein (a) the second enzyme is an oxidoreductase, optionally glucose dehydrogenase, (b) the heterologous amino acid sequence is calmodulin or a functional fragment thereof, and (c) peptide P is a calmodulin-binding peptide.

20. A method of:
(A) detecting one or more target molecules, comprising contacting the reporter protein of claim 1 with a sample under conditions suitable for detection of the presence or absence of the one or more target molecules in the sample;
optionally wherein:
(a) the one or more target molecules comprises or consist essentially of target molecule TM1 and target molecule TM2; and/or
(b) target molecule TM1 and target molecule TM2 are the same or different; and/or
(c) the one or more target molecules is selected from the group consisting of: methotrexate, phenolic glucosides, Rapamycin, tacrolimus, and Cyclosporine A; or
(B) diagnosis of a disease or condition in an organism, comprising contacting the reporter protein of claim 1, with a sample obtained from the organism under conditions suitable for detection of the presence or absence of the one or more target molecules in the sample, wherein presence or absence of the one or more target molecules in the sample is indicative of whether the organism has, or is at risk of having, said disease or condition; or
(C) monitoring one or more target molecules in an organism, the method comprising contacting the reporter protein of claim 1, with a sample obtained from the organism under conditions suitable for detecting and/or quantifying the presence or absence of the one or more target molecules in the sample; or
(D) monitoring a metabolite of interest in an organism, the method comprising expressing the reporter protein of claim 1, in said organism under conditions suitable for detecting and/or quantifying the metabolite of interest in the organism, optionally wherein the organism is a bacterial cell; or
(E) assaying for protein-protein or protein-small molecule interactions comprising contacting the reporter protein of claim 1 with a sample under conditions suitable for detection of the presence or absence of an interaction between binding moieties B1′ and B1″ and/or an interaction between binding moieties B2′ and B2″; or an interaction between binding moieties B1′ and B1″ and a target molecule TM1 and/or an interaction between binding moieties B2′ and B2″ and a target molecule TM2.

21. (canceled)

22. A detection device that comprises a cell or chamber that comprises:
(A) the reporter protein of claim 1; or
(B) a biosensor comprising
(a) the reporter protein of claim 1,
(b) a first regulator moiety as defined in claim 1, and
(c) a second regulator moiety as defined in claim 1; or
(C) a composition or kit comprising the reporter protein of claim 1; or
(D) a composition or kit comprising a biosensor comprising
(a) the reporter protein of claim 1.
(b) a first regulator moiety as defined in claim 1, and
(c) a second regulator moiety as defined in claim 1.

23. (canceled)

24. A host cell comprising:
(A) the reporter protein of claim 1; or
(B) a biosensor comprising
(a) the reporter protein of claim 1.
(b) a first regulator moiety as defined in claim 1, and
(c) a second regulator moiety as defined in claim 1; or
(C) a composition or kit comprising the reporter protein of claim 1; or
(D) a composition or kit comprising a biosensor comprising
(a) the reporter protein of claim 1.
(b) a first regulator moiety as defined in claim 1, and (c) a second regulator moiety as defined in claim 1; or
(E) one or more nucleic acids one or more nucleic acids encoding the reporter protein of claim 1 or the biosensor of (B); or
(F) one or more expression vectors comprising said one or more nucleic acids of (E) operably linked to one or more promoters.

25. A method for converting a constitutively active enzyme into a reversibly regulated enzyme whose catalytic activity is dependent on the presence of one or more target molecules, the method comprising:
(a) generating a library of single insert enzyme mutants by inserting a heterologous amino acid sequence which is responsive to binding of a regulator moiety at a number of different locations in an enzyme sequence and selecting those single insert enzyme mutants showing a change in catalytic activity on binding of the regulator moiety;
(b) generating a library of double insert enzyme mutants by inserting an additional heterologous amino acid sequence which is responsive to binding of a regulator moiety at a second site within the enzyme sequence, and selecting those double insert enzyme mutants showing a change in catalytic activity in the presence of the regulator moiety and having an increased dynamic range as compared to the corresponding single insert enzyme mutants;
optionally wherein the change in catalytic activity occurs within less than 10 minutes, optionally less than 5 minutes.


SEQUENCE LISTING (89 sequences)

SEQ ID NO: 1 - protein, 535 aa, synthetic construct
EETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTP

SEQ ID NO: 2 - protein, 549 aa, synthetic construct
DEETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPKLAAALEHHHHHH

SEQ ID NO: 3 - protein, 263 aa, synthetic construct [sequence omitted]

SEQ ID NO: 4 - protein, 278 aa, synthetic construct
DHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGKLAAALEHHHHHH

SEQ ID NO: 5 - protein, 454 aa, synthetic construct
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 6 - protein, 460 aa, synthetic construct
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKHHHHHH

SEQ ID NO: 7 - skipped

SEQ ID NO: 8 - protein, 271 aa, synthetic construct
MSHIQRETSCSRPRLNSNLDADLYGYRWARDNVGQSGATIYRLYGKPNAPELFLKHGKGSVANDVTDEMVRLNWLTAFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 9 - protein, 283 aa, synthetic construct
AHHHHHHSSGTGMSHIQRETSCSRPRLNSNLDADLYGYRWARDNVGQSGATIYRLYGKPNAPELFLKHGKGSVANDVTDEMVRLNWLTAFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 10 - protein, 246 aa, synthetic construct
MRKILISAVLVLSSISISFAEHEWSYEGEKGPEHWAQLKPEFFWCKLKNQSPINIDKKYKVKANLPKLNLYYKTAKESEVVNNGHTIQINIKEDNTLNYLGEKYQLKQFHFHTPSEHTIEKKSYPLEIHFVHKTEDGKILVVGVMAKLGKTNKELDKILNVAPAEEGEKILDKNLNLNNLIPKDKRYMTYSGSLTTPPCTEGVRWIVLKKPISISKQQLEKLKSVMVNPNNRPVQEINSRWIIEGF

SEQ ID NO: 11 - protein, 143 aa, synthetic construct
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTA

SEQ ID NO: 12 - protein, 152 aa, synthetic construct
GSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSG

SEQ ID NO: 13 - protein, 4 aa, synthetic construct
GSGG

SEQ ID NO: 14 - protein, 5 aa, synthetic construct
GGSSG

SEQ ID NO: 15 - protein, 148 aa, synthetic construct
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGFISAAELRHVMTNLGEKLTDEEVDEMIREADIDGDGQVNYEEFVTMMTSK

SEQ ID NO: 16 - protein, 143 aa, synthetic construct
TEEQIAEFKEAFSLFDKDGDGTITTKELGTVMRSLGQNPTEAELQDMINEVDADGNGTIDFPEFLTMMARKMKDTDSESEIREAFRVADKDGNGYISAAEARHVMTNLGEKLTDEEVDEMIREADIDGDGQVNYEEAVQMMTA

SEQ ID NO: 17 - protein, 26 aa, synthetic construct
KRRWKKNFIAVSAANRFKKISSSGAL

SEQ ID NO: 18 - protein, 16 aa, synthetic construct
KRRWKKNFIAVSAANR

SEQ ID NO: 19 - protein, 16 aa, synthetic construct
KRRWKKNFIAVASASA

SEQ ID NO: 20 - protein, 21 aa, synthetic construct
KRRWKKNFIAVSAANRFKKIS

SEQ ID NO: 21 - protein, 10 aa, synthetic construct
KRRWKKNFIA

SEQ ID NO: 22 - protein, 33 aa, synthetic construct
EVTVGKFYATFLIQEYFRKFKKRKEQGLVGKPS

SEQ ID NO: 23 - protein, 14 aa, synthetic construct
EVTVGKFYATFLIQ

SEQ ID NO: 24 - protein, 22 aa, synthetic construct
HMGKIYAAMMIMEYYRQSKAKK

SEQ ID NO: 25 - protein, 22 aa, synthetic construct
HMGKIYAAMMIMEAYRQSKAKK

SEQ ID NO: 26 - protein, 22 aa, synthetic construct
EMRQKIRSHAHALLAANRFMDM

SEQ ID NO: 27 - protein, 30 aa, synthetic construct
NMGQHLDVKLVPSSSYIKVVKAFHHSSLHE

SEQ ID NO: 28 - protein, 15 aa, synthetic construct
GRNWKNFALVPLLRD

SEQ ID NO: 29 - protein, 40 aa, synthetic construct
LTEIKKQIPQKEWDKVVNTPNSLEKQKGVTNLLIKYGIER

SEQ ID NO: 30 - protein, 15 aa, synthetic construct
IRNKIRAIGKMARVF

SEQ ID NO: 31 - protein, 20 aa, synthetic construct
HSMQALSWRKLYLSRAKLKA

SEQ ID NO: 32 - protein, 22 aa, synthetic construct
ADKLRAACIRIQKTIRGWLLRK

SEQ ID NO: 33 - protein, 28 aa, synthetic construct
MQRAAITVQRYVRGYQARCYAKFLRRTK

SEQ ID NO: 34 - protein, 43 aa, synthetic construct
EIRKILRNNLQKTRQRLRSYNRHTLVADPYEEAWNQMLLRRQK

SEQ ID NO: 35 - protein, 23 aa, synthetic construct
RLSFKTVALLVLACVRMKRIAFY

SEQ ID NO: 36 - protein, 106 aa, synthetic construct
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 37 - protein, 95 aa, synthetic construct
RVAILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 38 - protein, 382 aa, synthetic construct
TSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEED

SEQ ID NO: 39 - protein, 168 aa, synthetic construct
NEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 40 - protein, 559 aa, synthetic construct
TSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGSGSGSGGGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 41 - protein, 52 aa, synthetic construct
AVDANSLAEAKVLANRELDKYGVSDFYKRLINKAKTVEGVEALKLHILAALP

SEQ ID NO: 42 - protein, 165 aa, synthetic construct
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 43 - protein, 74 aa, synthetic construct
DTTVSEPAPSCVTLYQSWRYSQADNGCAETVTVKVVYEDDTEGLCYAVAPGQITTVGDGYIGSHGHARYLARCL

SEQ ID NO: 44 - protein, 121 aa, synthetic construct
QVQLVESGGGTVPAGGSLRLSCAASGNTLCTYDMSWYRRAPGKGRDFVSGIDNDGTTTYVDSVAGRFTISQGNAKNTAYLQMDSLKPDDTAMYYCKPSLRYGLPGCPIIPWGQGTQVTVSS

SEQ ID NO: 45 - protein, 118 aa, synthetic construct
QVQLQESGGGLVQAGGSLRLSCAASGYISDAYYMGWYRQAPGKEREFVATITHGTNTYYADSVKGRFTISRDNAKNTVYLQMNSLKPEDTAVYYCAVLETRSYSFRYWGQGTQVTVSS

SEQ ID NO: 46 - protein, 126 aa, synthetic construct
GSQVQLVESGGGLVQAGGSLRLSCAASRRSSRSWAMAWFRQAPGKEREFVAKISGDGRLTTYGDSVKGRFTISRDNAEYLVYLQMDSLKPEDTAVYYCAADDNYVTASWRSGPDYWGQGTQVTVSS

SEQ ID NO: 47 - protein, 140 aa, synthetic construct
NPSLIRSESWIPYFGNEANLLDGDDNTGVWYLKDSYEFSLAGEFIGLDLGKEIKLDGIRFVIGKNGGGSSDKWNKFKLEYSLDNESWTTIKEYDKTGAPAGKDVIEESFETPISAKYIRLTNLEQIFHALTFSEFAIVSD

SEQ ID NO: 48 - protein, 205 aa, synthetic construct
EDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPG

SEQ ID NO: 49 - protein, 57 aa, synthetic construct
AEYVRALFDFNGNDEEDLPFKKGDILRIRDKPEEQWWNAEDSEGKRGMIPVPYVEKY

SEQ ID NO: 50 - protein, 11 aa, synthetic construct
PPPPLPPKRRR

SEQ ID NO: 51 - protein, 851 aa, synthetic construct
DEETPVTPQPPDILLGPLFNDVQNAKLFPDQKTFADAVPNSDPLMILADYRMQQNQSGFDLRHFVNVNFTLPKEGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPKLAAALEHHHHHH

SEQ ID NO: 52 - protein, 851 aa, synthetic construct
DEETPVTPQPPDILLGPLFNDVQNAKLFPDQKTFADAVPNSDPLMILADYRMQQNQSGFDLRHFVNVNFTLPKEGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPKLAAALEHHHHHH

SEQ ID NO: 53 - protein, 1086 aa, synthetic construct
DVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGEETPVTPQPPDILLGPLFNDVQNAKLFPDQKTFADAVPNSDPLMILADYRMQQNQSGFDLRHFVNVNFTLPKEGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGGSGGSGSGSGGSGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEKLAAALEHHHHHH

SEQ ID NO: 54 - protein, 1030 aa, synthetic construct
EETPVTPQPPDILLGPLFNDVQNAKLFPDQKTFADAVPNSDPLMILADYRMQQNQSGFDLRHFVNVNFTLPKEGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGGSGGSGSGSGGSGGMVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLEKLAAALEHHHHHH

SEQ ID NO: 55 - protein, 429 aa, synthetic construct
DHPETLVKVKDAEDQLGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGKLAAALEHHHHHH

SEQ ID NO: 56 - protein, 429 aa, synthetic construct
DHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGLLTLASRQQLIDWMEADKVAGPLLRSALPAGWFIADKSGAGERGSRGIIAALGPDGKPSRIVVIYTTGSQATMDERNRQIAEIGASLIKHWGKLAAALEHHHHHH

SEQ ID NO: 57 - protein, 579 aa, synthetic construct
DHPETLVKVKDAEDQLGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGLLTLASRQQLIDWMEADKVAGPLLRSALPAGWFIADKSGAGERGSRGIIAALGPDGKPSRIVVIYTTGSQATMDERNRQIAEIGASLIKHWKLAAALEHHHHHH

SEQ ID NO: 58 - protein, 839 aa, synthetic construct
DVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGGGSGGSGGGGSGGSGGHPETLVKVKDAEDQLGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGLLTLASRQQLIDWMEADKVAGPLLRSALPAGWFIADKSGAGERGSRGIIAALGPDGKPSRIVVIYTTGSQATMDERNRQIAEIGASLIKHWGGSGGSGSGSGGSGGGSGSGGSGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEKLAAALEHHHHHH

SEQ ID NO: 59 - protein, 852 aa, synthetic construct
DGSQVQLVESGGGLVQAGGSLRLSCAASRRSSRSWAMAWFRQAPGKEREFVAKISGDGRLTTYGDSVKGRFTISRDNAEYLVYLQMDSLKPEDTAVYYCAADDNYVTASWRSGPDYWGQGTQVTVSSGGSGGGGSGGSGGHPETLVKVKDAEDQLGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGLLTLASRQQLIDWMEADKVAGPLLRSALPAGWFIADKSGAGERGSRGIIAALGPDGKPSRIVVIYTTGSQATMDERNRQIAEIGASLIKHWGGSGGSGGGSQVQLVESGGGLVQAGGSLRLSCAASRRSSRSWAMAWFRQAPGKEREFVAKISGDGRLTTYGDSVKGRFTISRDNAEYLVYLQMDSLKPEDTAVYYCAADDNYVTASWRSGPDYWGQGTQVTVSSKLAAALEHHHHHH

SEQ ID NO: 60 - protein, 618 aa, synthetic construct
DVPLIPSQFAKAKSENFDKKVILSNLNKPHALLWGPDNQIWLTERATGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKLAAALEHHHHHH

SEQ ID NO: 61 - protein, 618 aa, synthetic construct
DVPLIPSQFAKAKSENFDKKVILSNLNKPHALLWGPDNQIWLTERATNKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKLAAALEHHHHHH

SEQ ID NO: 62 - protein, 769 aa, synthetic construct
DVPLIPSQFAKAKSENFDKKVILSNLNKPHALLWGPDNQIWLTERATGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKLAAALEHHHHHH

SEQ ID NO: 63 - protein, 618 aa, synthetic construct
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSGCGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSCGNRYRDVIASPDGNVLYVLTDTAGNVQKDDGSVTNTLENPGSLIKFTYKAKKLAAALEHHHHHH

SEQ ID NO: 64 - protein, 798 aa, synthetic construct
DVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSGCGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSCGNRYRDVIASPDGNVLYVLTDTAGNVQKDDGSVTNTLENPGSLIKFTYKAKGGSGGSGSGSGGSGGMVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLEKLAAALEHHHHHH

SEQ ID NO: 65 - protein, 137 aa, synthetic construct
AHHHHHHSSGTRVAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGSGSGGSGGKRRWKKNFIAVSAANR

SEQ ID NO: 66 - protein, 724 aa, synthetic construct
DVPLIPSQFAKAKSENFDKKGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGSGSGGAHHHHHHSSGTSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGSGSGSGGGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDVGGSGGSGSGSGGSGGKRRWKKNFIAVSAANR

SEQ ID NO: 67 - protein, 190 aa, synthetic construct
DGSSHHHHHHNPSLIRSESWIPYFGNEANLLDGDDNTGVWYLKDSYEFSLAGEFIGLDLGKEIKLDGIRFVIGKNGGGSSDKWNKFKLEYSLDNESWTTIKEYDKTGAPAGKDVIEESFETPISAKYIRLTNLEQIFHALTFSEFAIVSDGGSGSGGSGGGSGSSGGSGGSGGGKRRWKKNFIAVSAANR

SEQ ID NO: 68 - DNA, 4533 bp, synthetic construct
  source 1..4533: mol_type = other DNA
ggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacgaagatcctttgatcttttctacggggtctgacgctcagtggaacgaaaactcacgttaagggattttggtcatgacctatttgtttatttttctaaatacattcaaatatgtatccgctcatgagacaataaccctgataaatgcttcaatcatgattgaaaaaggaagagtccatggataaacatttattggctaaaattgctttattaggcgctgctcagctagttacactctcagcatttgctgatggatcccatccggagacattagtaaaagtaaaagacgccgaagatcaactgggggctcgggttggctacattgagcttgatcttaatagcgggaagatacttgaaagttttcgtcccgaggaacgttttccgatgatgtcaacttttaaggtactgctttgtggtgccgttctgagccgtgtagacgcgggtcaagaacagttgggtagaagaattcactattcacagaatgacctggtggaatactcccccgttacggaaaagcatttgacagacgggatgacggtgcgggaactgtgctcagcggcgattactatgagcgacaacaccgccgcaaacttgttgctgacgacgattggtggtcctaaagaattaacggcctttctgcacaatatgggggatcacgtcacacggctggatcggtgggaaccggagctgaatgaagctatacctaacgatgagagagacaccacaatgcccgtggctatggcgactacattacggaagctgttgacaggagagttacttacactggcgtctagacagcagttaatcgactggatggaagctgataaggtagccgggccattgttacggagcgcgttacctgcggggtggtttatcgcggataagagcggagcgggggaacggggttctagagggatcattgctgctttgggaccggacggcagcggagggacagaggaacaaatcgccgaatttaaggaagccttttcgttatttgacaaggacggagacgggactattacgacgaaggagcttggcaccgtcatgcgttccctgggtcagaatcctacagaagccgagttacaagatatgataaatgaggttgacgctgacggtaacggaactatcgacttccctgaatttttaaccatgatggcacgcaaaatgaaagatacagatagcgaggaagagatccgggaagcctttagagtgtttgacaaagacgggaacggatacatatcagccgctgagttgcggcacgttatgactaaccttggcgaaaaacttactgacgaagaagtagacgaaatgatacgtgaggctgacatcgacggagatggacaggtcaactatgaggagttcgtgcaaatgatgactgctggcggatcgagcgggaagccgagtcgcatcgtggtaatatatacaactggttctcaggccacaatggatgagcggaatagacagatagccgaaattggcgctagcttaataaagcactggtaagcttgcggccgcataatgcttaagtcgaacagaaagtaatcgtattgtacacggccgcataatcgaaataactcacgttaagggattttggtcatgacctatttgtttatttttctaaatacattcaaatatgtatccgctcatgagacaataaccctgataaatgcttcaatcatgattgaaaaaggaagagtcatatggacaagcacttgttagcaaagattgcattattgggggcagcacaactcgtaactctaagtgctttcgcagacagatctcaattggatatcggccggccacgcgatcgctgacgtcggtaccggaggtagtggctccggtggttcaggtggcggtagcggtagctccggaggaagtggcggatctgggggtggaaagcgtcgttggaaaaagaactttatagccgtgagtgctgccaatcggtagctcgagcctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgtt

SEQ ID NO: 69 - protein, 441 aa, synthetic construct
MDKHLLAKIALLGAAQLVTLSAFADGSHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGKPSRIVVIYTTGSQATMDERNRQIAEIGASLIKHW

SEQ ID NO: 70 - protein, 482 aa, synthetic construct
MDKHLLAKIALLGAAQLVTLSAFADGSHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGKPSRIVVIYTTGSQATMDERNRQIAEIGASLIKHWGGSGGSGGSGSGSGGKRRWKKNFIAVSAANRFKKISSSGAL

SEQ ID NO: 71 - DNA, 4986 bp, synthetic construct
  source 1..4986: mol_type = other DNA
ggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacgaagatcctttgatcttttctacggggtctgacgctcagtggaacgaaaactcacgttaagggattttggtcatgacctatttgtttatttttctaaatacattcaaatatgtatccgctcatgagacaataaccctgataaatgcttcaatcatgattgaaaaaggaagagtccatggataaacatttattggctaaaattgctttattaggcgctgctcagctagttacactctcagcatttgctgatggatcccatccggagacattagtaaaagtaaaagacgccgaagatcaactgggcagcggagggacagaggaacaaatcgccgaatttaaggaagccttttcgttatttgacaaggacggagacgggactattacgacgaaggagcttggcaccgtcatgcgttccctgggtcagaatcctacagaagccgagttacaagatatgataaatgaggttgacgctgacggtaacggaactatcgacttccctgaatttttaaccatgatggcacgcaaaatgaaagatacagatagcgaggaagagatccgggaagcctttagagtgtttgacaaagacgggaacggatacatatcagccgctgagttgcggcacgttatgactaaccttggcgaaaaacttactgacgaagaagtagacgaaatgatacgtgaggctgacatcgacggagatggacaggtcaactatgaggagttcgtgcaaatgatgactgctggcggatcgagcggggctcgggttggctacattgagcttgatcttaatagcgggaagatacttgaaagttttcgtcccgaggaacgttttccgatgatgtcaacttttaaggtactgctttgtggtgccgttctgagccgtgtagacgcgggtcaagaacagttgggtagaagaattcactattcacagaatgacctggtggaatactcccccgttacggaaaagcatttgacagacgggatgacggtgcgggaactgtgctcagcggcgattactatgagcgacaacaccgccgcaaacttgttgctgacgacgattggtggtcctaaagaattaacggcctttctgcacaatatgggggatcacgtcacacggctggatcggtgggaaccggagctgaatgaagctatacctaacgatgagagagacaccacaatgcccgtggctatggcgactacattacggaagctgttgacaggagggtcaggtggcactgaagagcagatcgcggagttcaaagaggctttctccttattcgataaagatggtgatggcacaattaccaccaaagaacttgggacggtgatgcgttcgctgggacaaaaccctactgaggcggaattacaggacatgataaacgaagtagatgcagatggaaatggtacaatcgattttcctgagttcttaacgatgatggctcgcaagatgaaggacactgactcagaagaggaaatccgggaggcgttcagagtcttcgataaggatggcaatggttatataagcgcggcagaattgcggcatgtaatgacaaatcttggagagaaacttacagatgaggaggttgatgagatgatacgtgaagcagatatcgatggtgacggtcaagtgaattacgaagaatttgtccagatgatgacagcagggggtagctcaggcttacttacactggcgtctagacagcagttaatcgactggatggaagctgataaggtagccgggccattgttacggagcgcgttacctgcggggtggtttatcgcggataagagcggagcgggggaacggggttctagagggatcattgctgctttgggaccggacggaaagccgagtcgcatcgtggtaatatatacaactggttctcaggccacaatggatgagcggaatagacagatagccgaaattggcgctagcttaataaagcactggtaagcttgcggccgcataatgcttaagtcgaacagaaagtaatcgtattgtacacggccgcataatcgaaataactcacgttaagggattttggtcatgacctatttgtttatttttctaaatacattcaaatatgtatccgctcatgagacaataaccctgataaatgcttcaatcatgattgaaaaaggaagagtcatatggacaagcacttgttagcaaagattgcattattgggggcagcacaactcgtaactctaagtgctttcgcagacagatctcaattggatatcggccggccacgcgatcgctgacgtcggtaccggaggtagtggctccggtggttcaggtggcggtagcggtagctccggaggaagtggcggatctgggggtggaaagcgtcgttggaaaaagaactttatagccgtgagtgctgccaatcggtagctcgagcctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgtt

SEQ ID NO: 72 - DNA, 7021 bp, synthetic construct
  source 1..7021: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatggataaacatttattggctaaaattgctttattaggcgctgctcagctagttacactctcagcatttgctgatcatccggagacattagtaaaagtaaaagacgccgaagatcaactgggcagcggagggacagaggaacaaatcgccgaatttaaggaagccttttcgttatttgacaaggacggagacgggactattacgacgaaggagcttggcaccgtcatgcgttccctgggtcagaatcctacagaagccgagttacaagatatgataaatgaggttgacgctgacggtaacggaactatcgacttccctgaatttttaaccatgatggcacgcaaaatgaaagatacagatagcgaggaagagatccgggaagcctttagagtgtttgacaaagacgggaacggatacatatcagccgctgagttgcggcacgttatgactaaccttggcgaaaaacttactgacgaagaagtagacgaaatgatacgtgaggctgacatcgacggagatggacaggtcaactatgaggagttcgtgcaaatgatgactgctggcggatcgagcggggctcgggttggctacattgagcttgatcttaatagcgggaagatacttgaaagttttcgtcccgaggaacgttttccgatgatgtcaacttttaaggtactgctttgtggtgccgttctgagccgtgtagacgcgggtcaagaacagttgggtagaagaattcactattcacagaatgacctggtggaatactcccccgttacggaaaagcatttgacagacgggatgacggtgcgggaactgtgctcagcggcgattactatgagcgacaacaccgccgcaaacttgttgctgacgacgattggtggtcctaaagaattaacggcctttctgcacaatatgggggatcacgtcacacggctggatcggtgggaaccggagctgaatgaagctatacctaacgatgagagagacaccacaatgcccgtggctatggcgactacattacggaagctgttgacaggagggtcaggtggcactgaagagcagatcgcggagttcaaagaggctttctccttattcgataaagatggtgatggcacaattaccaccaaagaacttgggacggtgatgcgttcgctgggacaaaaccctactgaggcggaattacaggacatgataaacgaagtagatgcagatggaaatggtacaatcgattttcctgagttcttaacgatgatggctcgcaagatgaaggacactgactcagaagaggaaatccgggaggcgttcagagtcttcgataaggatggcaatggttatataagcgcggcagaattgcggcatgtaatgacaaatcttggagagaaacttacagatgaggaggttgatgagatgatacgtgaagcagatatcgatggtgacggtcaagtgaattacgaagaatttgtccagatgatgacagcagggggtagctcaggcttacttacactggcgtctagacagcagttaatcgactggatggaagctgataaggtagccgggccattgttacggagcgcgttacctgcggggtggtttatcgcggataagagcggagcgggggaacggggttctagagggatcattgctgctttgggaccggacggaaagccgagtcgcatcgtggtaatatatacaactggttctcaggccacaatggatgagcggaatagacagatagccgaaattggcgctagcttaataaagcactggggcaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 73 - protein, 237 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKL

SEQ ID NO: 74 - protein, 384 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWASTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAASCRSKKTYPNDKTIISTFKWSYTTGNGKRYRSTARTTYTFAKPMAANYLKNQPMYVFRKTELKHSKTELNFKEWQKAFTDVMGMDELYKL

SEQ ID NO: 75 - protein, 530 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWASTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAASCRSKKTYPNDKTIISTFKWSYTTGNGKRYRSTARTTYTFAKPMAANYLKNQPMYVFRKTELKHSKTELNFKEWQKAASTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAASTDVMGMDELYKL

SEQ ID NO: 76 - protein, 195 aa, synthetic construct
SPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGS

SEQ ID NO: 77 - protein, 7 aa, synthetic construct
EDAPESG

SEQ ID NO: 78 - protein, 713 aa, synthetic construct
MDTSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLEVLFQGPTSLYKKAGLMDVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKL

SEQ ID NO: 79 - protein, 918 aa, synthetic construct
MDTSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLEVLFQGPTSLYKKAGLMDVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKL

SEQ ID NO: 80 - protein, 917 aa, synthetic construct
MDTSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLEVLFQGPTSLYKKAGLMDVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYANYSAAANKTIKDLAQNGVKVAAGVPVTKESEWTGKNFVPPLKTLYTVQDYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKL

SEQ ID NO: 81 - protein, 1122 aa, synthetic construct
MDTSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLEVLFQGPTSLYKKAGLMDVPLIPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYANYSAAANKTIKDLAQNGVKVAAGVPVTKESEWTGKNFVPPLKTLYTVQDEDAPESGSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRGSGPGYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKL

SEQ ID NO: 82 - protein, 8 aa, synthetic construct
RGSIDTWV

SEQ ID NO: 83 - protein, 461 aa, synthetic construct
MKKFVALLITMFFVVSMAAVAQPASAAKYSELEEGGVIMQAFYWDVPAGGIWWDTIRSKIPEWYEAGISAIWIPPASKGMGGAYSMGYDPYDFFDLGEYNQKGTVETRFGSKQELINMINTAHAYGIKVIADIVINHRAGGDLEWNPFVGDYTWTDFSKVASGKYTANYLDFHPNEVKCCDEGTFGGFPDIAHEKEWDQHWLWASDESYAAYLRSIGVDAWRFDYVKGYGAWVVKDWLNWWGGWAVGEYWDTNVDALLNWAYSSGAKVFDFPLYYKMDEAFDNTNIPALVDALQNGGTVVSRDPFKAVTFVANHDTDIIWNKYPAYAFILTYEGQPVIFYRDYEEWLNKDKLNNLIWIHDHLAGGSTSIVYYDSDELIFVRNGDSKRPGLITYINLGSSKVGRWVYVPKFAGACIHEYTGNLGGWVDKYVESSGWVYLEAPAYDPASGQYGYTVWSYCGVG

SEQ ID NO: 84 - protein, 1056 aa, synthetic construct
DGSQVQLVESGGGLVQAGGSLRLSCAASRRSSRSWAMAWFRQAPGKEREFVAKISGDGRLTTYGDSVKGRFTISRDNAEYLVYLQMDSLKPEDTAVYYCAADDNYVTASWRSGPDYWGQGTQVTVSSGGGSGGSGSGSGGSGGDVPLIPSQFAKAKSENFDKKVILSNLNKPHALLWGPDNQIWLTERATGCGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSCGGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDGCGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSCGNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKGGSGGSGSGSGGSGGGGSQVQLVESGGGLVQAGGSLRLSCAASRRSSRSWAMAWFRQAPGKEREFVAKISGDGRLTTYGDSVKGRFTISRDNAEYLVYLQMDSLKPEDTAVYYCAADDNYVTASWRSGPDYWGQGTQVTVSSKLAAALEHHHHHH

SEQ ID NO: 85 - protein, 278 aa, synthetic construct
DHPETLVKVKDAEDQLGARVGYIELDLNSGQILESFRPEERFPMMSTFKVLLCGAVLARVDAGQEQLDRRIHYSQNDLVEYSPVTEKHLTDGMTVRELCSAAITMSDNTAANLLLKTIGGPKELTAFLRNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGKLAAALEHHHHHH

SEQ ID NO: 86 - protein, 580 aa, synthetic construct
DHPETLVKVKDAEDQLGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGARVGYIELDLNSGQILESFRPEERFPMMSTFKVLLCGAVLARVDAGQEQLDRRIHYSQNDLVEYSPVTEKHLTDGMTVRELCSAAITMSDNTAANLLLKTIGGPKELTAFLRNMGDHVTRLDRWEPELNEAIPNDERDTTMPAAMATTLRKLLTGGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGLLTLASRQQLIDWMEADKVAGPLLRSALPAGWFIADKSGAGERGSRGIIAALGPDGKPSRIVVIYTTGSQATMDERNRQIAEIGASLIKHWGKLAAALEHHHHHH

SEQ ID NO: 87 - protein, 852 aa, synthetic construct
DGSQVQLVESGGGLVQAGGSLRLSCAASRRSSRSWAMAWFRQAPGKEREFVAKISGDGRLTTYGDSVKGRFTISRDNAEYLVYLQMDSLKPEDTAVYYCAADDNYVTASWRSGPDYWGQGTQVTVSSGGSGGGGSGGSGGHPETLVKVKDAEDQLGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGARVGYIELDLNSGQILESFRPEERFPMMSTFKVLLCGAVLARVDAGQEQLDRRIHYSQNDLVEYSPVTEKHLTDGMTVRELCSAAITMSDNTAANLLLKTIGGPKELTAFLRNMGDHVTRLDRWEPELNEAIPNDERDTTMPAAMATTLRKLLTGGSGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSSGLLTLASRQQLIDWMEADKVAGPLLRSALPAGWFIADKSGAGERGSRGIIAALGPDGKPSRIVVIYTTGSQATMDERNRQIAEIGASLIKHWGGSGGSGGGSQVQLVESGGGLVQAGGSLRLSCAASRRSSRSWAMAWFRQAPGKEREFVAKISGDGRLTTYGDSVKGRFTISRDNAEYLVYLQMDSLKPEDTAVYYCAADDNYVTASWRSGPDYWGQGTQVTVSSKLAAALEHHHHHH

SEQ ID NO: 88 - protein, 190 aa, synthetic construct
DGSSHHHHHHNPSLIRSESWIPYFGNEANLLDGDDNTGVWYLKDSYEFSLAGEFIGLDLGKEIKLDGIRFVIGKNGGGSSDKWNKFKLEYSLDNESWTTIKEYDKTGAPAGKDVIEESFETPISAKYIRLTNLEQIFHALTFSEFAIVSDGGSGSGGSGGGSGSSGGSGGSGGGKRRWKKNFIAVASASA

SEQ ID NO: 89 - protein, 771 aa, synthetic construct
DVPLIPSQFAKAKSENFDKKVILSNLNKPHALLWGPDNQIWLTERATGCGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSCGGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDGCGGTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAGGSCGNPSFNGVVSHIYTLGHRNPQGLAFTPNGKLLQSEQGPNSDDEINLIVKGGNYGWPNVAGYKDDSGYAYANYSAAANKTIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAKKLAAALEHHHHHH